(19) United States
(12) Patent Application Publication
(10) Pub. No.: US 2015/0045540 A1
(43) Pub. Date: Feb. 12, 2015

(54) MULTISPECIFIC STACKED VARIABLE DOMAIN BINDING PROTEINS

(71) Applicant: SEA LANE BIOTECHNOLOGIES, LLC, Mountain View, CA (US)

(72) Inventors: Lawrence C. Howowitz, Mountain View, CA (US); Ramesh R. Bhatt, Mountain View, CA (US); Li Xu, Cupertino, CA (US); Arun K. Kashyap, Newark, CA (US); Sandra M. Wang, Mountain View, CA (US); Pamela Foreman, Los Altos, CA (US); Medini Gore, Los Altos, CA (US); Phil Kobel, Los Gatos, CA (US); Danying Cai, Redwood City, CA (US)

(21) Appl. No.: 14/128,455

(22) PCT Filed: Jun. 28, 2012
(86) PCT No.: PCT/US2012/044746

§ 371 Date: Aug. 29, 2014

Related U.S. Application Data
(60) Provisional application No. 61/502,293, filed on Jun. 28, 2011.
(60) Provisional application No. 61/640,467, filed on Apr. 30, 2012.

(51) Int. Cl. C07K 16/46; C07K 16/28; C07K 16/22

(52) U.S. Cl. CPC C07K 16/468; C07K 16/22; C07K 16/2863; C07K 16/2809; C07K 2317/31; C07K 2317/62; C07K 2317/76; A61K 2039/505
USPC 530/387.3; 530/391.7

(57) ABSTRACT

The present invention concerns multi-specific stacked variable domain binding proteins.

### RELATED APPLICATIONS

This application claims the benefit of U.S. Provisional Application No. 61/502,293 filed Jun. 28, 2011 and 61/640,467 filed Apr. 30, 2012, the contents of which are incorporated herein by reference in their entirety.

### FIELD OF THE INVENTION

The present invention concerns multispecific stacked variable domain binding proteins and methods of making the same.

### BACKGROUND OF THE INVENTION

Significant efforts have been directed to the development of immunoglobulin-based therapeutics having more than one antigen binding specificity, e.g., bispecific antibodies. It is well known that multiple molecules may play a role in the pathogenesis of disease. The simultaneous blockade of such molecules may provide better clinical efficacy and reach a broader patient population than inhibition of a single target (Wu et al. 2007 Nature Biotech. 25(11):1291-1297). For example, a bispecific antibody might have one specificity to target a tumor cell antigen and another specificity to trigger a response by the immune system. A variety of recombinant antibody and antibody fragment formats have been developed in the art to address such therapeutic opportunities. Wu et al. U.S. Pat. No. 7,612,181 describe a dual variable domain immunoglobulin made up of four polypeptides. Two of the polypeptides have two heavy chain variable domains and the other two polypeptides have two light chain variable domains. Binding sites to more than one antigen are formed as shown in FIG. 1A of Wu et al. There remains a need for alternative formats for multispecific binding molecules.

SURROBODIES™ (Surroglobulins) are a new class of binding molecules which utilize surrogate light chain sequences. Surrobodies are based on the pre-B cell receptor (pre-BCR), which is produced during normal development of antibody repertoire. Precursors of B cells (pre-B cells) have been identified in the bone marrow by their production of a set of genes called VpreB(1-3) and λ5, instead of the fully developed light chains, and coexpression of μ heavy chains. The VpreB and λ5 polypeptides together form a non-covalently associated, Ig light chain-like structure, which is called the surrogate light chain or pseudo light chain. Both VpreB and λ5 are encoded by genes that do not undergo gene rearrangement and are expressed in early pre-B cells before V(D)J recombination begins. The pre-BCR is structurally different from a mature immunoglobulin in that it is composed of a heavy chain and two non-covalently associated proteins: VpreB and λ5, i.e., they have three components as opposed to two in antibodies.

A κ-like B cell receptor (κ-like BCR) has also been identified, utilizing a κ-like surrogate light chain (κ-like SLC) (Frances et al., EMBO J 13:5937-43 (1994); Thompson et al., Immunogenetics 48:305-11 (1998); Rangel et al., J Biol Chem 280:17807-14 (2005)). Rangel et al., supra report the identification and molecular characterization of a Vκ-like protein that is the product of an unrearranged Vκ gene, which turned out to the be identical to the cDNA sequence previously reported by Thompson et al., supra. Whereas, Frances et al., supra reported the identification and characterization of a rearranged germ line JCk that has the capacity to associate with u heavy chains at the surface of B cell precursors, thereby providing an alternative to the λ5 pathway for B cell development. It has been proposed that κ-like and λ-like pre-BCRs work in concert to promote light chain rearrangement and ensure the maturation of B cell progenitors. For a review, see McKeller and Martinez-Valdez Seminars in Immunology 18:4043 (2006).

Further details of the design and production of Surrobodies® (Surroglobulins) are provided in Xu et al., Proc. Natl. Acad. Sci. USA 2008, 105(31):10756-61, Xu et al., J Mol. Biol. 2010, 397, 352-360, and in PCT Publication Nos. WO 2008/118970 published on Oct. 2, 2008; WO/2010/006286 published on Jan. 14, 2010; and WO/2010/151808 published on Dec. 29, 2010, the disclosures of which are incorporated by reference herein in their entirety.

The present invention concerns surrogate light chain-based multi-specific stacked variable domain binding proteins, as well as and methods and means for making and using such binding proteins.

### SUMMARY OF THE INVENTION

The invention concerns multispecific stacked variable domain binding proteins (e.g., FIGS. 1A-F, 17-19, and 21). In one embodiment, the invention is directed to the following set of potential claims to multispecific SVD binding proteins with heavy chain variable domain-surrogate light chain tandem products (e.g., FIG. 1A) for this application:

1. A multi-specific Stacked Variable Domain (SVD) binding protein comprising a tandem product of a first heavy chain variable domain sequence conjugated to a second surrogate light chain sequence, associated with a first surrogate light chain sequence conjugated to a second heavy chain variable domain sequence, wherein the tandem product comprises a first binding domain and a second binding domain, wherein each of said first and second binding domains is formed by a surrogate light chain sequence and an antibody variable domain sequence, and wherein each of said first and second binding domains binds specifically to a different binding target.
2. The multi-specific SVD binding protein of claim 1, wherein said first and said second binding domains are present in a single polypeptide chain.
3. The multi-specific SVD binding protein of claim 1, wherein said first and said second binding domains are present on more than one polypeptide chain.
4. The multi-specific SVD binding protein of claim 1, wherein the C-terminus of said first heavy chain variable domain sequence is conjugated to the N-terminus of said second surrogate light chain sequence.
5. The multi-specific SVD binding protein of claim 1, wherein the C-terminus of said first surrogate light chain sequence is conjugated to the N-terminus of said second heavy chain variable domain sequence.
6. The multi-specific SVD binding protein of claim 1, wherein said first heavy chain variable domain sequence and said first surrogate light chain sequence together form a first binding domain specifically binding to a first target.
7. The multi-specific SVD binding protein of claim 1 or claim 6, wherein said second surrogate light chain sequence and said second heavy chain variable domain sequence together form a second binding domain specifically binding to a second target.
8. The multi-specific SVD binding protein of any one of claims 1 to 7, wherein said first and said second surrogate light chain sequences are identical.
9. The multi-specific SVD binding protein of any one of claims 1 to 7, wherein said first and said second surrogate light chain sequences are different.
10. The multi-specific SVD binding protein of claim 8 or claim 9 wherein said first and said second surrogate light chain sequences comprise a VpreB sequence.
11. The multi-specific SVD binding protein of claim 10 wherein said second surrogate light chain sequence further comprises a λ5 sequence.
12. The multi-specific SVD binding protein of any one of claims 1 to 11, wherein said second heavy chain variable domain sequence further comprises a heavy chain constant domain sequence.
13. The multi-specific SVD binding protein of claim 12, wherein said second heavy chain variable domain sequence further comprises a CH1 sequence.
14. The multi-specific SVD binding protein of claim 12, wherein said second heavy chain variable domain sequence further comprises an Fc region.
15. The multi-specific SVD binding protein of claim 1, wherein the association is covalent and/or non-covalent.
16. The multi-specific SVD binding protein of any one of claims 1 to 15, wherein the conjugation is by a linker sequence.
17. The multi-specific SVD binding protein of claim 16, wherein the linker sequence is heterologous linker sequence.
18. The multi-specific SVD binding protein of any one of claims 1 to 15, wherein the conjugation is direct fusion.
19. The multi-specific SVD binding protein of claim 16, wherein the linker sequence comprises a sequence selected from the group consisting of: an antibody J region sequence, a λ5 sequence, a λ light chain constant region sequence, a κ light chain constant region sequence, synthetic sequence, and any combination thereof.
20. The multi-specific SVD binding protein of claim 19, wherein the synthetic sequence is (Gly-Gly-Gly-Ser)ₙ (SEQ ID NO: 109), (Gly-Gly-Gly-Gly-Ser)ₙ (SEQ ID NO: 110), or Gly-Ala, wherein n is at least 1.
21. The multi-specific SVD binding protein of claim 18, wherein the C-terminus of the heavy chain variable domain sequence of a first Surrobody is fused to the N-terminus of the surrogate light chain sequence of a second Surrobody forming a first polypeptide chain.
22. The multi-specific SVD binding protein of claim 18, wherein the C-terminus of the surrogate light chain sequence of the first surrobody is fused to the N-terminus of the heavy chain variable domain sequence of a second surrobody forming a second polypeptide chain.
23. The multi-specific SVD binding protein of claim 21 or 22, wherein a binding site to a target is formed between a surrogate light chain sequence and a heavy chain variable domain sequence on different polypeptide chains.
24. The multi-specific SVD binding protein of claim 21 or 22, wherein a binding site to a target is formed between a surrogate light chain sequence and a heavy chain variable domain sequence on the same polypeptide chains.
25. The multi-specific SVD binding protein of claim 18, wherein the C-terminus of the first heavy chain variable domain sequence is fused to the N-terminus of the second surrogate light chain sequence forming a first polypeptide chain.
26. The multi-specific SVD binding protein of claim 18, wherein the C-terminus of the second surrogate light chain sequence is fused to the N-terminus of the second heavy chain variable domain sequence forming a second polypeptide chain.
27. The multi-specific SVD binding protein of claim 25 or 26, wherein a binding site to a target is formed between a surrogate light chain sequence and a heavy chain variable domain sequence on different polypeptide chains.
28. The multi-specific SVD binding protein of claim 25 or 26, wherein a binding site to a target is formed between a surrogate light chain sequence and a heavy chain variable domain sequence on the same polypeptide chain.
29. A multi-specific SVD binding protein as substantially described herein with reference to and as illustrated by any of the accompanying drawings.

In another embodiment, the invention is directed to the following set of potential claims for heteromeric multispecific binding proteins comprising polypeptide chains (e.g., FIG. 1A) in this application:

1. A first polypeptide chain comprising an antibody heavy chain variable region sequence, specific for a first target, C-terminally conjugated to a polypeptide sequence comprising a VpreB sequence.
2. The polypeptide chain of claim 1 associated with a second polypeptide chain comprising a VpreB sequence, conjugated to the N-terminus of an antibody heavy chain comprising a variable region sequence specific for a second target.
3. The polypeptide chain of claim 2, wherein the antibody heavy chain variable region sequence of the first polypeptide chain and the VpreB sequence of the second polypeptide chain form a binding site for said first target.
4. A heteromeric bispecific binding protein comprising the first polypeptide chain of claim 1, associated with the second polypeptide of claim 2.
5. The heteromeric bispecific binding protein of claim 4, wherein the heavy chain variable region of the second antibody heavy chain variable region sequence specific for said second target and the VpreB sequence of the first polypeptide chain form a binding site for a second target.
6. A heteromeric bispecific binding protein comprising two pairs of the polypeptide of claim 2, associated with each other, or one pair of the first polypeptide chain of claim 1 and one pair of the second polypeptide chain of claim 2.
7. The heteromeric bispecific binding protein of claim 6, wherein the heavy chain variable region of the second antibody heavy chain variable region sequence specific for said second target and the VpreB sequence of the first polypeptide chain form a binding site for a second target.
8. The polypeptide chain of claim 1 or 2 or the heteromeric bispecific binding protein of claim 4 or 5, wherein in the first polypeptide chain the conjugation is by a linker sequence.
9. The polypeptide chain or heteromeric bispecific binding protein of claim 8, wherein the linker sequence is a heterologous linker sequence.
10. The polypeptide chain of claim 1 or 2 or the heteromeric bispecific binding protein of claim 4 or 5, wherein in the first polypeptide chain the conjugation is direct fusion.
11 The polypeptide chain of claim 1 or 2 or the heteromeric bispecific binding protein of claim 4 or 5, wherein in the second polypeptide chain the conjugation is by a linker sequence.
12. The polypeptide chain or heteromeric bispecific binding protein of claim 11, wherein the linker sequence is a heterologous linker sequence.
13. The polypeptide chain of claim 1 or 2 or the heteromeric bispecific binding protein of claim 4 or 5, wherein in the second polypeptide chain the conjugation is direct fusion.
14. The polypeptide chain of claim 8 or 11, wherein the linker sequence between the antibody heavy chain variable region sequence and the VpreB sequence of the first polypeptide chain comprises a sequence selected from the group consisting of: an antibody J region sequence, an antibody constant domain region sequence, a synthetic sequence, and any combination thereof.
15. The polypeptide chain of claim 8 or 11, wherein the linker sequence between the antibody heavy chain variable region sequence and the VpreB sequence of the first polypeptide chain comprises a sequence selected from the group consisting of:

(SEQ ID NO: 67)

Xaa_{g} Ala Ser Xaaₕ,

(SEQ ID NO: 68)

Xaa_{g} Ala Ser Thr Xaaₕ,

(SEQ ID NO: 69)

Xaa_{g} Ala Ser Thr Lys Xaaₕ,

(SEQ ID NO: 70)

Xaa_{g} Ala Ser Thr Lys Gly Xaaₕ,

(SEQ ID NO: 71)

Xaa_{g} Ala Ser Thr Lys Gly Pro Xaaₕ,

(SEQ ID NO: 72)

Xaa_{g} Ala Ser Thr Lys Gly Pro Ser Xaaₕ,

(SEQ ID NO: 73)

Xaa_{g} Ala Ser Thr Lys Gly Pro Ser Val Xaaₕ,

(SEQ ID NO: 74)

Xaa_{g} Ala Ser Thr Lys Gly Pro Ser Val Phe Xaaₕ,
and

(SEQ ID NO: 75)

Xaa_{g} Ala Ser Thr Lys Gly Pro Ser Val Phe Pro Xaaₕ,

wherein the Xaa is any amino acid, g is 0 to 10 amino acids, and h is 0 to 10 amino acids.
16. The polypeptide chain of claim 15, wherein Xaa_{g} comprises a sequence selected from the group consisting of

Ser,
Ser Ser,
Val Ser Ser,

(SEQ ID NO: 76)

Thr Val Ser Ser,

(SEQ ID NO: 77)

Val Thr Val Ser Ser,

(SEQ ID NO: 78)

Leu Val Thr Val Ser Ser,

(SEQ ID NO: 79)

Thr Leu Val Thr Val Ser Ser,

(SEQ ID NO: 80)

Gly Thr Leu Val Thr Val Ser Ser,

(SEQ ID NO: 81)

Gln Gly Thr Leu Val Thr Val Ser Ser,
and

(SEQ ID NO: 82)

Gly Gln Gly Thr Leu Val Thr Val Ser Ser.

17. The polypeptide chain of claim 15, wherein Xaaₕ comprises a sequence selected from the group consisting of

Gln,
Gln Pro,
Gln Pro Val,

(SEQ ID NO: 128)

Gln Pro Val Leu,

(SEQ ID NO: 83)

Gln Pro Val Leu His,

(SEQ ID NO: 84)

Gln Pro Val Leu His Gln,

(SEQ ID NO: 85)

Gln Pro Val Leu His Gln Pro,

(SEQ ID NO: 86)

Gln Pro Val Leu His Gln Pro Pro,

(SEQ ID NO: 87)

Gln Pro Val Leu His Gln Pro Pro Ala,
and

(SEQ ID NO: 88)

Gln Pro Val Leu His Gln Pro Pro Ala Met.

18. The polypeptide chain of claim 8 or 11, wherein the linker sequence between the antibody heavy chain variable region sequence and the VpreB sequence of the second polypeptide chain comprises a sequence selected from the group consisting of: a λ5 sequence, an antibody J region sequence, a λ light chain constant region sequence, a κ light chain constant region sequence, a synthetic sequence, and any combination thereof.
19. The polypeptide chain of claim 8 or 11, wherein the linker sequence between the antibody heavy chain variable region sequence and the VpreB sequence of the second polypeptide chain comprises a sequence selected from the group consisting of:

(SEQ ID NO: 89)

Xaaⱼ Ser Gln Xaaₖ,

(SEQ ID NO: 90)

Xaaⱼ Ser Gln Pro Xaaₖ,

(SEQ ID NO: 91)

Xaaⱼ Ser Gln Pro Lys Xaaₖ,

(SEQ ID NO: 92)

Xaaⱼ Ser Gln Pro Lys Ala Xaaₖ,

(SEQ ID NO: 93)

Xaaⱼ Ser Gln Pro Lys Ala Thr Xaaₖ,

(SEQ ID NO: 94)

Xaaⱼ Ser Gln Pro Lys Ala Thr Pro Xaaₖ,

(SEQ ID NO: 95)

Xaaⱼ Ser Gln Pro Lys Ala Thr Pro Ser Xaaₖ,

(SEQ ID NO: 96)

Xaaⱼ Ser Gln Pro Lys Ala Thr Pro Ser Val Xaaₖ,

(SEQ ID NO: 97)

Xaaⱼ Ser Gln Pro Lys Ala Thr Pro Ser Val Thr Xaaₖ,
and

(SEQ ID NO: 98)

Xaaⱼ Ser Gln Pro Lys Ala Thr Pro Ser Val Thr Gly
Gly Gly Gly Ser Xaaₖ,

wherein Xaa is any amino acid, j is 0 to 10 amino acids, and k is 0 to 6 amino acids.
20. The polypeptide chain of claim 19, wherein Xaaⱼ comprises a sequence selected from the group consisting of

Leu,
Val Leu,
Thr Val Leu,

(SEQ ID NO: 99)

Leu Thr Val Leu,

(SEQ ID NO: 100)

Gln Leu Thr Val Leu,

(SEQ ID NO: 101)

Thr Gln Leu Thr Val Leu,

(SEQ ID NO: 102)

Gly Thr Gln Leu Thr Val Leu,

(SEQ ID NO: 103)

Ser Gly Thr Gln Leu Thr Val Leu,
and

(SEQ ID NO: 104)

Gly Ser Gly Thr Gln Leu Thr Val Leu.

21. The polypeptide chain of claim 19, wherein Xaaₖ comprises a sequence selected from the group consisting of

Gln,
Gln Val,
Gln Val Gln,

(SEQ ID NO: 105)

Gln Val Gln Leu,

(SEQ ID NO: 106)

Gln Val Gln Leu Val,
and

(SEQ ID NO: 107)

Gln Val Gln Leu Val Gln.

22. The polypeptide chain of claim 2 or the heteromeric bispecific binding protein of claim 6, wherein the association is covalent or non-covalent.
23. The polypeptide chain of claim 1 or 2, or the heteromeric bispecific binding protein of claim 4 or 5, wherein the VpreB sequence is fused, at its C-terminus, to a heterologous sequence.
24. The polypeptide chain or the heteromeric bispecific binding protein of claim 23, wherein the heterogenous sequence is selected from the group consisting of a λ5 sequence, an antibody J-region sequence, and a light chain constant domain region sequence.

In one other embodiment, the invention is directed to the following set of potential claims for heteromeric bispecific binding proteins comprising polypeptide chains (e.g., FIG. 1B) in this application:

1. A first polypeptide chain comprising an antibody heavy chain variable region sequence specific for a first target, C-terminally conjugated to a first polypeptide sequence comprising a first VpreB sequence, wherein the first polypeptide sequence comprising the VpreB sequence is C-terminally conjugated to a second polypeptide sequence comprising a second VpreB sequence, conjugated to a heterologous sequence.
2. The polypeptide chain of claim 1 associated with a second polypeptide chain comprising an antibody heavy chain comprising a variable region sequence specific for a second polypeptide target.
3. The polypeptide chain of claim 1, wherein the antibody heavy chain variable region sequence of the first polypeptide chain and the first VpreB sequence of the first polypeptide chain form a binding site for said first target.
4. A heteromeric bispecific binding protein comprising two pairs of the polypeptide of claim 2, associated with each other, or one pair of the first polypeptide chain of claim 1 and one pair of the second polypeptide chain of claim 2.
5. The heteromeric bispecific binding protein of claim 4, wherein the heavy chain variable region of the second antibody heavy chain variable region sequence specific for said second target and the second VpreB sequence of the first polypeptide chain form a binding site for a second target.
6. The polypeptide chain of claim 1 or 2 or the heteromeric bispecific binding protein of claim 3 or 4, wherein in the first polypeptide chain the conjugation is by a linker sequence.
7. The polypeptide chain or heteromeric bispecific binding protein of claim 6, wherein the linker sequence is heterologous linker sequence.
8. The polypeptide chain of claim 1 or 2 or the heteromeric bispecific binding protein of claim 3 or 4, wherein in the first polypeptide chain the conjugation is direct fusion.
9. The polypeptide chain of claim 6, wherein the linker sequence between the antibody heavy chain variable region sequence and the first polypeptide sequence comprising a first VpreB sequence comprises the amino acid sequence Gly-Gly-Gly-Gly-Ser (SEQ ID NO: 108).
10. The polypeptide chain of claim 6, wherein the linker sequence between the first polypeptide sequence comprising a first VpreB sequence and the second polypeptide sequence comprising a second VpreB sequence comprises the amino acid sequence Gly-Ala.
11. The polypeptide chain of claim 2 or the heteromeric bispecific binding protein of claim 4, wherein the association is covalent or non-covalent.
12. The polypeptide chain of claim 1 or 2, or the heteromeric bispecific binding protein of claim 4 or 5, wherein the VpreB sequence is fused, at its C-terminus, to a heterologous sequence.
13. The polypeptide chain or the heteromeric bispecific binding protein of claim 12, wherein the heterogenous sequence is selected from the group consisting of a λ5 sequence and a light chain constant domain region sequence.

In yet another embodiment, the invention is directed to the following set of potential claims for multispecific SVD binding proteins having an scSv component and an SLC, which comprise polypeptide chains (e.g., FIG. 1C) this application:

1. A first polypeptide chain comprising a first polypeptide sequence comprising a second VpreB sequence, C-terminally conjugated to a second polypeptide sequence comprising a first VpreB sequence, wherein the second polypeptide sequence is C-terminally conjugated to a first antibody heavy chain variable region sequence specific for a first target.
2. The polypeptide chain of claim 1 associated with a second polypeptide chain comprising a second antibody heavy chain variable region sequence specific for a second target.
3. The polypeptide chain of claim 1, wherein the first antibody heavy chain variable region sequence and the second VpreB sequence form a binding site for said first target.
4. A heteromeric bispecific binding protein comprising the first polypeptide chain of claim 1, associated with the second polypeptide of claim 2.
5. The heteromeric bispecific binding protein of claim 4, wherein the second antibody heavy chain variable region sequence of the second polypeptide chain and the first VpreB sequence of the first polypeptide chain form a binding site for said second target.
6. The polypeptide chain of claim 1 or 2 or the heteromeric bispecific binding protein of claim 4 or 5, wherein in the first polypeptide chain the conjugation is by a linker sequence.
7. The polypeptide chain or heteromeric bispecific binding protein of claim 6, wherein the linker sequence is heterologous linker sequence.
8. The polypeptide chain of claim 1 or 2 or the heteromeric bispecific binding protein of claim 4 or 5, wherein in the first polypeptide chain the conjugation is direct fusion.
9 The polypeptide chain of claim 1 or 2 or the heteromeric bispecific binding protein of claim 4 or 5, wherein in the second polypeptide chain the conjugation is by a linker sequence.
10. The polypeptide chain or heteromeric bispecific binding protein of claim 9, wherein the linker sequence is a heterologous linker sequence.
11. The polypeptide chain of claim 6 or 9, wherein the linker sequence between the antibody heavy chain variable region sequence and the VpreB sequence of the first polypeptide chain comprises a sequence selected from the group consisting of: an antibody J region sequence, an antibody constant domain region sequence, a synthetic sequence, and any combination thereof.

In yet another embodiment, the invention is directed to the following set of potential claims for multispecific SVD binding proteins having an scSv component and an SLC, which comprise polypeptide chains (e.g., FIG. 1D) this application:

1. A first polypeptide chain comprising a first antibody heavy chain variable region sequence specific for a first target, C-terminally conjugated to a first polypeptide sequence comprising a first VpreB sequence, wherein the first VpreB sequence is C-terminally conjugated to a second antibody heavy chain variable region sequence specific for a second target.
2. The polypeptide chain of claim 1 associated with a second polypeptide chain comprising a second VpreB sequence.
3. The polypeptide chain of claim 1, wherein the first antibody heavy chain variable region sequence and the first VpreB sequence form a binding site for said first target.
4. A heteromeric bispecific binding protein comprising the first polypeptide chain of claim 1, associated with the second polypeptide of claim 2.
5. The heteromeric bispecific binding protein of claim 4, wherein the second antibody heavy chain variable region sequence of the first polypeptide chain and the second VpreB sequence of the second polypeptide chain final a binding site for said second target.
6. The polypeptide chain of claim 1 or 2 or the heteromeric bispecific binding protein of claim 4 or 5, wherein in the first polypeptide chain the conjugation is by a linker sequence.
7. The polypeptide chain or heteromeric bispecific binding protein of claim 6, wherein the linker sequence is heterologous linker sequence.
8. The polypeptide chain of claim 1 or 2 or the heteromeric bispecific binding protein of claim 4 or 5, wherein in the first polypeptide chain the conjugation is direct fusion.
9 The polypeptide chain of claim 1 or 2 or the heteromeric bispecific binding protein of claim 4 or 5, wherein in the second polypeptide chain the conjugation is by a linker sequence.
10. The polypeptide chain or heteromeric bispecific binding protein of claim 9, wherein the linker sequence is a heterologous linker sequence.
11. The polypeptide chain of claim 6 or 9, wherein the linker sequence between the antibody heavy chain variable region sequence and the VpreB sequence of the first polypeptide chain comprises a sequence selected from the group consisting of: an antibody J region sequence, an antibody constant domain region sequence, a synthetic sequence, and any combination thereof.

In yet another embodiment, the invention is directed to the following set of potential claims for multispecific SVD binding proteins having an scSv component and an SLC, which comprise polypeptide chains (e.g., FIG. 1E) this application:

1. A first polypeptide chain comprising a first antibody heavy chain variable domain sequence specific for a first target, C-terminally conjugated to a first polypeptide sequence comprising a first VpreB sequence, wherein the first VpreB sequence is C-terminally conjugated to a second antibody heavy chain variable region sequence specific for a second target, wherein the first antibody heavy chain variable domain sequence further comprises an antibody heavy chain constant domain sequence.
2. The polypeptide chain of claim 1, wherein the N-terminus of the antibody heavy chain constant domain sequence is conjugated to the C-terminus of the first antibody heavy chain variable domain sequence and the C-terminus of the antibody HC constant domain sequence is conjugated to the N-terminus of the first polypeptide sequence comprising the first VpreB sequence.
3. The polypeptide chain of claim 1 or 2, wherein the antibody heavy chain constant domain sequence comprises a CH1 sequence and/or an Fc region.
4. The polypeptide chain of claim 1 associated with a second polypeptide chain comprising a second VpreB sequence.
5. The polypeptide chain of claim 1, wherein the first antibody heavy chain variable region sequence and the first VpreB sequence form a binding site for said first target.
6. A heteromeric bispecific binding protein comprising the first polypeptide chain of claim 1, associated with the second polypeptide of claim 4.
7. The heteromeric bispecific binding protein of claim 6, wherein the second antibody heavy chain variable region sequence of the first polypeptide chain and the second VpreB sequence of the second polypeptide chain form a binding site for said second target.
8. The polypeptide chain of claim 1 or 4 or the heteromeric bispecific binding protein of claim 6 or 7, wherein in the first polypeptide chain the conjugation is by a linker sequence.
9. The polypeptide chain or heteromeric bispecific binding protein of claim 8, wherein the linker sequence is heterologous linker sequence.
10. The polypeptide chain of claim 1 or 4 or the heteromeric bispecific binding protein of claim 6 or 7, wherein in the first polypeptide chain the conjugation is direct fusion.
11 The polypeptide chain of claim 1 or 4 or the heteromeric bispecific binding protein of claim 6 or 7, wherein in the second polypeptide chain the conjugation is by a linker sequence.
12. The polypeptide chain or heteromeric bispecific binding protein of claim 11, wherein the linker sequence is a heterologous linker sequence.
13. The polypeptide chain of claim 8 or 11, wherein the linker sequence between the antibody heavy chain variable region sequence and the VpreB sequence of the first polypeptide chain comprises a sequence selected from the group consisting of: an antibody J region sequence, an antibody constant domain region sequence, a synthetic sequence, and any combination thereof.

In yet another embodiment, the invention is directed to the following set of potential claims for multispecific SVD binding proteins which comprise polypeptide chains (e.g., FIG. 17) in this application:

1. A polypeptide chain comprising an antibody heavy chain variable region sequence specific for a first target, C-terminally conjugated to a first polypeptide sequence comprising a first surrogate light chain (SLC) sequence, wherein the first SLC sequence is C-terminally conjugated to an antibody heavy chain variable region sequence specific for a second target.
2. The polypeptide chain of claim 1, wherein the antibody heavy chain variable region sequence specific for a second target is C-terminally conjugated to a second surrogate light chain (SLC) sequence.
3. The polypeptide chain of claim 2, wherein the second SLC sequence is conjugated to a dimerization domain.
4. The polypeptide chain of claim 3, wherein the dimerization domain comprises an antibody constant domain.
5. The polypeptide chain of claim 3, wherein the dimerization domain comprises an Fc region.
6. A multimeric bispecific binding protein of comprising one pair of the polypeptide of claim 3.
7. The polypeptide chain of claim 2 or the multimeric bispecific binding protein of claim 6, wherein the heavy chain variable region specific for the first target and the first SLC sequence form a binding site for the first target.
8. The multimeric bispecific binding protein of claim 7, wherein the heavy chain variable region specific for the second target and the second SLC sequence form a binding site for the second target.
9. The polypeptide chain or multimeric bispecific binding protein of any one of claims 1 to 8, wherein the conjugation is by a linker sequence.
10. The polypeptide chain or multimeric bispecific binding protein of claim 9, wherein the linker sequence is heterologous linker sequence.
11. The polypeptide chain or multimeric bispecific binding protein of any one of claims 1 to 8, wherein the conjugation is direct fusion.
12. The polypeptide chain or multimeric bispecific binding protein of claim 9, wherein the linker sequence comprises a sequence selected from the group consisting of: an antibody J region sequence, a λ5 sequence, a λ light chain constant region sequence, a κ light chain constant region sequence, synthetic sequence, and any combination thereof.
13. The polypeptide chain or multimeric bispecific binding protein of any one of claims 1 to 12, where the SLC sequence comprises a VpreB sequence.

In one embodiment, the invention is directed to the following set of potential claims for multispecific monomeric SVD binding protein (e.g., FIG. 18) in this application:

1. A first polypeptide chain comprising an antibody heavy chain variable region sequence specific for a first target conjugated to a first polypeptide sequence comprising a first VpreB sequence, wherein the first polypeptide sequence comprising the first VpreB sequence is C-terminally conjugated to a second polypeptide sequence comprising a dimerization domain.
2. The polypeptide chain of claim 1 associated with a second polypeptide chain comprising a first polypeptide sequence that comprises a second VpreB sequence, wherein the first polypeptide sequence comprising the second VpreB sequence is C-terminally conjugated to an antibody heavy chain variable region sequence specific for a second target.
3. The polypeptide chain of claim 2, wherein the antibody heavy chain variable region sequence specific for a second target comprises a dimerization domain.
4. The polypeptide chain of claim 1 or 3, wherein the dimerization domain comprises an antibody constant domain.
5. The polypeptide chain of claim 1 or 3, wherein the dimerization domain comprises an Fc region.
6. The polypeptide chain of claim 2, wherein the antibody heavy chain variable region sequence of the first polypeptide chain and the second VpreB sequence of the second polypeptide chain form a binding site for said first polypeptide target.
7. A heteromeric bispecific binding protein comprising the first and second polypeptide chains of any one of claims 2 to 6, associated with each other.
8. The heteromeric bispecific binding protein of claim 7, wherein the heavy chain variable region sequence specific for said second target of the second polypeptide and the first VpreB sequence of the first polypeptide chain form a binding site for a second target.
9. The polypeptide chain or heteromeric bispecific binding protein of any one of claims 1 to 8, wherein the conjugation is by a linker sequence.
10. The polypeptide chain or heteromeric bispecific binding protein of claim 9, wherein the linker sequence is heterologous linker sequence.
11. The polypeptide chain or heteromeric bispecific binding protein of any one of claims 1 to 8, wherein the conjugation is direct fusion.
12. The polypeptide chain or heteromultimeric bispecific binding protein of claim 9, wherein the linker sequence comprises a sequence selected from the group consisting of: an antibody J region sequence, a λ5 sequence, a light chain constant region sequence, a κ light chain constant region sequence, synthetic sequence, and any combination thereof
13. The polypeptide chain of claim 4 or 5, wherein the dimerization domain further comprises a protuberance or cavity.
14. The polypeptide chain of claim 1 or 2, or the heteromeric bispecific binding protein of claim 7 or 8, wherein the VpreB sequence is fused, at its C-terminus, to a heterologous sequence.
15. The polypeptide chain or the heteromeric bispecific binding protein of claim 14, wherein the heterogenous sequence is selected from the group consisting of a λ5 sequence and a light chain constant domain region sequence.
16. The polypeptide chain of claim 1 or 3, wherein one or both of the dimerization domains comprise an engineered amino acid sequence that promotes interaction between the dimerzation domains.
17. The polypeptide chain of claim 16, wherein the engineered amino acid sequence comprises a region selected from the group consisting of: a complementary hydrophobic region, a complementary hydrophilic region, and a compatible protein-protein interaction domain.

In another embodiment, the invention is directed to the following set of potential claims for multispecific monomeric SVD binding protein (e.g., FIG. 18) in this application:

1. A first polypeptide chain comprising an antibody heavy chain variable region sequence specific for a first target C terminally conjugated to a first polypeptide sequence comprising a first VpreB sequence, wherein the N-terminus of the antibody heavy chain variable region sequence specific for a first target is conjugated to a dimerization domain.
2. The polypeptide chain of claim 1 associated with a second polypeptide chain comprising a first polypeptide sequence that comprises a second VpreB sequence, wherein the C-terminus of the first polypeptide sequence comprising the second VpreB sequence is conjugated to an antibody heavy chain variable region sequence specific for a second target and the N-terminus of the first polypeptide sequence comprising the second VpreB sequence is conjugated to a dimerization domain.
3. The polypeptide chain of claim 1 or 2, wherein the dimerization domain comprises an antibody constant domain.
4. The polypeptide chain of claim 1 or 2, wherein the dimerization domain comprises an Fc region.
5. The polypeptide chain of claim 2, wherein the antibody heavy chain variable region sequence of the first polypeptide chain and the second VpreB sequence of the second polypeptide chain form a binding site for said first target.
6. A heteromeric bispecific binding protein comprising the first and second polypeptide chains of any one of claims 2 to 5, associated with each other.
7. The heteromeric bispecific binding protein of claim 6, wherein the heavy chain variable region sequence specific for said second target of the second polypeptide and the first VpreB sequence of the first polypeptide chain form a binding site for a second target.
8. The polypeptide chain or heteromeric bispecific binding protein of any one of claims 1 to 7, wherein the conjugation is by a linker sequence.
9. The polypeptide chain or heteromeric bispecific binding protein of claim 8, wherein the linker sequence is heterologous linker sequence.
10. The polypeptide chain or heteromeric bispecific binding protein of any one of claims 1 to 9, wherein the conjugation is direct fusion.
11. The polypeptide chain or heteromultimeric bispecific binding protein of claim 8, wherein the linker sequence comprises a sequence selected from the group consisting of: an antibody J region sequence, a λ5 sequence, a λ light chain constant region sequence, a κ light chain constant region sequence, synthetic sequence, and any combination thereof.
12. The polypeptide chain of claim 3 or 4, wherein the dimerization domain further comprises a protuberance or cavity.
13. The polypeptide chain of claim 1 or 2, or the heteromeric bispecific binding protein of claim 6 or 7, wherein the VpreB sequence is fused, at its C-terminus, to a heterologous sequence.
14. The polypeptide chain or the heteromeric bispecific binding protein of claim 13, wherein the heterogenous sequence is selected from the group consisting of a λ5 sequence, an antibody J-region sequence, and a light chain constant domain region sequence.
15. The polypeptide chain of claim 1 or 2, wherein one or both of the dimerization domains comprise an engineered amino acid sequence that promotes interaction between the dimerzation domains.
16. The polypeptide chain of claim 15, wherein the engineered amino acid sequence comprises a region selected from the group consisting of: a complementary hydrophobic region, a complementary hydrophilic region, and a compatible protein-protein interaction domain.

In one other embodiment, the invention is directed to the following set of potential claims for trispecific monomeric SVD binding proteins (e.g., FIG. 19) in this application:

1. A heteromeric trispecific binding protein comprising a first polypeptide chain comprising an antibody heavy chain variable region sequence specific for a first target, C-terminally conjugated to a polypeptide sequence comprising a first VpreB sequence, wherein the first polypeptide chain is associated with
a) a second polypeptide chain comprising a polypeptide sequence that comprises a second VpreB sequence conjugated to the N-terminus of an antibody heavy chain comprising a variable region sequence specific for a second target; and
b) a third polypeptide chain comprising a polypeptide sequence that comprises a third VpreB sequence conjugated to the N-terminus of an antibody heavy chain comprising a variable region sequence specific for a third target.
2. The heteromeric trispecific binding protein of claim 1, wherein the antibody heavy chain variable region sequence specific for a second target comprises a dimerization domain.
3. The heteromeric trispecific binding protein of claim 1, wherein the antibody heavy chain variable region sequence specific for a third target comprises a dimerization domain.
4. The heteromeric trispecific binding protein of claim 2 or 3, wherein the dimerization domain comprises an antibody constant domain.
5. The heteromeric trispecific binding protein of claim 2 or 3, wherein the dimerization domain comprises an Fc region.
6. The heteromeric trispecific binding protein of any one of claims 1 to 5, wherein the antibody heavy chain variable region sequence specific for a first target and the VpreB sequence of the second polypeptide chain form a binding site for said first target.
7. The heteromeric trispecific binding protein of any one of claims 1 to 6, wherein the antibody heavy chain variable region sequence specific for a first target and the VpreB sequence of the third polypeptide chain form a binding site for said first target.
8. The heteromeric trispecific binding protein of any one of claims 1 to 7, wherein the antibody heavy chain variable region sequence specific for a second target and the VpreB sequence of the first polypeptide chain form a binding site for said second target.
9. The heteromeric trispecific binding protein of any one of claims 1 to 8, wherein the antibody heavy chain variable region sequence specific for a third target and the VpreB sequence of the first polypeptide chain form a binding site for said third target.
10. The heteromeric trispecific binding protein of any one of claims 1 to 9, wherein the association is covalent or non-covalent.
11. The heteromeric trispecific binding protein of any one of claims 1 to 10, wherein the conjugation is by a linker sequence.
12. The heteromeric trispecific binding protein of claim 11, wherein the linker sequence is heterologous linker sequence.
13. The heteromeric trispecific binding protein of any one of claims 1 to 10, wherein the conjugation is direct fusion.
14. The heteromeric trispecific binding protein of claim 11, wherein the linker sequence comprises a sequence selected from the group consisting of: an antibody J region sequence, a XS sequence, a light chain constant region sequence, a κ light chain constant region sequence, synthetic sequence, and any combination thereof.
15. The heteromeric trispecific binding protein of claim 4 or 5, wherein the dimerization domain further comprises a protuberance or cavity.
16. The heteromeric trispecific binding protein of any one of claims 1 to 15, wherein the first VpreB sequence is fused, at its C-terminus, to a heterologous sequence.
17. The heteromeric trispecific binding protein of claim 16, wherein the heterogenous sequence is selected from the group consisting of a λ5 sequence and a light chain constant domain region sequence.
18. The polypeptide chain of claim 2 or 3, wherein one or both of the dimerization domains comprise an engineered amino acid sequence that promotes interaction between the dimerzation domains.
19. The polypeptide chain of claim 18, wherein the engineered amino acid sequence comprises a region selected from the group consisting of: a complementary hydrophobic region, a complementary hydrophilic region, and a compatible protein-protein interaction domain.

In yet another embodiment, the invention is directed to the following set of potential claims related to multispecific SVD molecules having a cross-complement format (e.g., FIG. 21) for this application:

1. A multi-specific Stacked Variable Domain (SVD) binding protein comprising a tandem product of a first heavy chain variable domain sequence conjugated to a second surrogate light chain sequence, associated with a second heavy chain variable domain sequence conjugated to a first surrogate light chain sequence, wherein the tandem product comprises a first binding domain and a second binding domain, wherein each of said first and second binding domains is fog Hied by a surrogate light chain sequence and an antibody variable domain sequence, wherein each of said first and second binding domains is formed between a surrogate light chain sequence and a heavy chain domain sequence on different polypeptide chains.
2. The multi-specific SVD binding protein of claim 1, wherein the first surrogate light chain sequence is further conjugated to an antibody heavy chain constant domain sequence.
3. The multi-specific SVD binding protein of claim 2, wherein said heavy chain variable domain sequence comprises a CH1 sequence and/or an Fc region.
4. The multi-specific SVD binding protein of claim 1, wherein the C-terminus of said first heavy chain variable domain sequence is conjugated to the N-terminus of said second surrogate light chain sequence.
5. The multi-specific SVD binding protein of claim 1, wherein the C-terminus of said second heavy chain variable domain sequence is conjugated to the N-terminus of said first surrogate light chain sequence.
6. The multi-specific SVD binding protein of claim 1, wherein said first heavy chain variable domain sequence and said first surrogate light chain sequence together form a first binding domain specifically binding to a first target.
7. The multi-specific SVD binding protein of claim 1 or claim 6, wherein said second heavy chain variable domain sequence and said second surrogate light chain sequence and together form a second binding domain specifically binding to a second target.
8. The multi-specific SVD binding protein of any one of claims 1 to 7, wherein said first and said second surrogate light chain sequences are identical.
9. The multi-specific SVD binding protein of any one of claims 1 to 7, wherein said first and said second surrogate light chain sequences are different.
10. The multi-specific SVD binding protein of claim 8 or claim 9 wherein said first and said second surrogate light chain sequences comprise a VpreB sequence.
11. The multi-specific SVD binding protein of claim 10 wherein said second surrogate light chain sequence further comprises a λ5 sequence.
12. The multi-specific SVD binding protein of claim 1, wherein the association is covalent and/or non-covalent.
13. The multi-specific SVD binding protein of any one of claims 1 to 12, wherein the conjugation is by a linker sequence.
14. The multi-specific SVD binding protein of claim 13, wherein the linker sequence is heterologous linker sequence.
15. The multi-specific SVD binding protein of any one of claims 1 to 12, wherein the conjugation is direct fusion.
16. The multi-specific SVD binding protein of claim 13, wherein the linker sequence comprises a sequence selected from the group consisting of: an antibody J region sequence, a λ5 sequence, a λ light chain constant region sequence, a κ light chain constant region sequence, synthetic sequence, and any combination thereof.
17. The multi-specific SVD binding protein of claim 16, wherein the synthetic sequence is (Gly-Gly-Gly-Ser)ₙ (SEQ ID NO:109), (Gly-Gly-Gly-Gly-Ser)ₙ (SEQ ID NO: 110), or Gly-Ala, wherein n is at least 1.
18. The multi-specific SVD binding protein of claim 15, wherein the C-terminus of the first antibody heavy chain variable domain sequence is fused to the N-terminus of the second surrogate light chain sequence forming a first polypeptide chain.
19. The multi-specific SVD binding protein of claim 15, wherein the C-terminus of the second antibody heavy chain variable domain sequence is fused to the N-terminus of the first surrogate light chain sequence forming a second polypeptide chain.
20. The multi-specific SVD binding protein of claim 19, wherein the C-terminus of the first surrogate light chain sequence is fused to the N-terminus of an antibody heavy chain constant domain sequence.
21. The multi-specific SVD binding protein of claim 20, wherein said heavy chain variable domain sequence comprises a CH1 sequence and/or an Fc region.
22. A multi-specific SVD binding protein as substantially described herein with reference to and as illustrated by any of the accompanying drawings.

### BRIEF DESCRIPTION OF THE DRAWINGS

Figures IA-F depict exemplary structures of hetero-tetrameric binding proteins, called Stacked Variable Domain (SVD) Surroglobulins, with two binding specificities. The structures of FIGS. 1A-E contain λ-like surrogate light chain sequences. The structure of FIG. 1F contains κ-like surrogate light chain sequences.

FIGS. 2A-B depict exemplary structures of hetero-tetrameric bispecific antibody molecules, called Stacked Variable Domain (SVD) Antibodies.

FIG. 3 is a schematic illustration of a surrogate light chain fusion fog used by VpreB and λ5 sequences, illustrative fusion polypeptides comprising surrogate light chain sequences, and a classic recombined antibody light chain structure derived from V-J joining.

FIG. 4 is a schematic illustration of various surrogate light chain dimeric and single chain constructs.

FIG. 5 shows the human VpreB 1 amino acid sequence of SEQ ID NO: 1 with a native leader sequence; the mouse VpreB2 sequences of SEQ ID NOS: 2 and 3; the human VpreB3-like sequence of SEQ ID NO: 4, the sequence of the truncated VpreB 1 sequence in the “trimer” designated as “VpreB dTail” (SEQ ID NO: 5); and the human VpreB1 amino acid sequence with a murine Ig κ leader sequence (SEQ ID NO:6). Underlining indicates the leader sequences within the VpreB amino acid sequences.

FIG. 6 shows the murine λ5-like sequence of SEQ ID NO: 7; the human λ5-like sequence of SEQ ID NO: 8; the sequence of the truncated λ5 sequence designated as “λ5 dTail” (SEQ ID NO: 9); and the human λ5 dTail sequence with a murine Ig κ leader sequence (SEQ ID NO: 10). Underlining indicates the leader sequences within the λ5 amino acid sequences.

FIG. 7 shows human VpreB1-λ5 chimeric amino acid sequences with a murine Ig κ leader sequence underlined (SEQ ID NOS:35 and 36).

FIGS. 8A and 8B show (A) the human Vκ-like nucleotide sequence of SEQ ID NO:11 and the amino acid sequence of the encoded protein (AJ004956; SEQ ID NO:12) (native leader sequence underlined), and (B) the predicted mature amino acid sequences of Vκ-like proteins possible from all Vκ families, each bearing different lengths of extensions (SEQ ID NOS: 13-24) aligned with AJ004956 Vκ-like prototype sequence (SEQ ID NO:12).

FIGS. 9A-C show (A) the human JCκ nucleotide sequence of SEQ ID NO:25 and the amino acid sequence of the encoded protein (SEQ ID NO:26) (unique sequence compared to predicted mature JCk proteins is doubly underlined and potential leader cleavage sequence singly underlined), (B) the predicted JCκ-like amino acid sequences from the remaining kappa J-constant region rearrangements (J1-J5Cκ) (SEQ ID NOS:27-31), and (C) the JCk engineered secretion optimized variants, including JCκ with an appended murine Ig κ leader sequence underlined (SEQ ID NO:32), a recombined JCκ only with an appended murine Ig κ leader sequence underlined (SEQ ID NO:33), and a predicted processed JCκ with an appended murine Ig κ leader sequence underlined (SEQ ID NO:34).

FIG. 10A-C show amino acid sequences of multispecific Surrobody polypeptides with λ-like surrogate light chain domains (A: SEQ ID NOS: 37-46 and 152-153; B: SEQ ID NOS: 47-55, 200-202 and 154-161; C: SEQ ID NO: 56).

FIG. 11A-C shows the amino acid sequence of a multispecific Surrobody polypeptide with λ-like surrogate light chain domains for use in a cross-complemented structure format (A: SEQ ID NOS: 57-60; B: SEQ ID NOS: 61-64; C: SEQ ID NOS: 65).

FIG. 12 shows bispecific SgGs with various lengths of linkers.

FIG. 13 compares the binding of a bispecific SVD Surrobody with binding specificities for ErbB3 and hepatocyte growth factor (HGF) to the HGF binding of scSv SgG.

FIG. 14 shows screening linker combination for target-binding using normalized transfected supernatants.

FIG. 15 shows binding affinities of two piece dual variable domain Surrobodies with binding specificities for HGF and GF.

FIG. 16 shows binding affinities of bispecific anti-VEGF/ErbB3 SVD Surrobodies.

FIG. 17 is a schematic illustration of further single chain stacked variable domain structures, including a monomeric monovalent binding and a bivalent avid binder structure.

FIG. 18 is a schematic illustration of the structure of a monomeric Stacked Variable Domain (SVD) Surrobody.

FIG. 19 is a schematic illustration of a trispecific Stacked Variable Domain (SVD) Surrobody.

FIG. 20 demonstrates that a bispecific Surrobody targeting two growth factor receptors more potently inhibits tumor cell growth than the combination of parental monospecific molecules.

FIG. 21 is a schematic illustration of a bispecific Surrobody (2-Piece) cross-complemented Stacked Variable Domains (SVD). This format does not occlude the amino terminus of either VH domain and maintains both polypeptide chains as SCL fusions.

FIG. 22A-D demonstrate inhibition of VEGF stimulated HUVEC proliferation by SVD Surrobodies as compared to a parental VEGF Surrobody.

FIG. 23 demonstrates that SVD Surrobodies inhibit neuregulin-stimulated BxPC-3 proliferation.

### DETAILED DESCRIPTION OF THE INVENTION

The present invention relates to multispecific binding molecules that can bind to two or more antigens. In particular, the invention provides Stacked Variable Domain (SVD) Surrobody and antibody molecules, as well as polypeptide chains, nucleic acids, recombinant expression vectors, host cells, and methods for making such SVD molecules. Also provided are pharmaceutical compositions containing the molecules and therapeutic or diagnostic methods using the same.

### A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provides one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

Throughout this application, the use of singular includes the plural unless expressly stated otherwise.

In this application, the use of “or” includes “and/or”, unless expressly stated otherwise.

Furthermore, the terms, “include,” “including,” and “included,” are not limiting.

In the context of the present invention, the term “antibody” (Ab) is used to refer to a native antibody from a classically recombined heavy chain derived from V(D)J gene recombination and a classically recombined light chain also derived from VJ gene recombination, or a fragment thereof.

The term “Stacked Variable Domain,” or “SVD,” in the broadest sense, is used to refer to tandem arrangements in which variable domain sequences from two different sources are conjugated to each other. In one embodiment, the conjugation takes place by direct fusion. In another embodiment, the conjugation is provided by covalent linkage through a linker sequence, such as, for example, a short peptide sequence. The reference to two different sources does not mean, however, that the variable domain sequence have to be obtained from the source from which they derive. The variable domain sequences and the tandem arrangements can be produced by any means, such as recombinant methods and/or chemical synthesis. The terms “Stacked Variable Domain” or “SVD” specifically include multi-specific (e.g. bispecific, trispecific, etc.) Surrobody- or antibody-based polypeptides comprising at least one “outer binding domain” and at least one “inner binding domain”, each specifically binding to a different target. The term specifically includes bispecific, trispecific, and other multi-specific constructs, where the variable domains may be present (“stacked”) in a single polypeptide chain (“single-chain stacked variable domains”) or two or more polypeptide chains. Thus, the terms specifically include, without limitation, monomeric, dimeric and tetrameric structures, and monovalent bispecific and bivalent bispecific structures.

The term “surrogate light chain polypeptide” or “SLC polypeptide” is used herein to refer to a VpreB polypeptide, a 2\0.5 polypeptide, a Vκ-like polypeptide, a JCκ polypeptide, and variants thereof.

The term “surrogate light chain sequence” or “SLC sequence” is used herein to refer to amino acid sequences from a native-sequence or variant VpreB polypeptide, a λ5 polypeptide, a Vκ-like polypeptide, and/or a JCκ polypeptide. SLC sequences specifically include amino acid sequences from isoforms, including splice variants and variants formed by posttranslational modifications, other mammalian homologues thereof, as well as variants of one or more of such native sequence polypeptides.

In one embodiment, the surrogate light chain sequence is a “heterologous amino acid sequence”, e.g., relative to a VpreB, as defined herein, which contemplates a VpreB sequence conjugated to (e.g., fused), or covalently associated with, a light chain constant domain region sequence (λ or κ). In another embodiment, the C-terminus of the VpreB sequence is conjugated to (e.g., fused), or covalently associated with, to the N-terminus of the light chain constant domain region sequence.

In one additional embodiment, the surrogate light chain sequence is a “heterologous amino acid sequence”, e.g., relative to a λ5, as defined herein, which contemplates a λ5 sequence conjugated (e.g. fused to), or covalently associated with, a light variable domain region sequence (λ or κ). In another embodiment, the N-terminus of the λ5 sequence is conjugated to (e.g., fused), or covalently associated with, to the C-terminus of the light chain variable domain region sequence.

In other embodiment, the surrogate light chain sequence comprises an amino acid sequence from at least two different types of surrogate light chain polypeptides. In a preferred embodiment, the surrogate light chain sequence comprises a VpreB amino acid sequence and a λ5 amino acid sequence.

The term “VpreB” is used herein in the broadest sense and refers to any native sequence or variant VpreB polypeptide, specifically including, without limitation, human VpreB1 of SEQ ID NO: 1, mouse VpreB2 of SEQ ID NOS: 2 and 3, human VpreB3-like sequence of SEQ ID NO: 4, human VpreB dT of SEQ ID NO:5, and their isoforms, including splice variants and variants formed by posttranslational modifications, other mammalian homologues thereof, as well as variants of such native sequence polypeptides. In one embodiment, VpreB is the human VpreB1 amino acid sequence with a murine Ig κ leader sequence (SEQ ID NO: 6).

The term “λ5” is used herein in the broadest sense and refers to any native sequence or variant λ5 polypeptide, specifically including, without limitation, murine λ5 of SEQ ID NO: 7, human λ5-like protein of SEQ ID NO: 8, the human λ5 dT shown as SEQ ID NO: 9, and their isoforms, including splice variants and variants formed by posttranslational modifications, other mammalian homologous thereof, as well a variants of such native sequence polypeptides. In one embodiment, λ5 is the human λ5 dTail sequence with a murine Ig κ leader sequence (SEQ ID NO:10).

The teens “variant VpreB polypeptide” and “a variant of a VpreB polypeptide” are used interchangeably, and are defined herein as a polypeptide differing from a native sequence VpreB polypeptide at one or more amino acid positions as a result of an amino acid modification. The “variant VpreB polypeptide,” as defined herein, will be different from a native antibody λ or κ light chain sequence, or a fragment thereof. The “variant VpreB polypeptide” will preferably retain at least about 65%, or at least about 70%, or at least about 75%, or at least about 80%, or at least about 85%, or at least about 90%, or at least about 95%, or at least about 98% sequence identity with a native sequence VpreB polypeptide. In another preferred embodiment, the “variant VpreB polypeptide” will be less than 95%, or less than 90%, or less than 85%, or less than 80%, or less than 75%, or less than 70%, or less than 65%, or less than 60% identical in its amino acid sequence to a native antibody λ or κ light chain sequence. Variant VpreB polypeptides specifically include, without limitation, VpreB polypeptides in which the non-Ig-like unique tail at the C-terminus of the VpreB sequence is partially or completely removed.

The terms “variant λ5 polypeptide” and “a variant of a λ5 polypeptide” are used interchangeably, and are defined herein as a polypeptide differing from a native sequence λ5 polypeptide at one or more amino acid positions as a result of an amino acid modification. The “variant λ5 polypeptide,” as defined herein, will be different from a native antibody λ or κ light chain sequence, or a fragment thereof. The “variant λ5 polypeptide” will preferably retain at least about 65%, or at least about 70%, or at least about 75%, or at least about 80%, or at least about 85%, or at least about 90%, or at least about 95%, or at least about 98% sequence identity with a native sequence λ5 polypeptide. In another preferred embodiment, the “variant λ5 polypeptide” will be less than 95%, or less than 90%, or less than 85%, or less than 80%, or less than 75%, or less than 70%, or less than 65%, or less than 60% identical in its amino acid sequence to a native antibody λ or κ light chain sequence. Variant λ5 polypeptides specifically include, without limitation, λ5 polypeptides in which the unique tail at the N-terminus of the λ5 sequence is partially or completely removed.

The terms “variant Vκ-like polypeptide” and “a variant of a Vκ-like polypeptide” are used interchangeably, and are defined herein as a polypeptide differing from a native sequence Vκ-like polypeptide at one or more amino acid positions as a result of an amino acid modification. The “variant Vκ-like polypeptide,” as defined herein, will be different from a native antibody λ or κ light chain sequence, or a fragment thereof. The “variant Vκ-like polypeptide” will preferably retain at least about 65%, or at least about 70%, or at least about 75%, or at least about 80%, or at least about 85%, or at least about 90%, or at least about 95%, or at least about 98% sequence identity with a native sequence Vκ-like polypeptide. In another preferred embodiment, the “variant Vκ-like polypeptide” will be less than 95%, or less than 90%, or less than 85%, or less than 80%, or less than 75%, or less than 70%, or less than 65%, or less than 60% identical in its amino acid sequence to a native antibody λ or κ light chain sequence. Variant Vκ-like polypeptides specifically include, without limitation, Vκ-like polypeptides in which the non-Ig-like unique tail at the C-terminus of the Vκ-like sequence is partially or completely removed.

The terms “variant JCκ polypeptide” and “a variant of a JCκ polypeptide” are used interchangeably, and are defined herein as a polypeptide differing from a native sequence JCκ polypeptide at one or more amino acid positions as a result of an amino acid modification. The “variant JCκ polypeptide,” as defined herein, will be different from a native antibody λ or κ light chain sequence, or a fragment thereof. The “variant JCκ polypeptide” will preferably retain at least about 65%, or at least about 70%, or at least about 75%, or at least about 80%, or at least about 85%, or at least about 90%, or at least about 95%, or at least about 98% sequence identity with a native sequence JCκ polypeptide. In another preferred embodiment, the “variant JCκ polypeptide” will be less than 95%, or less than 90%, or less than 85%, or less than 80%, or less than 75%, or less than 70%, or less than 65%, or less than 60% identical in its amino acid sequence to a native antibody λ or κ light chain sequence. Variant JCκ polypeptides specifically include, without limitation, JCκ polypeptides in which the unique tail at the N-terminus of the JCκ sequence is partially or completely removed.

Percent amino acid sequence identity may be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

The term “VpreB sequence” is used herein to refer to the sequence of “VpreB,” as hereinabove defined, or a fragment thereof.

The term “λ5 sequence” is used herein to refers to the sequence of “λ5,” as hereinabove defined, or a fragment thereof.

The term “Vκ-like sequence” is used herein to refer to the sequence of “Vκ-like,” as hereinabove defined, or a fragment thereof.

The term “JCκ sequence” is used herein to refer to the sequence of “JCκ,” as hereinabove defined, or a fragment thereof.

The term “λ-like surrogate light chain,” as used herein, refers to a dimer formed by the non-covalent association of a VpreB and a λ5 protein.

The term “κ-like surrogate light chain,” as used herein, refers to a dimer formed by the non-covalent association of a Vκ-like and a JCκ protein.

The term “λ-like surrogate light chain sequence,” as defined herein, means any polypeptide sequence that comprises a “VpreB sequence” and/or a “λ5 sequence,” as hereinabove defined. The “λ-like surrogate light chain sequence,” as defined herein, specifically includes, without limitation, the human VpreB1 sequence of SEQ ID NO 1, the mouse VpreB2 sequences of SEQ ID NOS: 2 and 3, and the human VpreB3 sequence of SEQ ID NO: 4, the human VpreB dT shown as SEQ ID NO: 5; and the human VpreB1 amino acid sequence of SEQ ID NO:6 and their various isoforms, including splice variants and variants formed by posttranslational modifications, homologues thereof in other mammalian species, as well as fragments and variants thereof. The term “λ-like surrogate light chain sequence” additionally includes, without limitation, the murine λ5 sequence of SEQ ID NO: 7, the human λ5-like sequence of SEQ ID NO: 8, the human λ5 dTail shown as SEQ ID NO: 9, the human λ5 dTail sequence of SEQ D NO: 10 and their isoforms, including splice variants and variants formed by posttranslational modifications, homologues thereof in other mammalian species, as well as fragments and variants thereof. The term “λ-like surrogate light chain sequence” additionally includes a sequence comprising both VpreB and λ5 sequences as hereinabove defined.

The term “κ-like surrogate light chain sequence,” as defined herein, means any polypeptide sequence that comprises a “Vκ-like sequence” and/or a “JCκ,” as hereinabove defined. The “κ-like surrogate light chain sequence,” as defined herein, specifically includes, without limitation, the human Vκ-like sequence of any of SEQ ID NOS:12-24, and their various isoforms, including splice variants and variants formed by posttranslational modifications, homologues thereof in other mammalian species, as well as fragments and variants thereof. The term “κ-like surrogate light chain sequence” additionally includes, without limitation, the human Vκ-like sequence of any of SEQ ID NOS:12-24, the human JCκ sequence of any of SEQ ID NO:25-35, and their isoforms, including splice variants and variants formed by posttranslational modifications, homologues thereof in other mammalian species, as well as fragments and variants thereof. The term “κ-like surrogate light chain sequence” additionally includes a sequence comprising both Vκ-like and JCκ sequences as hereinabove defined.

The term “surrogate light chain construct” is used in the broadest sense and includes any and all additional heterologous components, including a heterologous amino acid sequence, nucleic acid, and other molecules conjugated to a surrogate light chain sequence, wherein “conjugation” is defined below.

A “surrogate light chain construct” is also referred herein as a “Surrobody™,” or “Surrobody” and the two terms are used interchangeably. Certain Surrobody™ λ-like surrogate light chain constructs are disclosed in Xu et al., Proc. Natl. Acad. Sci. USA 2008, 105(31):10756-61 and in PCT Publication WO 2008/118970 published on Oct. 2, 2008. Also contemplated are κ-like surrogate light chain constructs as described in U.S. Patent Publication No. 2010-0062950, and Xu et al., J. Mol. Biol. 2010, 397, 352-360, the entire disclosures of which are expressly incorporated by reference herein.

In the context of the polypeptides of the present invention, the term “heterogeneous amino acid sequence,” or “heterologous amino acid sequence” relative to a first amino acid sequence, is used to refer to an amino acid sequence not naturally associated with the first amino acid sequence, at least not in the form it is present in the surrogate light chain constructs herein. For the purposes of this application, the term “heterogeneous” is interchangeable with the term “heterologous.” Thus, a “heterologous amino acid sequence” relative to a VpreB, λ5, Vκ-like, or JCκ is any amino acid sequence not associated with native VpreB, 5, Vκ-like, or JCκ in its native environment. These include, without limitation, i) λ5 sequences that are different from those λ5 sequences that, together with VpreB, form the surrogate light chain on developing B cells, such as amino acid sequence variants, e.g. truncated and/or derivatized λ5 sequences; ii) VpreB sequences that are different from those VpreB sequences that, together with λ5, form the surrogate light chain on developing B cells, such as amino acid sequence variants, e.g. truncated and/or derivatized VpreB sequences, iii) Vκ-like sequences that are different from those Vκ-like sequences that, together with JCκ, form the κ-like surrogate light chain on developing B cells, such as amino acid sequence variants, e.g. truncated and/or derivatized Vκ-like sequences; and iv) JCκ sequences that are different from those JCκ sequences that, together with Vκ-like, form the κ-like surrogate light chain on developing B cells, such as amino acid sequence variants, e.g. truncated and/or derivatized JCκ sequences.

A “heterologous amino acid sequence” relative to a VpreB or λ5 also includes VpreB or λ5 sequences covalently associated with, e.g. fused to, a corresponding VpreB or λ5, including native sequence VpreB or λ5, since in their native environment, the VpreB and λ5 sequences are not covalently associated, e.g. fused, to each other. Similarly, a “heterologous amino acid sequence” relative to a Vκ-like or JCκ also includes Vκ-like or JCκ sequences covalently associated with, e.g. fused to, a corresponding Vκ-like or JCκ, including native sequence Vκ-like or JCκ, since in their native environment, the Vκ-like or JCκ sequences are not covalently associated, e.g. fused, to each other.

A “heterologous amino acid sequence” relative to a VpreB or Vκ-like also includes VpreB or Vκ-like sequences covalently associated with, e.g. fused to, a light chain constant domain region sequence (λ or κ), or any fragment or variant thereof, since in their native environment, the VpreB or Vκ-like and the light chain constant domain region sequences (λ or κ) are not covalently associated, e.g. fused, to each other.

A “heterologous amino acid sequence” relative to a VpreB or Vκ-like also includes VpreB or Vκ-like sequences covalently associated with, e.g. fused to, a sequence providing additional functionality (e.g., a cytokine or antibody fragment amino acid sequence), or any fragment or variant thereof, since in their native environment, the VpreB or Vκ-like and the sequence providing additional functionality are not covalently associated, e.g. fused, to each other. The antibody fragment amino acid sequence may be a single chain variable fragment (scFv).

Heterologous amino acid sequences also include, without limitation, antibody sequences, including antibody and heavy chain sequences and fragments or variants thereof, such as, for example, antibody light and heavy chain variable region sequences, and antibody light and heavy chain constant region sequences.

The terms “conjugate,” “conjugated,” and “conjugation” refer to any and all forms of covalent or non-covalent linkage, and include, without limitation, direct genetic or chemical fusion, coupling through a linker or a cross-linking agent, and non-covalent association, for example through Van der Waals forces, or by using a leucine zipper.

The term “flexible linker” is used herein to refer to any linker that is not predicted, based on its chemical structure, to be fixed in three-dimensional space in its intended context and environment.

The term “fusion” is used herein to refer to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini.

As used herein, the terms “peptide,” “polypeptide” and “protein” all refer to a primary sequence of amino acids that are joined by covalent “peptide linkages.” In general, a peptide consists of a few amino acids, typically from about 2 to about 50 amino acids, and is shorter than a protein. The term “polypeptide,” as defined herein, encompasses peptides and proteins.

A “native antibody” is heterotetrameric glycoprotein of about 150,000 daltons, composed of two identical light (L) chains and two identical heavy (H) chains. Each light chain is linked to a heavy chain by covalent disulfide bond(s), while the number of disulfide linkages varies between the heavy chains of different immunoglobulin isotypes. Each heavy and light chain also has regularly spaced intrachain disulfide bridges. Each heavy chain has, at one end, a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains, Chothia et al., J. Mol. Biol. 186:651 (1985); Novotny and Haber, Proc. Natl. Acad. Sci. U.S.A. 82:4592 (1985).

The term “variable” with reference to antibody chains is used to refer to portions of the antibody chains which differ extensively in sequence among antibodies and participate in the binding and specificity of each particular antibody for its particular antigen. Such variability is concentrated in three segments called hypervariable regions both in the light chain and the heavy chain variable domains. The more highly conserved portions of variable domains are called the framework region (FR). The variable domains of native heavy and light chains each comprise four FRs (FR1, FR2, FR3 and FR4, respectively), largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991), pages 647-669). The constant domains are not involved directly in binding an antibody to an antigen, but exhibit various effector functions, such as participation of the antibody in antibody-dependent cellular toxicity.

The term “hypervariable region” when used herein refers to the amino acid residues of an antibody which are responsible for antigen-binding. The hypervariable region comprises amino acid residues from a “complementarity determining region” or “CDR” (i.e., residues 30-36 (L1), 46-55 (L2) and 86-96 (L3) in the light chain variable domain and 30-35 (H1), 47-58 (H2) and 93-101 (H3) in the heavy chain variable domain; MacCallum et al., J Mol Biol. 262(5):732-45 (1996).

The term “framework region” refers to the art recognized portions of an antibody variable region that exist between the more divergent CDR regions. Such framework regions are typically referred to as frameworks 1 through 4 (FR1, FR2, FR3, and FR4) and provide a scaffold for holding, in three-dimensional space, the three CDRs found in a heavy or light chain antibody variable region, such that the CDRs can form an antigen-binding surface.

Depending on the amino acid sequence of the constant domain of their heavy chains, antibodies can be assigned to different classes. There are five major classes of antibodies IgA, IgD, IgE, IgG, and IgM, and several of these may be further divided into subclasses (isotypes), e.g., IgG1, IgG2, IgG3, IgG4, IgA, and IgA2. In a preferred embodiment, the immunoglobulin sequences used in the construction of the immunoadhesins of the present invention are from an IgG immunoglobulin heavy chain domain. For human immunoadhesins, the use of human IgG1 and IgG3 immunoglobulin sequences is preferred. A major advantage of using the IgG1 is that IgG1 immunoadhesins can be purified efficiently on immobilized protein A. However, other structural and functional properties should be taken into account when choosing the Ig fusion partner for a particular immunoadhesin construction. For example, the IgG3 hinge is longer and more flexible, so that it can accommodate larger “adhesin” domains that may not fold or function properly when fused to IgG1. Another consideration may be valency; IgG immunoadhesins are bivalent homodimers, whereas Ig subtypes like IgA and IgM may give rise to dimeric or pentameric structures, respectively, of the basic Ig homodimer unit. For VEGF receptor Ig-like domain/immunoglobulin chimeras designed for in vivo applications, the pharmacokinetic properties and the effector functions specified by the Fc region are important as well. Although IgG1, IgG2 and IgG4 all have in vivo half-lives of 21 days, their relative potencies at activating the complement system are different. Moreover, various immunoglobulins possess varying numbers of allotypic isotypes.

The heavy-chain constant domains that correspond to the different classes of immunoglobulins are called α, β, ε, γ, and μ, respectively.

The “light chains” of antibodies from any vertebrate species can be assigned to one of two clearly distinct types, called kappa (κ) and lambda (λ), based on the amino acid sequences of their constant domains. Any reference to an antibody light chain herein includes both κ and λ light chains.

“Antibody fragments” comprise a portion of a full length antibody, generally the antigen binding or a variable domain thereof. Examples of antibody fragments include, but are not limited to, Fab, Fab′, F(ab′)₂, scFv, and (scFv)₂ fragments.

As used herein the term “antibody binding region” refers to one or more portions of an immunoglobulin or antibody variable region capable of binding an antigen(s). Typically, the antibody binding region is, for example, an antibody light chain (VL) (or variable region thereof), an antibody heavy chain (VH) (or variable region thereof), a heavy chain Fd region, a combined antibody light and heavy chain (or variable region thereof) such as a Fab, F(ab′)₂, single domain, or single chain antibody (scFv), or a full length antibody, for example, an IgG (e.g., an IgG1, IgG2, IgG3, or IgG4 subtype), IgA1, IgA2, IgD, IgE, or IgM antibody.

The term “epitope” as used herein, refers to a sequence of at least about 3 to 5, preferably at least about 5 to 10, or at least about 5 to 15 amino acids, and typically not more than about 500, or about 1,000 amino acids, which define a sequence that by itself, or as part of a larger sequence, binds to an antibody generated in response to such sequence. An epitope is not limited to a polypeptide having a sequence identical to the portion of the parent protein from which it is derived. Indeed, viral genomes are in a state of constant change and exhibit relatively high degrees of variability between isolates. Thus the term “epitope” encompasses sequences identical to the native sequence, as well as modifications, such as deletions, substitutions and/or insertions to the native sequence. Generally, such modifications are conservative in nature but non-conservative modifications are also contemplated. The term specifically includes “mimotopes,” i.e. sequences that do not identify a continuous linear native sequence or do not necessarily occur in a native protein, but functionally mimic an epitope on a native protein. The term “epitope” specifically includes linear and conformational epitopes.

The term “amino acid” or “amino acid residue” typically refers to an amino acid having its art recognized definition such as an amino acid selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val) although modified, synthetic, or rare amino acids may be used as desired. Thus, modified and unusual amino acids listed in 37 CFR 1.822(b)(4) are specifically included within this definition and expressly incorporated herein by reference. Amino acids can be subdivided into various sub-groups. Thus, amino acids can be grouped as having a nonpolar side chain (e.g., Ala, Cys, Ile, Leu, Met, Phe, Pro, Val); a negatively charged side chain (e.g., Asp, Glu); a positively charged side chain (e.g., Arg, His, Lys); or an uncharged polar side chain (e.g., Asn, Cys, Gln, Gly, His, Met, Phe, Ser, Thr, Trp, and Tyr). Amino acids can also be grouped as small amino acids (Gly, Ala), nucleophilic amino acids (Ser, His, Thr, Cys), hydrophobic amino acids (Val, Leu, Ile, Met, Pro), aromatic amino acids (Phe, Tyr, Trp, Asp, Glu), amides (Asp, Glu), and basic amino acids (Lys, Arg).

The teen “polynucleotide(s)” refers to nucleic acids such as DNA molecules and RNA molecules and analogs thereof (e.g., DNA or RNA generated using nucleotide analogs or using nucleic acid chemistry). As desired, the polynucleotides may be made synthetically, e.g., using art-recognized nucleic acid chemistry or enzymatically using, e.g., a polymerase, and, if desired, be modified. Typical modifications include methylation, biotinylation, and other art-known modifications. In addition, the nucleic acid molecule can be single-stranded or double-stranded and, where desired, linked to a detectable moiety.

The term “variant” with respect to a reference polypeptide refers to a polypeptide that possesses at least one amino acid mutation or modification (i.e., alteration) as compared to a native polypeptide. Variants generated by “amino acid modifications” can be produced, for example, by substituting, deleting, inserting and/or chemically modifying at least one amino acid in the native amino acid sequence.

An “amino acid modification” refers to a change in the amino acid sequence of a predetermined amino acid sequence. Exemplary modifications include an amino acid substitution, insertion and/or deletion.

An “amino acid modification at” a specified position, refers to the substitution or deletion of the specified residue, or the insertion of at least one amino acid residue adjacent the specified residue. By insertion “adjacent” a specified residue is meant insertion within one to two residues thereof. The insertion may be N-terminal or C-terminal to the specified residue.

An “amino acid substitution” refers to the replacement of at least one existing amino acid residue in a predetermined amino acid sequence with another different “replacement” amino acid residue. The replacement residue or residues may be “naturally occurring amino acid residues” (i.e. encoded by the genetic code) and selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val). Substitution with one or more non-naturally occurring amino acid residues is also encompassed by the definition of an amino acid substitution herein.

A “non-naturally occurring amino acid residue” refers to a residue, other than those naturally occurring amino acid residues listed above, which is able to covalently bind adjacent amino acid residues(s) in a polypeptide chain. Examples of non-naturally occurring amino acid residues include norleucine, ornithine, norvaline, homoserine and other amino acid residue analogues such as those described in Ellman et al. Meth. Enzym. 202:301 336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA.

An “amino acid insertion” refers to the incorporation of at least one amino acid into a predetermined amino acid sequence. While the insertion will usually consist of the insertion of one or two amino acid residues, the present application contemplates larger “peptide insertions”, e.g. insertion of about three to about five or even up to about ten amino acid residues. The inserted residue(s) may be naturally occurring or non-naturally occurring as disclosed above.

An “amino acid deletion” refers to the removal of at least one amino acid residue from a predetermined amino acid sequence.

The term “mutagenesis” refers to, unless otherwise specified, any art recognized technique for altering a polynucleotide or polypeptide sequence. Preferred types of mutagenesis include error prone PCR mutagenesis, saturation mutagenesis, or other site directed mutagenesis.

“Site-directed mutagenesis” is a technique standard in the art, and is conducted using a synthetic oligonucleotide primer complementary to a single-stranded phage DNA to be mutagenized except for limited mismatching, representing the desired mutation. Briefly, the synthetic oligonucleotide is used as a primer to direct synthesis of a strand complementary to the single-stranded phage DNA, and the resulting double-stranded DNA is transformed into a phage-supporting host bacterium. Cultures of the transformed bacteria are plated in top agar, permitting plaque formation from single cells that harbor the phage. Theoretically, 50% of the new plaques will contain the phage having, as a single strand, the mutated form; 50% will have the original sequence. Plaques of interest are selected by hybridizing with kinased synthetic primer at a temperature that permits hybridization of an exact match, but at which the mismatches with the original strand are sufficient to prevent hybridization. Plaques that hybridize with the probe are then selected, sequenced and cultured, and the DNA is recovered.

The term “vector” is used to refer to a rDNA molecule capable of autonomous replication in a cell and to which a DNA segment, e.g., gene or polynucleotide, can be operatively linked so as to bring about replication of the attached segment. Vectors capable of directing the expression of genes encoding for one or more polypeptides are referred to herein as “expression vectors. “The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers. A vector may be a “plasmid” referring to a circular double-stranded DNA loop into which additional DNA segments may be ligated. A vector may be a phage vector or a viral vector, in which additional DNA segments may be ligated into the viral genome. Suitable vectors are capable of autonomous replication in a host cell into which they are introduced, e.g., bacterial vector with a bacterial origin or replication and episomal mammalian vectors. A vector may be integrated into the host cell genome, e.g., a non-episomal mammalian vector, upon introduction into the host cell, and replicated along with the host genome.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

A “phage display library” is a protein expression library that expresses a collection of cloned protein sequences as fusions with a phage coat protein. Thus, the phrase “phage display library” refers herein to a collection of phage (e.g., filamentous phage) wherein the phage express an external (typically heterologous) protein. The external protein is free to interact with (bind to) other moieties with which the phage are contacted. Each phage displaying an external protein is a “member” of the phage display library.

The term “filamentous phage” refers to a viral particle capable of displaying a heterogeneous polypeptide on its surface, and includes, without limitation, f1, fd, Pf1, and M13. The filamentous phage may contain a selectable marker such as tetracycline (e.g., “fd-tet”). Various filamentous phage display systems are well known to those of skill in the art (see, e.g., Zacher et al. Gene 9: 127-140 (1980), Smith et al. Science 228: 1315-1317 (1985); and Parmley and Smith Gene 73: 305-318 (1988)).

The term “panning” is used to refer to the multiple rounds of screening process in identification and isolation of phages carrying compounds, such as antibodies, with high affinity and specificity to a target.

A “leader sequence,” “signal peptide,” or a “secretory leader,” which terms are used interchangeably, contains a sequence comprising amino acid residues that directs the intracellular trafficking of the polypeptide to which it is a part. Polypeptides contain secretory leaders, signal peptides or leader sequences, typically at their N-terminus. These polypeptides may also contain cleavage sites where the leader sequences may be cleaved from the rest of the polypeptides by signal endopeptidases. Such cleavage results in the generation of mature polypeptides. Cleavage typically takes place during secretion or after the intact polypeptide has been directed to the appropriate cellular compartment.

A “host cell” includes an individual cell or cell culture which can be or has been a recipient for transformation of nucleic acid(s) and/or vector(s) containing nucleic acids encoding the molecules described herein. In methods of the present invention, a host cell can be a eukaryotic cell, such as a Chinese Hamster Ovary (CHO) cell, or a human embryonic kidney (HEK) 293 cell. Other suitable host cells are known to those skilled in the art.

### B. Detailed Description

Techniques for performing the methods of the present invention are well known in the art and described in standard laboratory textbooks, including, for example, Ausubel et al., Current Protocols of Molecular Biology, John Wiley and Sons (1997); Molecular Cloning: A Laboratory Manual, Third Edition, J. Sambrook and D. W. Russell, eds., Cold Spring Harbor, N.Y., USA, Cold Spring Harbor Laboratory Press, 2001; O'Brian et al., Analytical Chemistry of Bacillus Thuringiensis, Hickle and Fitch, eds., Am. Chem. Soc., 1990; Bacillus thuringiensis: biology, ecology and safety, T. R. Glare and M. O'Callaghan, eds., John Wiley, 2000; Antibody Phage Display, Methods and Protocols, Humana Press, 2001; and Antibodies, G. Subramanian, ed., Kluwer Academic, 2004. Mutagenesis can, for example, be performed using site-directed mutagenesis (Kunkel et al., Proc. Natl. Acad. Sci USA 82:488-492 (1985)). PCR amplification methods are described in U.S. Pat. Nos. 4,683,192, 4,683,202, 4,800,159, and 4,965,188, and in several textbooks including “PCR Technology: Principles and Applications for DNA Amplification”, H. Erlich, ed., Stockton Press, New York (1989); and PCR Protocols: A Guide to Methods and Applications, Innis et al., eds., Academic Press, San Diego, Calif. (1990).

1. Multispecific Stacked Variable Domain (SVD) Binding Molecules

In one embodiment, the invention concerns stacked variable domain (SVD) Surroglobulin structures, i.e. heteromeric binding proteins designed such that two domains from two different parental Surrobodies are covalently linked in tandem directly or via a designed linker. Specifically the first component of the complex is the tandem product of a heavy chain variable domain (VH) of the first surrobody and the surrogate light chain domain of a second surrobody linked together, which is intended to create the “outer” binding domain. The second component of the SVD complex is the tandem product of a surrogate light chain domain of the first surrobody and the heavy chain variable domain (VH) of a second surrobody linked together, which is intended to create the “inner” binding domain. This second component may be followed by a constant domain sequence (e.g. CH1) and, if desired, an Fc region to enable avid binding to both specificities. The two components, though typically single polypeptides, can be individual dimeric proteins.

In one aspect, the SVD molecules of the present invention may utilize different antibody heavy chain constant domain region sequences. In one embodiment, the heavy chain constant domain sequence comprises a sequence selected from the group consisting of: a CH1 sequence, a CH2 sequence, a CH3 sequence, a CH1 and a CH3 sequence, a CH2 and a CH3 sequence, an Fc region, as well as any functionally active fragment thereof.

In another embodiment, the invention concerns an SVD Surroglobulin structure, comprising a single chain product of a heavy chain variable domain (VH) of a first surrobody linked to its cognate surrogate light chain that is intended to create the “outer” binding domain, which is in turn linked to the surrogate light chain of a second surrobody. In this embodiment, the second component of the SVD complex is the heavy chain variable domain (VH) of a second surrobody, which is intended to create the “inner” binding domain. This second heavy chain may be followed by the constant domain (CH1) and if desired the Fc region for avid binding to both each distinct binding target. In this embodiment, the first binding domain specificity is created as a single chain construct fused to the surrogate light chain of a second binding specificity to restore native binding affinities of a parental Surroglobulin (SgG). However, if the second binding domain maintains native binding affinities in the presence of a fusion on the N-terminus then it is also possible to fuse the single chain construct with a similar effect.

Furthermore it is possible to fuse distinct single chain binding domains to both the amino terminus of the surrogate light chain and the amino terminus of the heavy chain to create a trispecific, avid heteromeric binding protein.

In yet another embodiment, a panel of SVD-SgG molecules are created, composed of combinations of heavy chain variable (VH) domains of neutralizing surroglobulins and combinatorial linker diversity to identify combinations with potentiated or additional activity. The beneficial combination have the potential to be generated into a more potent agent, as well as a more consistent product than a cocktail admixture of biologics, such as antibodies.

In another example of targeting a single molecule a single heavy chain variable domain (VH) is used for each of the four binding sites of an SVD-SgG construct, to create a molecule that is capable of either binding stoichiometrically larger amounts of target or creating higher order clusters of the targeted protein.

The multispecific stacked variable domain (SVD) binding molecules, as defined herein, contain different polypeptide components. The present invention contemplates the use of fragments of these polypeptide components, in particular, functional fragments. The term “fragment” refers to a portion of a polypeptide or sequence described herein, generally comprising at least the region involved in binding a target and/or in association with another polypeptide or sequence. A “functional fragment, ” as defined herein, is a portion of a polypeptide or sequence which has a qualitative biological activity in common with the original (reference) polypeptide or sequence. Thus, for example, a fragment of a surrogate light chain (SLC) polypeptide or sequence may be a functional fragment, which comprises at least a minimum sequence length required for retaining a qualitative biological activity of the SLC polypeptide or sequence. For example, the functional fragment may retain the qualitative ability to bind a target either alone or in combination with another polypeptide, e.g., an antibody heavy chain variable region sequence, and/or the ability to associate with another polypeptide, e.g., an antibody heavy chain constant region.

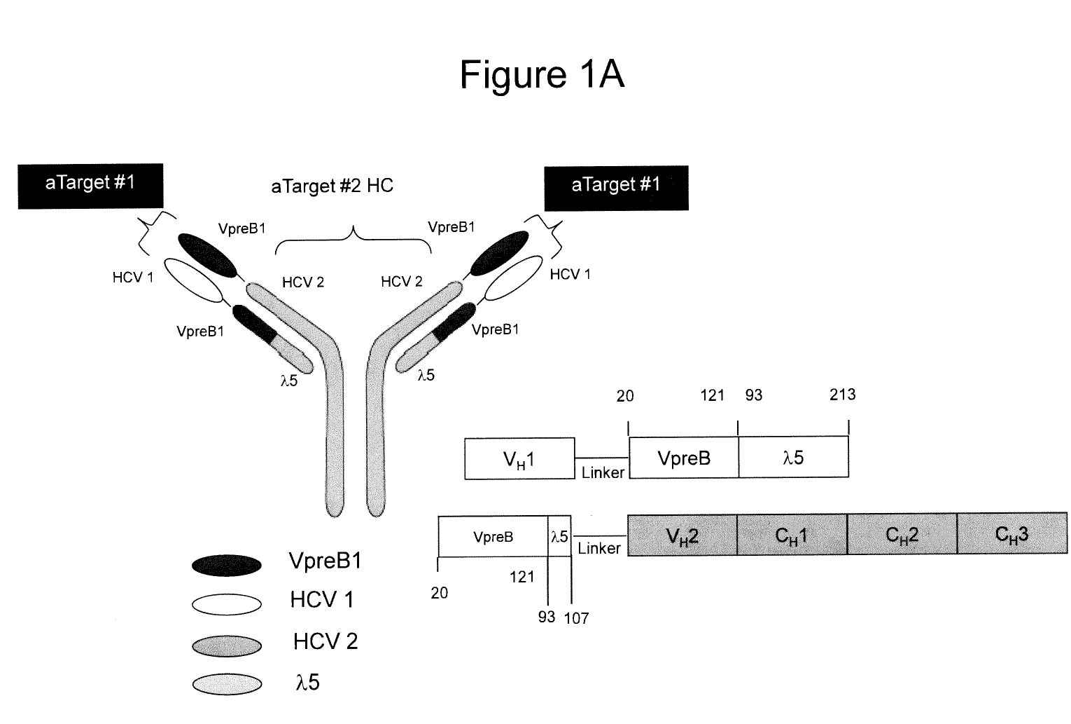

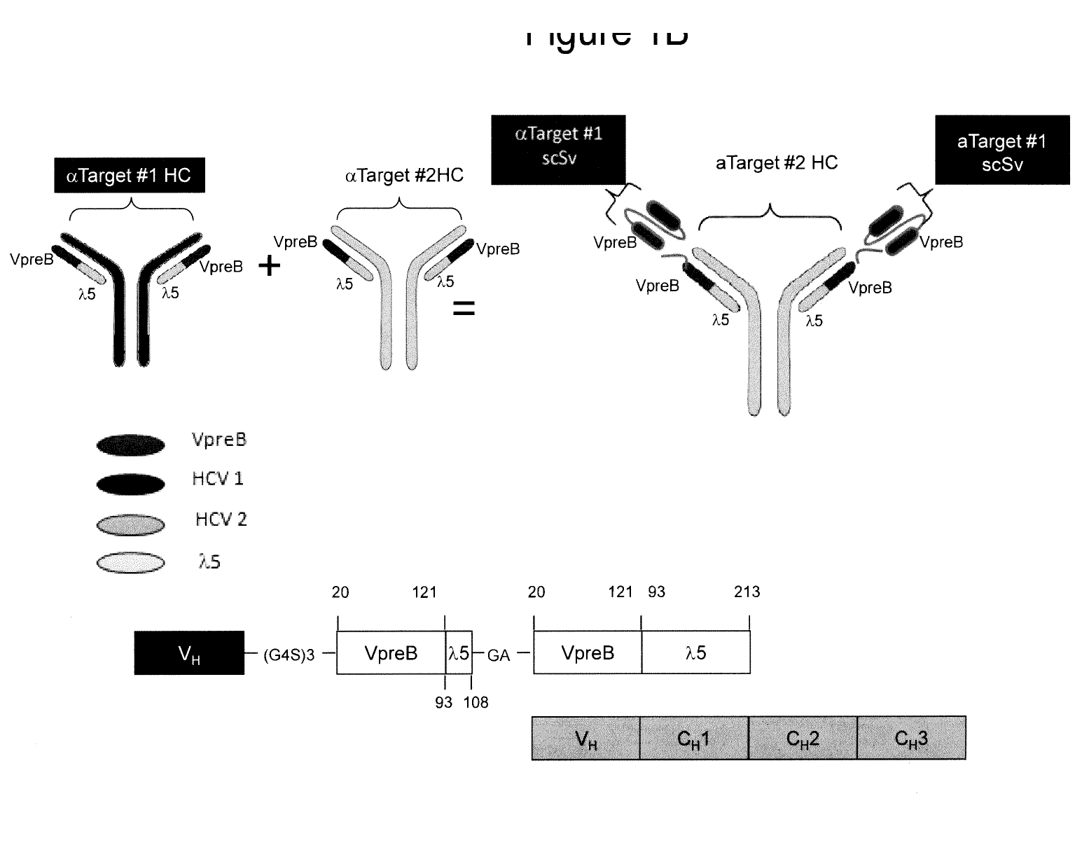

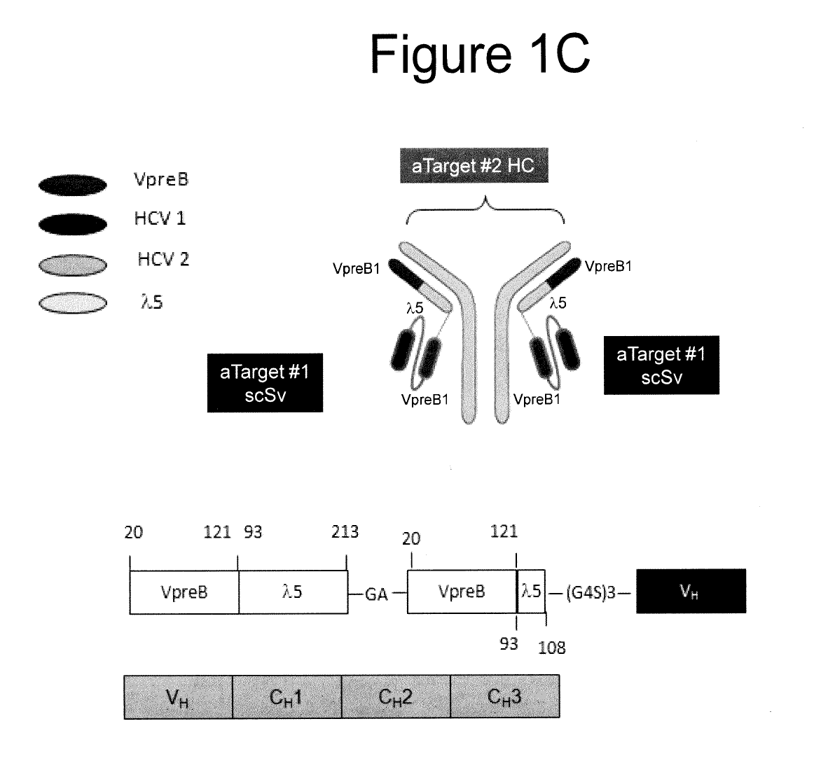

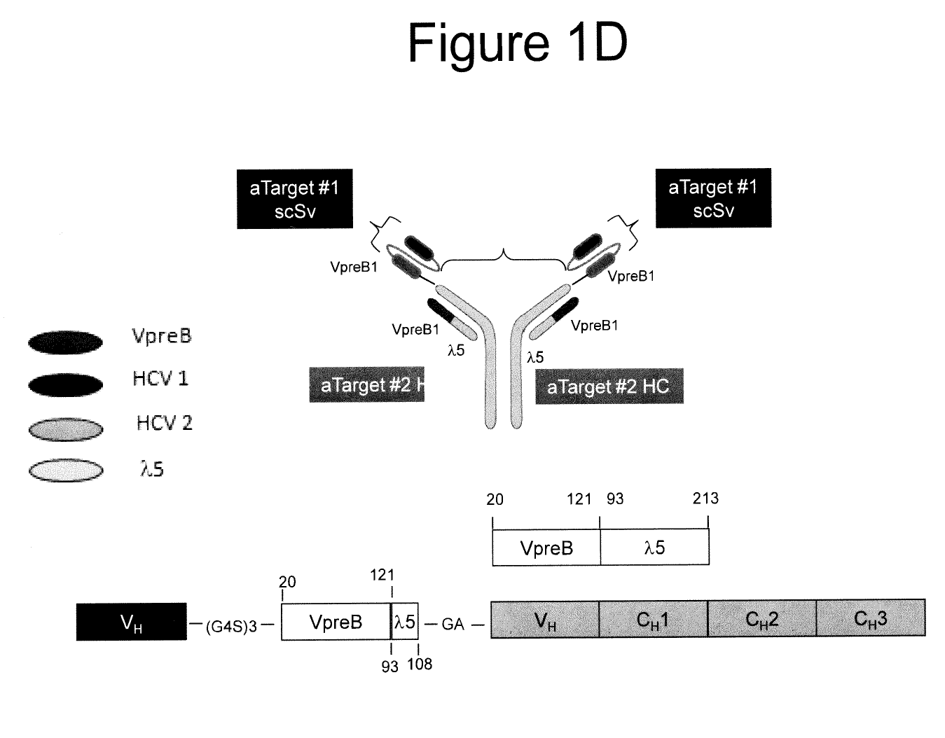

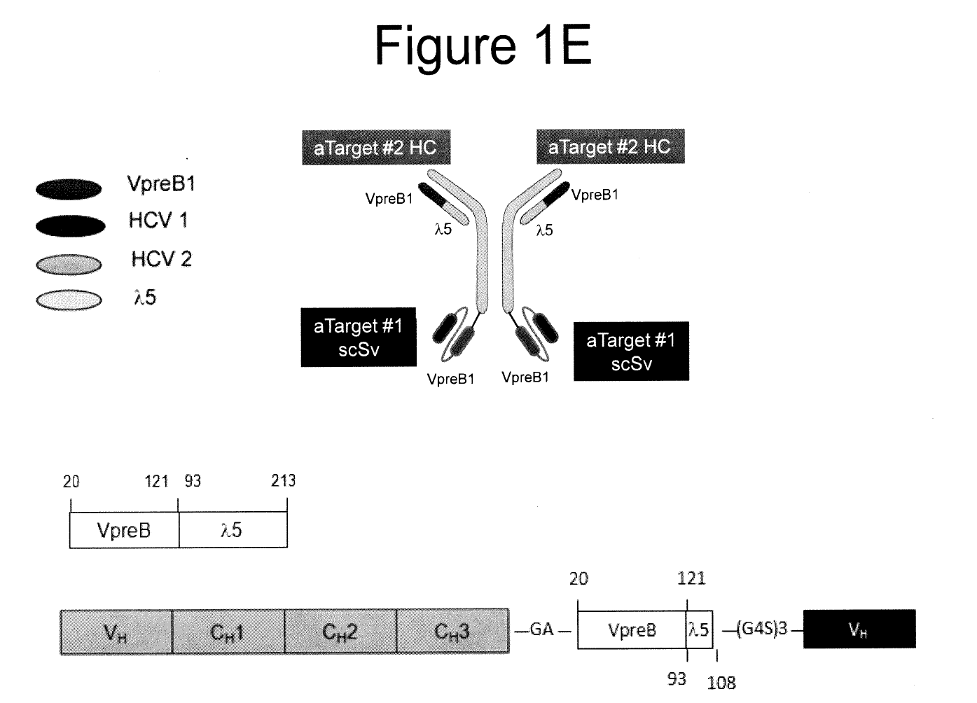

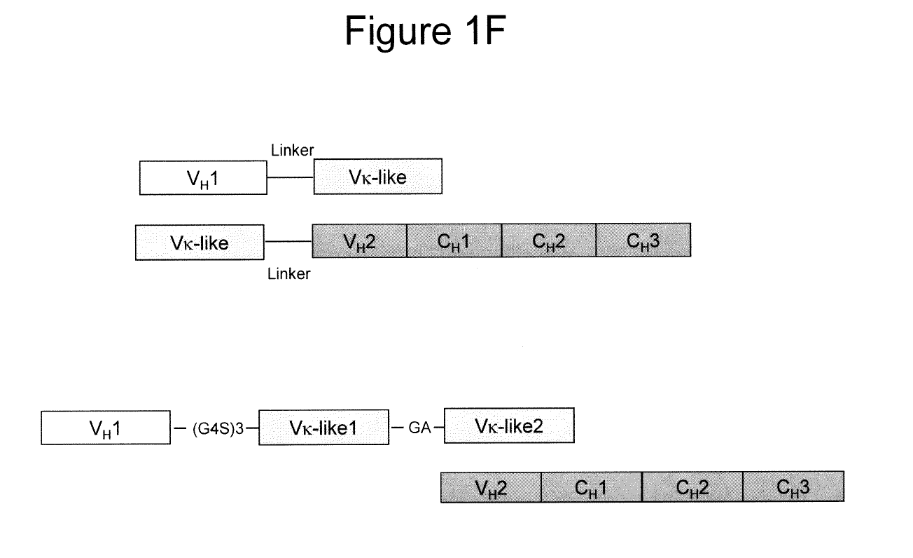

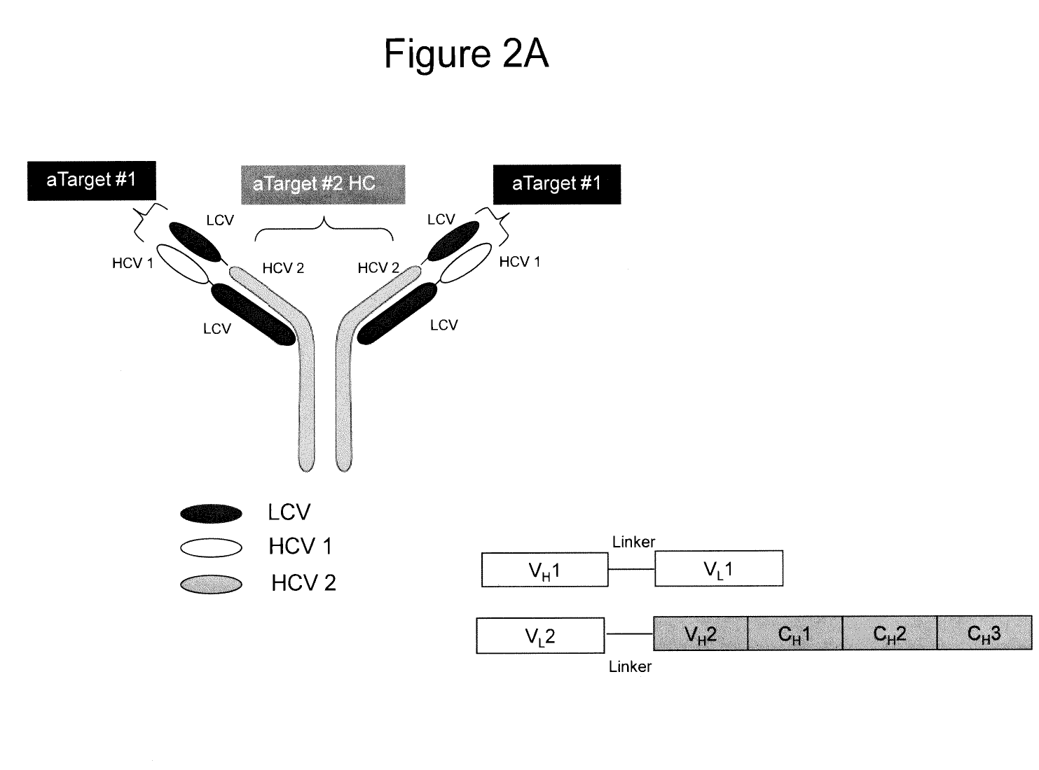

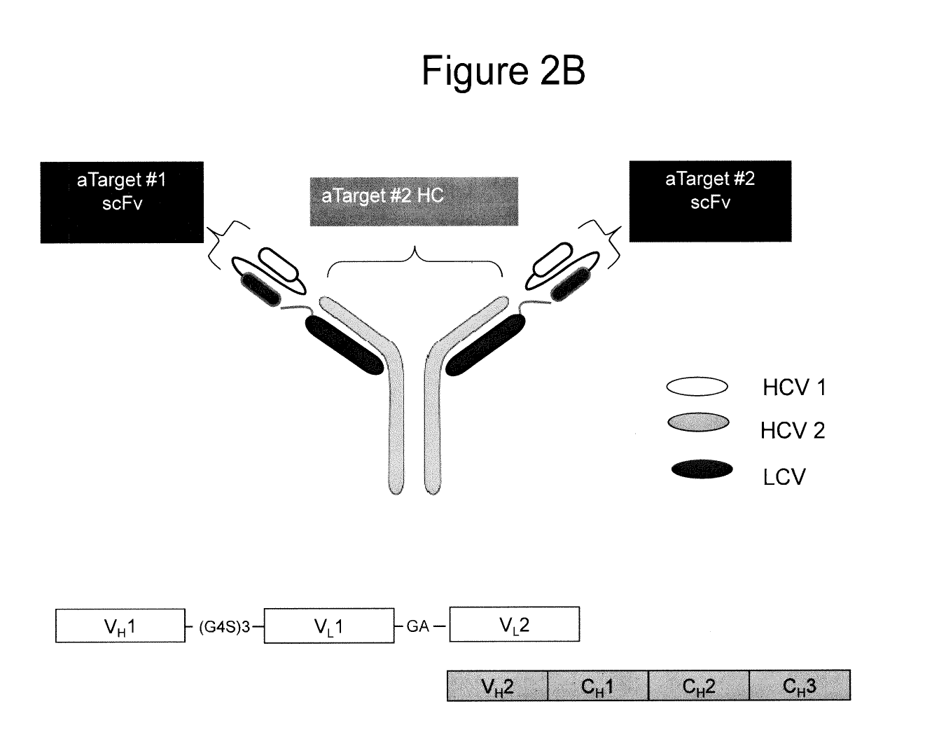

Although the multispecific stacked variable domain binding proteins or molecules described herein contain surrogate light chain sequences, such formats may be adapted for use with antibody light chain sequences and heavy chain sequences. Examples of this are provided in FIGS. 2A-B. These and further embodiments are illustrated in the Examples and associated Figures.

A. Bispecific Surrobody Structures

In one aspect, the multispecific SVD binding proteins may be provided in a bispecific Surrobody (SgG) structure format. This format contemplates polypeptide chains and heteromultimeric bispecific binding proteins. The polypeptide chains are made up of polypeptide sequences having antibody heavy chain variable (HCV) region sequences and/or surrogate light chain (SLC) sequences. In one embodiment, a first polypeptide chain is provided having a first polypeptide sequence containing an HCV sequence specific for a first target conjugated to a second polypeptide sequence containing an SLC sequence. The C-terminus of the first polypeptide sequence containing the HCV sequence may be conjugated to the N-terminus of the second polypeptide sequence containing the SLC sequence.

In another embodiment, the first polypeptide chain is associated with a second polypeptide chain. The second polypeptide chain has a first polypeptide sequence containing an SLC sequence conjugated to a second polypeptide sequence containing an antibody heavy chain that has a variable region sequence specific for a second target. The C-terminus of the first polypeptide sequence of the second polypeptide chain may be conjugated to the N-teiminus of the second polypeptide sequence containing the variable region sequence specific for the second target. A binding site for the first target (e.g., Target#1 of FIG. 1A) may be formed between the variable region sequence of the first polypeptide chain and the SLC sequence of the second polypeptide chain.

In one other embodiment, the invention provides a heteromeric bispecific binding protein that is made up of the first and second polypeptide chains. A binding site for the second target (e.g., Target#2 of FIG. 1A) may be formed between the variable region sequence specific for the second target on the second polypeptide chain and the SLC sequence of the first polypeptide chain.

In some embodiments, the SLC sequence is further conjugated to a heterologous amino acid sequence. The conjugation may occur at the C-terminus of the polypeptide sequence that contains the SLC sequence. In one embodiment, the heterologous amino acid sequence contains a sequence selected from the group consisting of a λ5 sequence, an antibody J-region sequence, a light chain constant domain region sequence, and an amino acid sequence providing additional functionality.

The conjugations between different sequences of the polypeptide chains and heteromeric bispecific binding proteins may be by a linker sequence. In one embodiment, the linker sequence is a heterogeneous linker sequence. In another embodiment, the linker sequence contains a sequence selected from the group consisting of an antibody J region sequence, an antibody constant domain region sequence, a synthetic sequence, and any combination thereof. In one other embodiment, the conjugation is a direct fusion. In yet another embodiment, the conjugation is by a linker sequence. Exemplary linker sequences are described herein. For the purposes of this application, the term “heterogeneous” is interchangeable with the term “heterologous”, where linker sequences are concerned.

In one embodiment, the association among the components of the heteromeric bispecific binding proteins, e.g., the polypeptide chains, is a covalent and/or non-covalent association.

B. Multispecific/Bispecific Single Chain-Based Surrobody (scSv) Structures

In another aspect, the multispecific SVD binding proteins may be provided in a bispecific single chain Surrobody (scSv) structure format as exemplified in FIGS. 1B, 1C, 1D, and 1E. This format contemplates polypeptide chains and heteromultimeric bispecific binding proteins. The polypeptide chains are made up of antibody heavy chain variable (HCV) region sequences and two or more surrogate light chain (SLC) sequences.

In one other aspect, the multispecific single chain-based Surrobody (scSv) structure comprises a first polypeptide chain having an scFv-based component conjugated to an SLC sequence component, where the scSV-based component is located N-terminal to the SLC sequence component (e.g., FIG. 1B). In one embodiment, a first polypeptide chain is provided comprising an antibody heavy chain variable region sequence specific for a first target, C-terminally conjugated to a first polypeptide sequence comprising a first VpreB sequence, wherein the first polypeptide sequence comprising the VpreB sequence is C-terminally conjugated to a second polypeptide sequence comprising a second VpreB sequence, conjugated to a heterologous sequence. In another embodiment, the first polypeptide chain is associated with a second polypeptide chain comprising an antibody heavy chain comprising a variable region sequence specific for a second polypeptide target. In the first polypeptide chain, the antibody heavy chain variable region sequence of the first polypeptide chain and the first VpreB sequence of the first polypeptide chain form a binding site for said first target (e.g., Target#1 scFv in FIG. 1B).

In one other embodiment, the present invention provides a heteromeric bispecific binding protein comprising two pairs of the first and second polypeptides, associated with each other. In the heteromeric bispecific binding protein, the heavy chain variable region of the second antibody heavy chain variable region sequence specific for said second target and the second VpreB sequence of the first polypeptide chain form a binding site for a second target (e.g., Target#2 in FIG. 1B). In one embodiment, the first polypeptide chain is conjugated by a linker sequence. In another embodiment, the linker sequence is a heterologous linker sequence. In yet another embodiment, the conjugation in the first polypeptide chain is direct fusion. In some embodiments, the linker sequence is between the antibody heavy chain variable region sequence and the first polypeptide sequence comprising a first VpreB sequence comprises the amino acid sequence Gly-Gly-Gly-Gly-Ser (SEQ ID NO: 108) (e.g., FIG. 1B). In another embodiment, the linker sequence between the first polypeptide sequence comprising a first VpreB sequence and the second polypeptide sequence comprising a second VpreB sequence comprises the amino acid sequence Gly-Ala (e.g., FIG. 1B).

In an additional aspect, the multispecific single chain-based Surrobody (scSv) structure comprises a first polypeptide chain having an scSv-based component conjugated to an SLC sequence component, where the scSV-based component is located C-terminal to the SLC sequence component (e.g., FIG. 1C). In one embodiment, a first polypeptide chain is provided comprising an antibody heavy chain variable region sequence specific for a first target, N-terminally conjugated to a first polypeptide sequence comprising a VpreB sequence, wherein the first polypeptide sequence comprising the VpreB sequence is N-terminally conjugated to a second polypeptide sequence comprising a second VpreB sequence. In another embodiment, the first polypeptide chain is associated with a second polypeptide chain comprising an antibody heavy chain comprising a variable region sequence specific for a second polypeptide target. In the first polypeptide chain, the antibody heavy chain variable region sequence and the VpreB sequence form a binding site for said first target (e.g., Target#1 in FIG. 1C). In one other embodiment, the present invention provides a heteromeric bispecific binding protein comprising two pairs of the first and second polypeptides, associated with each other. In the heteromeric bispecific binding protein, the heavy chain variable region of the second antibody heavy chain variable region sequence specific for said second target and the VpreB sequence of the first polypeptide chain form a binding site for a second target (e.g., Target#2 in FIG. 1C). In one embodiment, the first polypeptide chain is conjugated by a linker sequence. In another embodiment, the linker sequence is a heterologous linker sequence. In yet another embodiment, the conjugation in the first polypeptide chain is direct fusion. In some embodiments, the linker sequence is between the antibody heavy chain variable region sequence and the first polypeptide sequence comprising a first VpreB sequence comprises the amino acid sequence Gly-Gly-Gly-Gly-Ser (SEQ ID NO: 108) (e.g., FIG. 1C). In another embodiment, the linker sequence between the first polypeptide sequence comprising a first VpreB sequence and the second polypeptide sequence comprising a second VpreB sequence comprises the amino acid sequence Gly-Ala (e.g., FIG. 1C).

In another aspect, the multispecific single chain-based Surrobody (scSv) structure comprises a first polypeptide chain having an scSv-based component conjugated to an antibody HC variable domain region component, where the scSV-based component is located N-terminal to the antibody HC variable domain region component (e.g., FIG. 1D). In one embodiment, a first polypeptide chain is provided comprising a first antibody heavy chain (HC) variable region sequence specific for a first target, C-terminally conjugated to a first polypeptide sequence comprising a first VpreB sequence, wherein the first polypeptide sequence comprising the VpreB sequence is C-terminally conjugated to a second polypeptide sequence comprising a second antibody HCV region sequence specific for a second target. In another embodiment, the second antibody HC variable region sequence further comprises an antibody heavy chain constant domain sequence. In one embodiment, the N-terminus of the antibody HC constant domain sequence is conjugated to the C-terminus of the second antibody HC variable region sequence. In some embodiments, the antibody HC constant domain sequence comprises a CH1 sequence and/or an Fc region. In another embodiment, the first polypeptide chain is associated with a second polypeptide chain comprising a second VpreB sequence, conjugated to a heterologous sequence. In the first polypeptide chain, the first antibody heavy chain variable region sequence and the first VpreB sequence of the first polypeptide chain form a binding site for said first target (e.g., Target#1 in FIG. 1D). In one other embodiment, the present invention provides a heteromeric bispecific binding protein comprising two pairs of the first and second polypeptides, associated with each other. In the heteromeric bispecific binding protein, the second antibody HC variable region sequence specific for the second target on the first polypeptide chain and the second VpreB sequence of the second polypeptide chain form a binding site for a second target (e.g., Target#2 in FIG. 1D). In one embodiment, the first polypeptide chain is conjugated by a linker sequence. In another embodiment, the linker sequence is a heterologous linker sequence. In some embodiments, the linker sequence between the antibody heavy chain variable region sequence and the first polypeptide sequence comprising the VpreB sequence comprises the amino acid sequence Gly-Gly-Gly-Gly-Ser (SEQ ID NO: 108) (e.g., FIG. 1D). In one other embodiment, the linker sequence between the first polypeptide sequence comprising the first VpreB sequence and the second polypeptide sequence comprising a second antibody HCV region sequence comprises the amino acid sequence Gly-Ala (e.g., FIG. 1D). In yet another embodiment, the conjugation in the first polypeptide chain is direct fusion.

In one additional aspect, the multispecific single chain-based Surrobody (scSv) structure comprises a first polypeptide chain having an scSv-based component conjugated to an antibody HC variable domain region component, where the scSV-based component is located C-terminal to the antibody HC variable domain region component (e.g., FIG. 1E).

In one embodiment, a first polypeptide chain is provided comprising a first antibody heavy chain (HC) variable region sequence specific for a first target, N-terminally conjugated to a first polypeptide sequence comprising a first VpreB sequence, wherein the first polypeptide sequence comprising the VpreB sequence is N-terminally conjugated to a second polypeptide sequence comprising a second antibody HC variable region sequence specific for a second target. In another embodiment, the first antibody HC variable region sequence further comprises an antibody heavy chain constant domain sequence. In one embodiment, the N-terminus of the antibody HC constant domain sequence is conjugated to the C-terminus of the first antibody ETC variable region sequence and the C-terminus of the antibody HC constant domain sequence is conjugated to the N-terminus of the first polypeptide sequence comprising the first VpreB sequence. In some embodiments, the antibody HC constant domain sequence comprises a CH1 sequence and/or an Fc region. In another embodiment, the first polypeptide chain is associated with a second polypeptide chain comprising a second VpreB sequence, conjugated to a heterologous sequence. In the first polypeptide chain, the first antibody heavy chain variable region sequence and the VpreB sequence of the first polypeptide sequence form a binding site for the first target (e.g., Target#1 in FIG. 1E). In an additional embodiment, the present invention provides a heteromeric bispecific binding protein comprising two pairs of the first and second polypeptides, associated with each other. In the heteromeric bispecific binding protein, the second antibody HC variable region sequence specific for the second target on the first polypeptide chain and the second VpreB sequence of the second polypeptide chain form a binding site for a second target (e.g., Target#2 in FIG. 1E). In one embodiment, the first polypeptide chain is conjugated by a linker sequence. In another embodiment, the linker sequence is a heterologous linker sequence. In some embodiments, the linker sequence between the first antibody heavy chain variable region sequence and the first polypeptide sequence comprising a first VpreB sequence comprises the amino acid sequence Gly-Gly-Gly-Gly-Ser (SEQ ID NO: 108) (e.g., FIG. 1E). In one other embodiment, the linker sequence between the first polypeptide sequence comprising a VpreB sequence and the second polypeptide sequence comprising a second antibody HCV region sequence comprises the amino acid sequence Gly-Ala (e.g., FIG. 1E).

In one embodiment, the association among the components of the heteromeric bispecific binding proteins, e.g., the polypeptide chains, is a covalent and/or non-covalent association.

In another embodiment, the VpreB sequence is fused, at its C-terminus, to a heterologous sequence. The heterogenous sequence is selected from the group consisting of a λ5 sequence and a light chain constant domain region sequence.

### C. Monomeric Binder Surrobody Structures

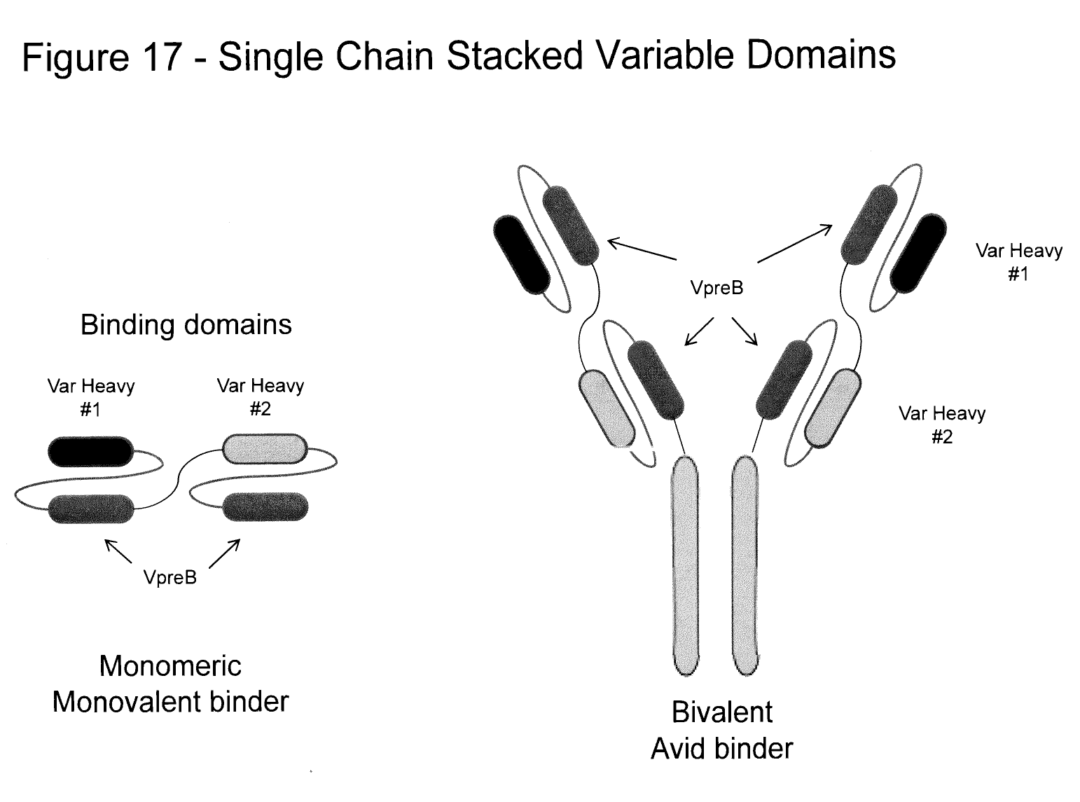

In another aspect, the multispecific SVD binding proteins may be provided in a monomeric monovalent binder or bivalent avid binder Surrobody structure format, as exemplified in FIG. 17. This format contemplates polypeptide chains and heteromultimeric bispecific binding proteins. The polypeptide chains are made up of antibody heavy chain variable (HCV) region sequences, two or more surrogate light chain (SLC) sequences, and dimerization domains. In one embodiment, the present invention provides a polypeptide chain comprising an antibody heavy chain variable region sequence specific for a first target, C-terminally conjugated to a first polypeptide sequence comprising a first surrogate light chain (SLC) sequence, wherein the first SLC sequence is C-terminally conjugated to an antibody heavy chain variable region sequence specific for a second target. In the polypeptide chain, the antibody heavy chain variable region sequence specific for a second target is C-terminally conjugated to a second surrogate light chain (SLC) sequence. In one embodiment, the second SLC sequence is conjugated to a dimerization domain. In another embodiment, the dimerization domain comprises an antibody constant domain. In one other embodiment, the dimerization domain comprises an Fc region.

In another embodiment, the present invention provides a multimeric bispecific binding protein of comprising one pair of the polypeptides. In the polypeptides or multimeric bispecific binding proteins, the heavy chain variable region specific for the first target and the first SLC sequence form a binding site for the first target. In one other embodiment, the heavy chain variable region specific for the second target and the second SLC sequence form a binding site for the second target.

In one embodiment, the conjugation in the polypeptide chains or multimeric bispecific binding proteins is by a linker sequence. In another embodiment, the linker sequence is a heterologous linker sequence. In some embodiments, the conjugation in the polypeptide chains or multimeric bispecific binding proteins is by direct fusion. In one other embodiment, the linker sequence comprises a sequence selected from the group consisting of: an antibody J region sequence, a λ5 sequence, a λ light chain constant region sequence, a κ light chain constant region sequence, synthetic sequence, and any combination thereof. In yet another embodiment, the the SLC sequence in the polypeptide chain or multimeric bispecific binding protein comprises a VpreB sequence.

### D. Bispecific Monomeric Stacked Variable Domain Surrobody Structures

In another aspect, the multispecific SVD binding proteins may be provided in a bispecific monomeric stacked variable domain Surrobody structure format where the N-terminus of the dimerization domain is utilized for conjugation, as exemplified in FIG. 18. This format contemplates polypeptide chains and heteromultimeric bispecific binding proteins. The polypeptide chains are made up of antibody heavy chain variable (HCV) region sequences, two or more surrogate light chain (SLC) sequences, and dimerization domains. In one embodiment, the present invention provides a first polypeptide chain comprising an antibody heavy chain variable region sequence specific for a first target conjugated to a first polypeptide sequence comprising a first VpreB sequence, wherein the first polypeptide sequence comprising the first VpreB sequence is C-terminally conjugated to a second polypeptide sequence comprising a dimerization domain. In another embodiment, the first polypeptide chain is associated with a second polypeptide chain comprising a first polypeptide sequence that comprises a second VpreB sequence, wherein the first polypeptide sequence comprising the second VpreB sequence is C-terminally conjugated to an antibody heavy chain variable region sequence specific for a second target. In one other embodiment, the antibody heavy chain variable region sequence specific for a second target comprises a dimerization domain. In another embodiment, the dimerization domain comprises an antibody constant domain. In some embodiments, the dimerization domain comprises an Fc region. In another embodiment, the dimerization domain further comprises a protuberance or cavity. In another embodiment, one or both of the dimerization domains comprise an engineered amino acid sequence that promotes interaction between the dimerzation domains. In one embodiment, the engineered amino acid sequence comprises a region selected from the group consisting of: a complementary hydrophobic region, a complementary hydrophilic region, and a compatible protein-protein interaction domain. In another embodiment, the antibody heavy chain variable region sequence of the first polypeptide chain and the second VpreB sequence of the second polypeptide chain is capable of forming a binding site for said first polypeptide target.

In one other embodiment, the present invention provides a heteromeric bispecific binding protein comprising the first and second polypeptide chains, associated with each other. In one embodiment, the heavy chain variable region sequence specific for said second target of the second polypeptide and the first VpreB sequence of the first polypeptide chain form a binding site for a second target.

In another embodiment, the conjugation in the polypeptides or binding protein is by a linker sequence. In one embodiment, the linker sequence is heterologous linker sequence. In another embodiment, the conjugation is by direct fusion. In yet another embodiment, the linker sequence comprises a sequence selected from the group consisting of: an antibody J region sequence, a λ5 sequence, aλ light chain constant region sequence, a κ light chain constant region sequence, synthetic sequence, and any combination thereof.

In one embodiment, the VpreB sequence of the polypeptide chains or the heteromeric bispecific binding proteins is fused, at its C-terminus, to a heterologous sequence. In another embodiment, the heterogenous sequence is selected from the group consisting of a λ5 sequence and a light chain constant domain region sequence.

In another aspect, the multispecific SVD binding proteins may be provided in a bispecific monomeric stacked variable domain Surrobody structure format where the C-terminus of the dimerization domain is utilized for conjugation. In one embodiment, the present invention provides a first polypeptide chain comprising an antibody heavy chain variable region sequence specific for a first target C terminally conjugated to a first polypeptide sequence comprising a first VpreB sequence, wherein the N-terminus of the antibody heavy chain variable region sequence specific for a first target is conjugated to a dimerization domain. In another embodiment, the first polypeptide chain is associated with a second polypeptide chain comprising a first polypeptide sequence that comprises a second VpreB sequence, wherein the C-teiminus of the first polypeptide sequence comprising the second VpreB sequence is conjugated to an antibody heavy chain variable region sequence specific for a second target and the N-terminus of the first polypeptide sequence comprising the second VpreB sequence is conjugated to a dimerization domain. In one other embodiment, the dimerization domain comprises an antibody constant domain. In other embodiments, the dimerization domain comprises an Fc region. In another embodiment, the dimerization domain further comprises a protuberance or cavity. In another embodiment, one or both of the dimerization domains comprise an engineered amino acid sequence that promotes interaction between the dimerzation domains. In one embodiment, the engineered amino acid sequence comprises a region selected from the group consisting of: a complementary hydrophobic region, a complementary hydrophilic region, and a compatible protein-protein interaction domain. In another embodiment, the antibody heavy chain variable region sequence of the first polypeptide chain and the second VpreB sequence of the second polypeptide chain form a binding site for said first target.

In one other embodiment, the present invention provides a heteromeric bispecific binding protein comprising the first and second polypeptide chains, associated with each other. In one embodiment, the heavy chain variable region sequence specific for said second target of the second polypeptide and the first VpreB sequence of the first polypeptide chain form a binding site for a second target.

In another embodiment, the conjugation in the polypeptides or binding protein is by a linker sequence. In one embodiment, the linker sequence is heterologous linker sequence. In another embodiment, the conjugation is by direct fusion. In yet another embodiment, the linker sequence comprises a sequence selected from the group consisting of: an antibody J region sequence, a λ5 sequence, a λ light chain constant region sequence, a κ light chain constant region sequence, synthetic sequence, and any combination thereof.

In one embodiment, the VpreB sequence of the polypeptide chains or the heteromeric bispecific binding proteins is fused, at its C-terminus, to a heterologous sequence. In another embodiment, the heterogenous sequence is selected from the group consisting of a λ5 sequence, an antibody J-region sequence, and a light chain constant domain region sequence.

### E. Trispecific Stacked Variable Domain Surrobody Structures

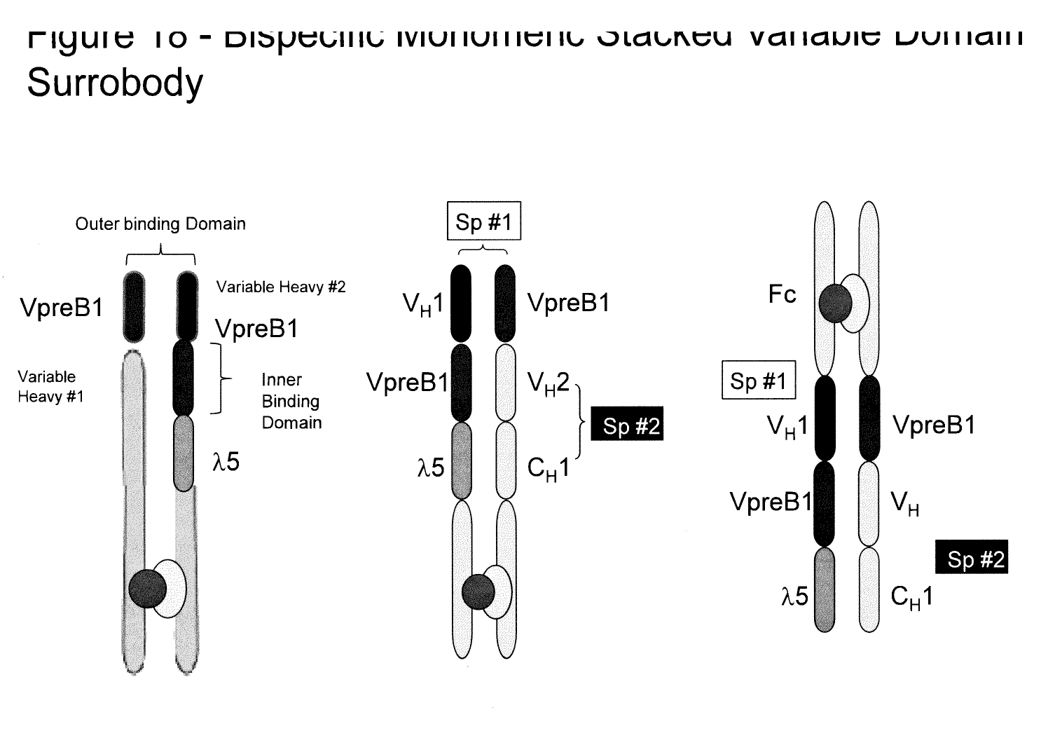

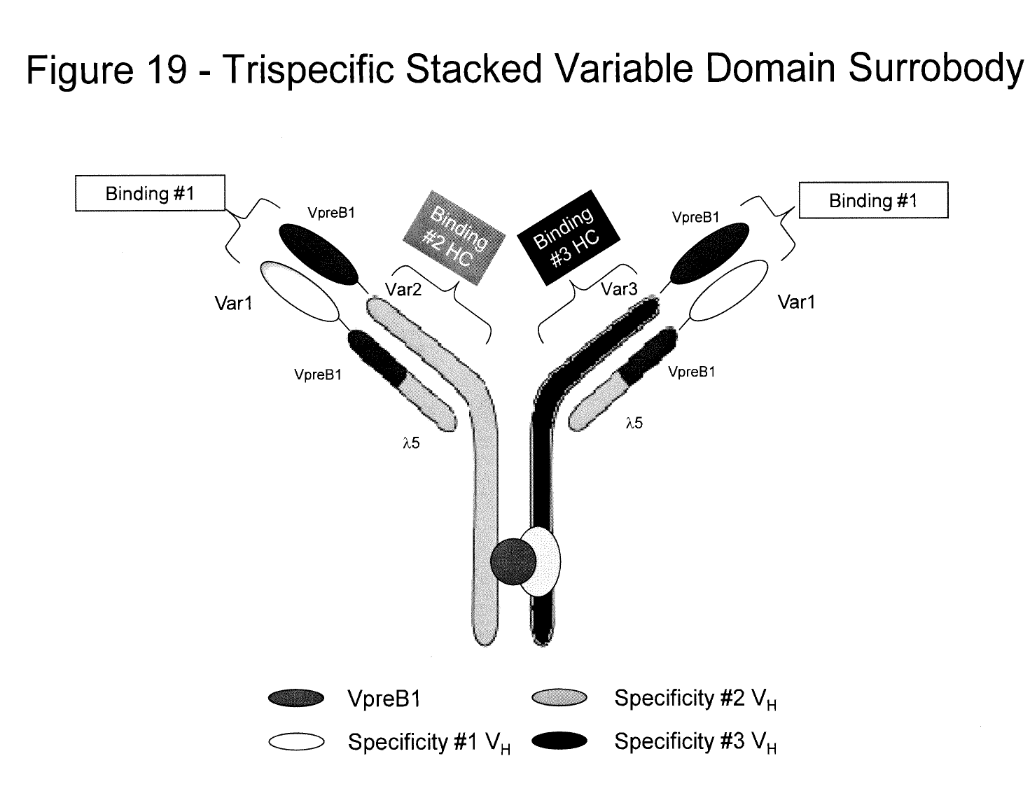

In another aspect, the multispecific SVD binding proteins may be provided in a trispecific stacked variable domain Surrobody structure format, as exemplified in FIG. 19. This format contemplates polypeptide chains and heteromultimeric bispecific binding proteins. The polypeptide chains are made up of antibody heavy chain variable (HCV) region sequences, two or more surrogate light chain (SLC) sequences, and dimerization domains. In one embodiment, the present invention provides a heteromeric trispecific binding protein comprising a first polypeptide chain comprising an antibody heavy chain variable region sequence specific for a first target, C-terminally conjugated to a polypeptide sequence comprising a first VpreB sequence, wherein the first polypeptide chain is associated with a) a second polypeptide chain comprising a polypeptide sequence that comprises a second VpreB sequence conjugated to the N-terminus of an antibody heavy chain comprising a variable region sequence specific for a second target; and b) a third polypeptide chain comprising a polypeptide sequence that comprises a third VpreB sequence conjugated to the N-terminus of an antibody heavy chain comprising a variable region sequence specific for a third target. In one embodiment, the antibody heavy chain variable region sequence specific for a second target comprises a dimerization domain. In another embodiment, the antibody heavy chain variable region sequence specific for a third target comprises a dimerization domain. In another embodiment, the dimerization domain comprises an antibody constant domain. In some embodiments, the dimerization domain comprises an Fc region. In another embodiment, the dimerization domain further comprises a protuberance or cavity. In another embodiment, one or both of the dimerization domains comprise an engineered amino acid sequence that promotes interaction between the dimerzation domains. In one embodiment, the engineered amino acid sequence comprises a region selected from the group consisting of: a complementary hydrophobic region, a complementary hydrophilic region, and a compatible protein-protein interaction domain. In another embodiment, the antibody heavy chain variable region sequence of the first polypeptide chain and the second VpreB sequence of the second polypeptide chain is capable of forming a binding site for said first polypeptide target.

In one other embodiment, the antibody heavy chain variable region sequence specific for a first target and the VpreB sequence of the second polypeptide chain form a binding site for said first target. In another embodiment, the antibody heavy chain variable region sequence specific for a first target and the VpreB sequence of the third polypeptide chain form a binding site for said first target. In one embodiment, the antibody heavy chain variable region sequence specific for a second target and the VpreB sequence of the first polypeptide chain form a binding site for said second target. In some embodiments, the antibody heavy chain variable region sequence specific for a third target and the VpreB sequence of the first polypeptide chain form a binding site for said third target.

In one embodiment, the association of the polypeptides of the heteromeric trispecific binding protein is covalent or non-covalent.

In another embodiment, the conjugation in the polypeptides or binding protein is by a linker sequence. In one embodiment, the linker sequence is heterologous linker sequence. In another embodiment, the conjugation is by direct fusion. In yet another embodiment, the linker sequence comprises a sequence selected from the group consisting of: an antibody J region sequence, a λ5 sequence, a λ light chain constant region sequence, a κ light chain constant region sequence, synthetic sequence, and any combination thereof.

In one embodiment, the VpreB sequence of the polypeptide chains or the heteromeric bispecific binding proteins is fused, at its C-terminus, to a heterologous sequence. In another embodiment, the heterogenous sequence is selected from the group consisting of a λ5 sequence and a light chain constant domain region sequence.

### F. “Cross-Complement” Stacked Variable Domain Surrobody Structures

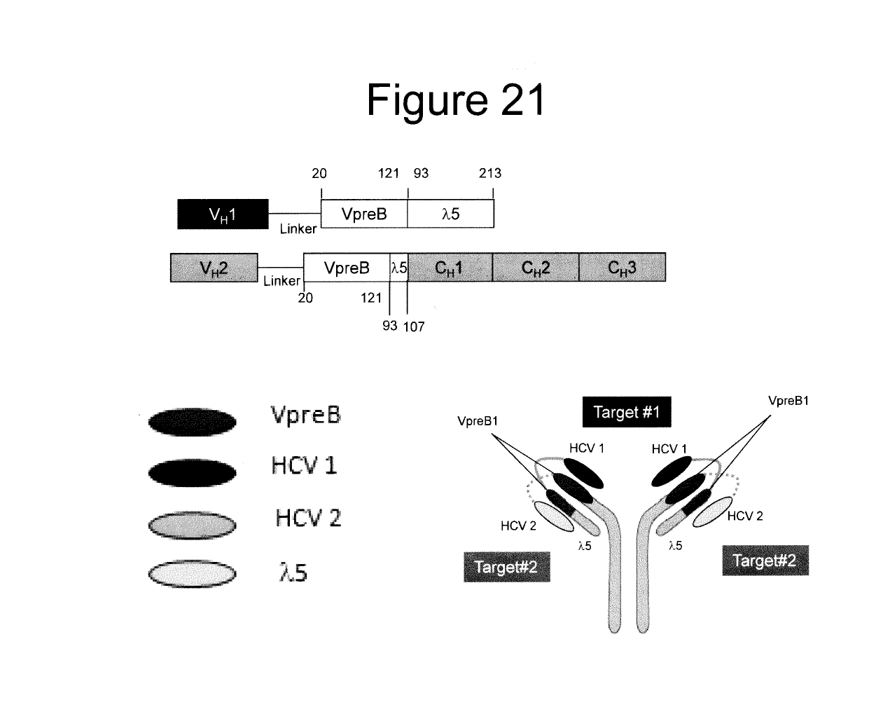

In another aspect, the multi-specific SVD binding proteins may be provided in a “cross-complemented” configuration as illustrated in FIG. 21. In one embodiment, a first polypeptide chain is provided comprising an antibody HC variable region sequence specific for a first target, C-terminally conjugated to a first polypeptide sequence comprising a first VpreB sequence, conjugated to a heterologous sequence. In another embodiment, the first polypeptide chain is associated with a second polypeptide chain comprising an antibody heavy chain variable region sequence specific for a second polypeptide target, C-terminally conjugated to a first polypeptide sequence comprising a second VpreB sequence. In one other embodiment, the first polypeptide sequence of the second polypeptide chain comprising the second VpreB sequence further comprises an antibody HC constant domain region. In one embodiment, the N-terminus of the antibody HC constant domain sequence is conjugated to the C-terminus of the first polypeptide sequence comprising the second VpreB sequence. In some embodiments, the antibody HC constant domain sequence comprises a CH 1 sequence and/or an Fc region. In another embodiment, the present invention provides a heteromeric bispecific binding protein comprising two pairs of the first and second polypeptides, associated with each other. In the heteromeric bispecific binding protein, a binding site for the first target is formed between the antibody HC variable domain region of the first polypeptide chain and the second VpreB sequence of the second polypeptide chain (e.g., Target#1 in FIG. 21). A binding site for the second target is formed between the antibody HC variable domain region of the second polypeptide chain and the first VpreB sequence of the first polypeptide chain (e.g., Target#2 in FIG. 21).

F. Stacked Variable Domain Surrobody Structure Formulas

The multispecific Surrobody molecules of the present invention may include at least four polypeptides. In one embodiment, the molecule has a) a first and second polypeptide having a sequence with the formula VH₁-X₁-SD₁, wherein VH₁ is a antibody heavy chain variable domain, X₁ is a linker, and SD₁ is a surrogate light chain domain; and b) a third and fourth polypeptide having a sequence with the formula SD₂-X₂-VH₂, wherein SD, is a surrogate light chain domain, X₂ is a linker, and VH, is a heavy chain variable domain (e.g., FIG. 1A). In one embodiment, the VH₂ may further include a sequence with the formula X₃-D, wherein X₃ is a linker and D is a dimerization domain. In another embodiment, the molecule is capable of binding to more than one target. The multispecific Surrobody may have an alternative format: a) a first and second polypeptide having a sequence with the formula VH₁-X₁-SD₁-X₂-SD₂, wherein VH₁ is an antibody heavy chain variable domain, X₁ is a linker, SD₁ is a surrogate light chain domain, X₂ is a linker, and SD₂ is a surrogate light chain domain; b) a third and fourth polypeptide having a VH₂, wherein VH, is an antibody HCV domain (e.g., FIG. 1B). In one embodiment, the VH₂ may further include a sequence with the formula X₁-D, wherein X₃ is a linker and D is a dimerization domain. The X₃ linker may be a peptide linker, or alternatively it may be omitted.

In an additional embodiment, the multispecific Surrobody may have a format corresponding to a) a first and second polypeptide having a sequence with the formula SD₁-X₁-SD₂ VH₁, wherein SD₁ is a surrogate light chain domain, X₁ is a linker, SD₂ is a surrogate light chain domain, and VH₁ is an antibody heavy chain variable domain; and b) a third and fourth polypeptide having a VH₂, wherein VH₂ is an antibody HCV domain (e.g., FIG. 1C). In one other embodiment, the molecule is capable of binding to more than one target.

In one other embodiment, the multispecific Surrobody may have a format corresponding to a) a first and second polypeptide having a sequence with the formula VH₁-X₁-SD₁-X₂-VH₂, wherein VH₁ is an antibody heavy chain variable domain, X₁ is a linker, SD₁ is a surrogate light chain domain, X₂ is a linker, and VH₂ is an antibody heavy chain variable domain; and b) a third and fourth polypeptide having a SD₂, wherein SD₂ is a surrogate light chain domain (e.g., FIG. 1D). In one embodiment, VH₂ further comprises an antibody heavy chain constant domain sequence. In one other embodiment, the molecule is capable of binding to more than one target.

In another embodiment, the multispecific Surrobody may have a format corresponding to a) a first and second polypeptide having a sequence with the formula VH₂-X₁-SD₁-X₂-VH₁, wherein VH₂ is an antibody heavy chain variable domain, X₁ is a linker, SD₁ is a surrogate light chain domain, X₂ is a linker, and VH₁ is an antibody heavy chain variable domain; and b) a third and fourth polypeptide having a SD₂, wherein SD₂ is a surrogate light chain domain (e.g., FIG. 1E). In one embodiment, VH, further comprises an antibody heavy chain constant domain sequence. In one other embodiment, the molecule is capable of binding to more than one target.

In an additional embodiment, the multispecific Surrobody may have a format corresponding to a) a first and second polypeptide having a sequence with the formula VH₂-X₁-SD₁-X₂-VH₁, wherein VH₂ is an antibody heavy chain variable domain, X₁ is a linker, SD₁ is a surrogate light chain domain, X₂ is a linker, and VH₁ is an antibody heavy chain variable domain; and b) a third and fourth polypeptide having a SD₂, wherein SD₂ is a surrogate light chain domain (e.g., FIG. 1E). In one embodiment, VH₂ further comprises an antibody heavy chain constant domain sequence. In one other embodiment, the molecule is capable of binding to more than one target.

In one other embodiment, the multispecific Surrobody may have a monomeric format corresponding to a polypeptide chain having a sequence with the formula VH₁-X₁-SD₁-X₂-VH₂-X₃-SD₂, wherein VH₁ is an antibody heavy chain variable domain, X₁ is a linker, SD₁ is a surrogate light chain domain, X₂ is a linker, VH₂ is an antibody heavy chain variable domain, X₃ is a linker, and SD, is a surrogate light chain domain. (e.g., FIG. 17). In one other embodiment, the molecule is capable of binding to more than one target.

In one other embodiment, the multispecific Surrobody may have a bivalent format corresponding to a first and second polypeptide chain having a sequence with the formula VH₁-X₁-SD₁-X₂-VH₂-X₃-SD₂-D, wherein VH₁ is an antibody heavy chain variable domain, X₁ is a linker, SD₁ is a surrogate light chain domain, X₂ is a linker, VH₂ is an antibody heavy chain variable domain, X₃ is a linker, SD₂ is a surrogate light chain domain, and D is a dimerization domain. (e.g., FIG. 17). In one other embodiment, the molecule is capable of binding to more than one target.

In one embodiment, the multispecific Surrobody may have a bispecific monomeric format (e.g., FIG. 18) corresponding to: a) a first polypeptide having a sequence with the formula SD₁-X₁-VH₁-D, wherein SD₁ is a surrogate light chain domain, X₁ is a linker, VH, is an antibody heavy chain variable domain, and D is a dimerization domain; and b) a second polypeptide having a sequence with the formula VH₂-X₁-SD₂-D, wherein VH₂ is an antibody heavy chain variable domain, X₁ is a linker, and SD₂ is a surrogate light chain domain. In one embodiment, D is an antibody heavy chain constant domain. In one other embodiment, the molecule is capable of binding to more than one target. In one embodiment, the multispecific Surrobody may have a bispecific monomeric format (e.g.,

FIG. 18) corresponding to: a) a first polypeptide having a sequence with the formula VH₁-X₁-SD₁-D, wherein VH₁ is an antibody heavy chain variable domain, X₁ is a linker, SD₁ is a surrogate light chain domain, and D is a dimerization domain; and b) a second polypeptide having a sequence with the formula SD₂-X₂-VH₂-D, wherein SD₂ is a surrogate light chain domain, X₂ is a linker, VH₂ is an antibody heavy chain variable domain, and D is a dimerization domain. In one embodiment, D is an antibody heavy chain constant domain. In one other embodiment, the molecule is capable of binding to more than one target.

In one embodiment, the multispecific Surrobody may have a bispecific monomeric format (e.g., FIG. 18) corresponding to: a) a first polypeptide having a sequence with the formula D-VH₁-X₁-SD₁, wherein D is a dimerization domain, VH, is an antibody heavy chain variable domain, X₁ is a linker, and SD₁ is a surrogate light chain domain; and b) a second polypeptide having a sequence with the formula D-SD₂-X₂-VH₂-CH1 wherein D is a dimerization domain, SD₂ is a surrogate light chain domain, X₂ is a linker, VH₂ is an antibody heavy chain variable domain, and CH1 is an antibody heavy chain constant CH1 region. In one embodiment, D is an antibody heavy chain constant domain. In one other embodiment, the molecule is capable of binding to more than one target.

In one embodiment, the multispecific Surrobody may have a tri-specific monomeric format (e.g., FIG. 19) corresponding to: a) a first and second polypeptide having a sequence with the formula VH₁-X₁-SD₁, wherein VH₁ is an antibody heavy chain variable domain, X₁ is a linker, and SD₁ is a surrogate light chain domain; and b) a second polypeptide having a sequence with the formula SD₂-X₂-VH₂-D, wherein SD₂ is a surrogate light chain domain, X₂ is a linker, VH₂ is an antibody heavy chain variable domain, and D is a dimerization domain; c) a third polypeptide having a sequence with the formula SD₃-X₃-VH₃-D, wherein SD₃ is a surrogate light chain domain, X₃ is a linker, VH₃ is an antibody heavy chain variable domain, and D is a dimerization domain. In one embodiment, D is an antibody heavy chain constant domain. In one other embodiment, the molecule is capable of binding to more than two targets.

In another embodiment, the multispecific Surrobody may have a cross-complement format (e.g., FIG. 21) corresponding to: a) a first and second polypeptide having a sequence with the formula VH₁-X₁-SD₁, wherein VH₁ is an antibody heavy chain variable domain, X₁ is a linker, and SD₁ is a surrogate light chain domain; b) a third and fourth polypeptide having a sequence with the formula VH₂-X₂-SD₂-CH, wherein VH, is an antibody heavy chain variable domain, SD₂ is a surrogate light chain domain, X₂ is a linker, and CH is an antibody heavy chain constant domain. In one other embodiment, the molecule is capable of binding to more than one target.

For multispecific Surrobodies, the Surrobody light chain (SLC) domain may include one or more SLC polypeptides. In one embodiment, the SLC domain is an SLC polypeptide conjugated to a heterologous amino acid sequence. The heterologous amino acid sequence may be another SLC polypeptide. For example, a VpreB polypeptide may be conjugated to a λ5 polypeptide, or a Vκ-like polypeptide may be conjugated to a JCκ polypeptide. In one embodiment, the conjugate may be a fusion. Examples of multispecific Surrobody molecules are depicted in FIGS. 1A-F, 17-19, and 21.

### G. Surrogate Light Chain Domains

The multispecific Surrobody molecules described herein comprise surrogate light chain (SLC) domains and have the ability to bind more than one target. The targets can be any peptide or polypeptide that is a binding partner for the SLC polypeptides of the present invention. Targets specifically include all types of targets generally referred to as “antigens” in the context of antibody binding.

The surrogate light chain (SLC) constructs herein are based on the pre-B cell receptor (pre-BCR), which is produced during normal development of an antibody repertoire. Unlike antibodies, pre-BCR is a trimer, that is composed of an antibody heavy chain paired with two surrogate light chain components, VpreB and λ5. Both VpreB and λ5 are encoded by genes that do not undergo gene rearrangement and are expressed in early pro-B cells before V(D)J recombination begins. The pre-BCR is structurally different from a mature immunoglobulin in that it is composed of a heavy chain and two non-covalently associated proteins: VpreB and λ5, i.e., they have three components as opposed to two in antibodies. Furthermore, although VpreB is homologous to the Vλ Ig domain, and λ5 is homologous to the Cλ domain of antibodies, each has noncanonical peptide extensions: VpreB1 has additional 21 residues on its C terminus; λ5 has a 50 amino acid extension at its N terminus.

Similarly, the κ-like surrogate light chain constructs described herein are based on the pre-B cell receptor (pre-BCR). The κ-like light chain is the germline VκIV gene partnered with a JCκ fusion gene. In each of these genes a peptidic extension exists in the vicinity surrounding a site analogous for CDR3. As these two proteins do not appear to recombine at the genomic level it is likely their association to a heavy chain are mutually exclusive of each other and analogous to the associations described for the λ-like surrogate light chain.

Further details of the design and production of Surrobodies are provided in Xu et al., Proc. Natl. Acad. Sci. USA 2008, 105(31):10756-61 and in PCT Publications WO 2008/118970, published on Oct. 2, 2008; WO/2010/006286, published on Jan. 1, 2010; and WO/2010/151808, published on Dec. 29, 2010 (the contents of which are each incorporated herein by reference in their entirety).

(i) λ-Like Surrogate Light Chains

The present invention contemplates multispecific Surrobody molecules comprising SLC domains that have a VpreB sequence conjugated to a λ5 sequence. In one embodiment, the VpreB sequence is selected from the group consisting of a native VpreB1 sequence, a native VpreB2 sequence, a native VpreB3 sequence and fragments and variants thereof. In one other embodiment, the native VpreB sequence is selected from the group consisting of human VpreB 1 of SEQ ID NO: 1, mouse VpreB2 of SEQ ID NOS: 2 and 3, human VpreB3 of SEQ ID NO: 4, human VpreB-like polypeptide of SEQ ID NO:5, human VpreB dTail polypeptide of SEQ ID NO:6 and fragments and variants thereof. In other embodiments, the λ5 sequence comprises all or part of a murine λ5-like of SEQ ID NO: 7; a human λ5 polypeptide of SEQ ID NO: 8, a human λ5 dTail polypeptide of SEQ ID NO:9, or the human λ5 dTail sequence with a murine Ig κ leader sequence (SEQ ID NO: 10).

The main isoform of human VpreB1 (CAG30495) is a 145 amino acid long polypeptide (SEQ ID NO: 1 in FIG. 5), including a 19 amino acid leader sequence. Similar leader sequences are present in other VpreB polypeptides. The human truncated VpreB1 sequence (lacking the characteristic “tail” at the C-terminus of native VpreB1), is also referred to as the “VpreB1 dTail sequence” and shown as SEQ ID NO:5.

The main isoform of murine λ5 (CAA10962) is a 209-amino acid polypeptide (SEQ ID NO:7), including a 30 amino acid leader sequence. A human λ5-like protein has 213 amino acids (NP_{—}064455; SEQ ID NO: 8) and shows about 84% sequence identity to the antibody λ light chain constant region. Similar leader sequences are present in other λ5 polypeptides. The human truncated λ5 sequence (lacking the characteristic “tail” at the N-terminus of native λ5), is also referred to as the “λ5 dTail sequence” and shown as SEQ ID NO:9.

In one other embodiment, the invention provides an SLC construct comprising a VpreB sequence shown as SEQ ID NO:6. In another embodiment, the invention provides an SLC construct comprising a λ5 sequence shown as SEQ ID NO:10. In one embodiment, the invention provides an SLC construct comprising a polypeptide shown as SEQ ID NO:35.

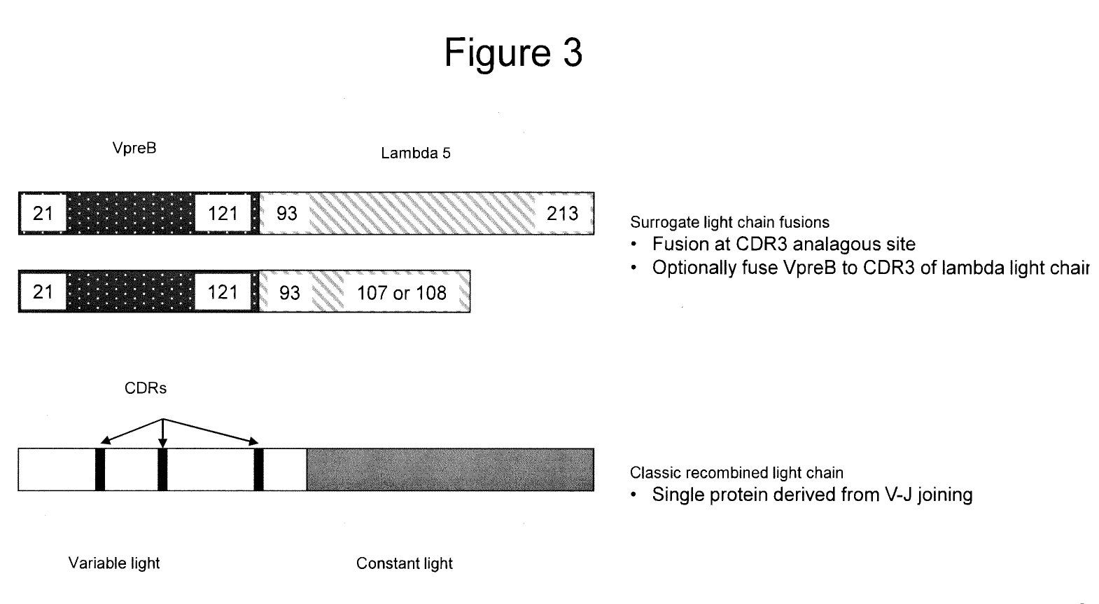

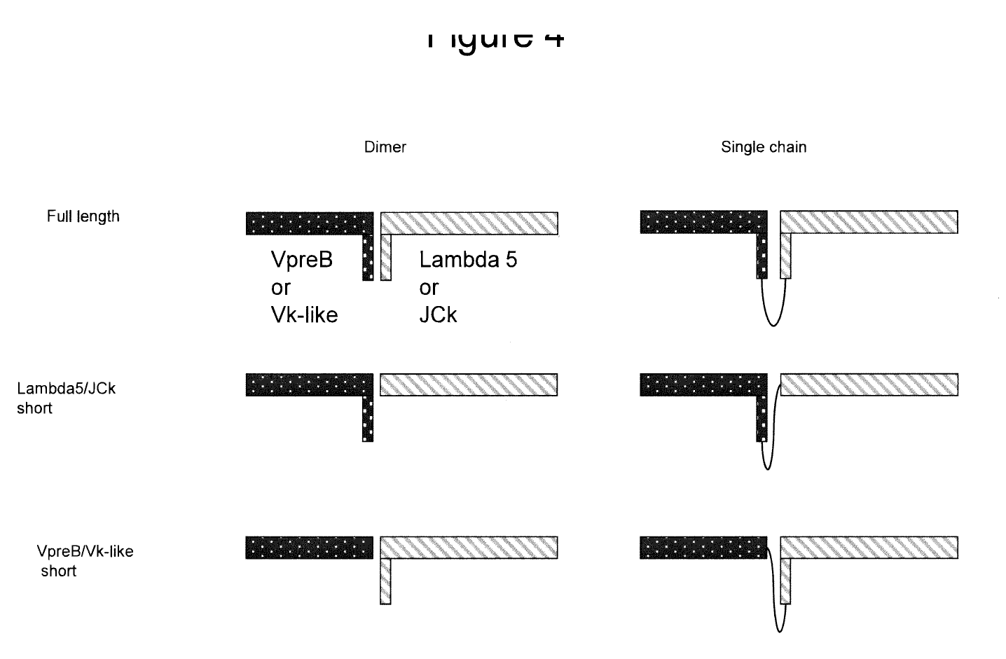

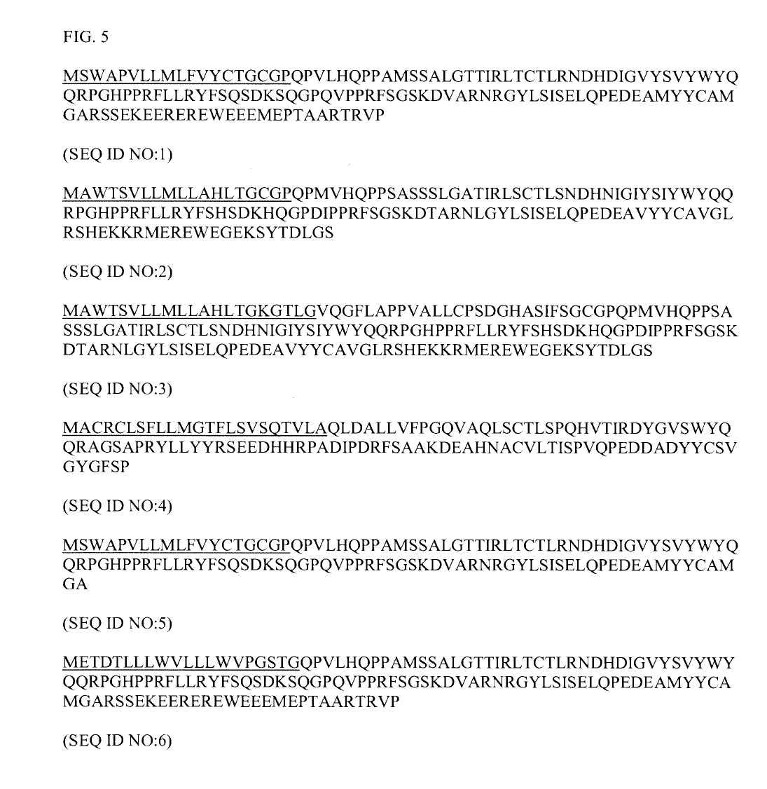

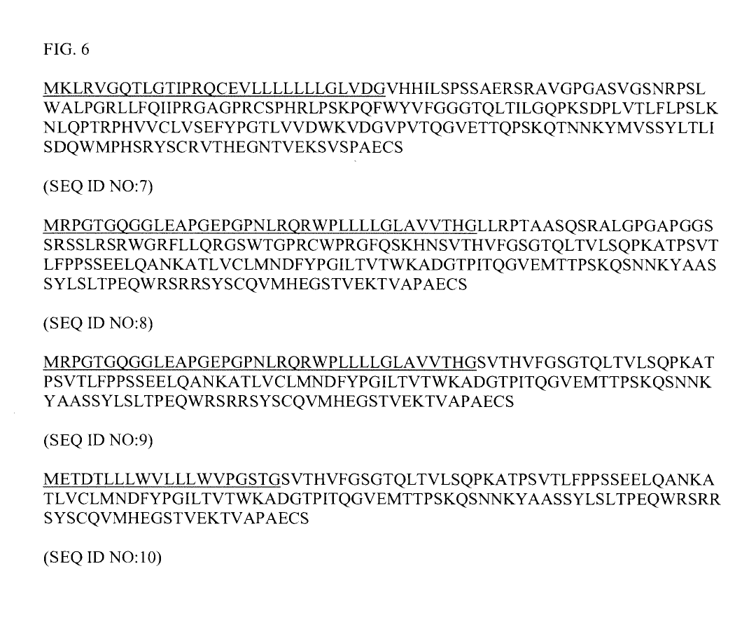

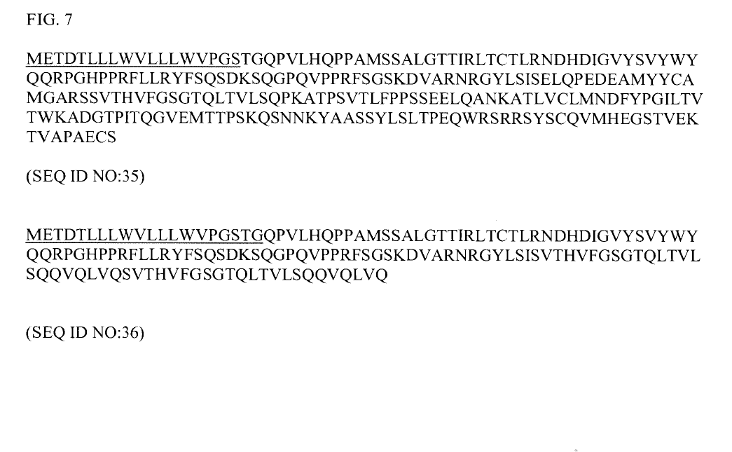

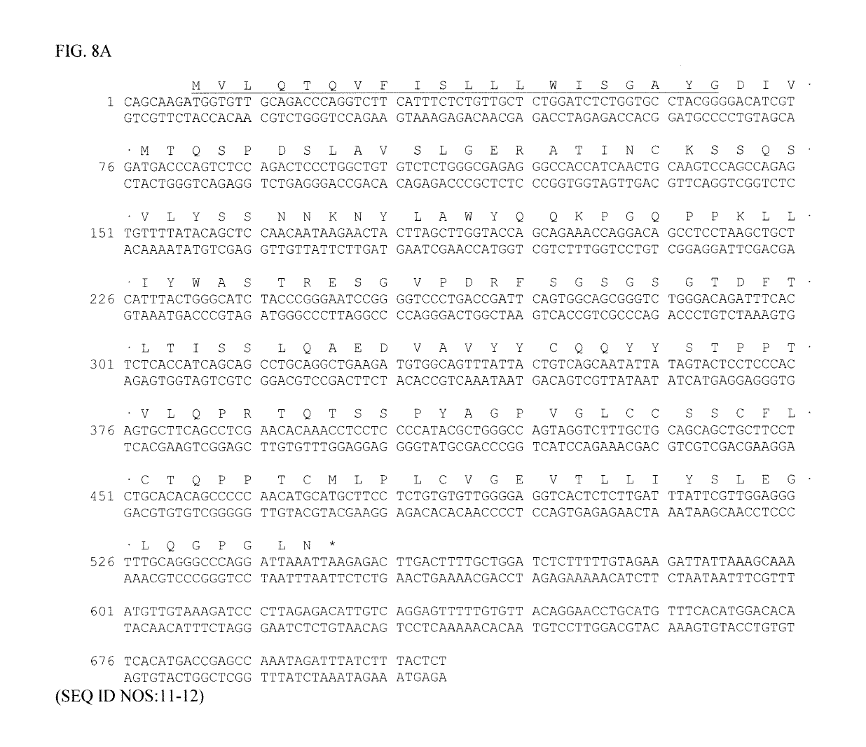

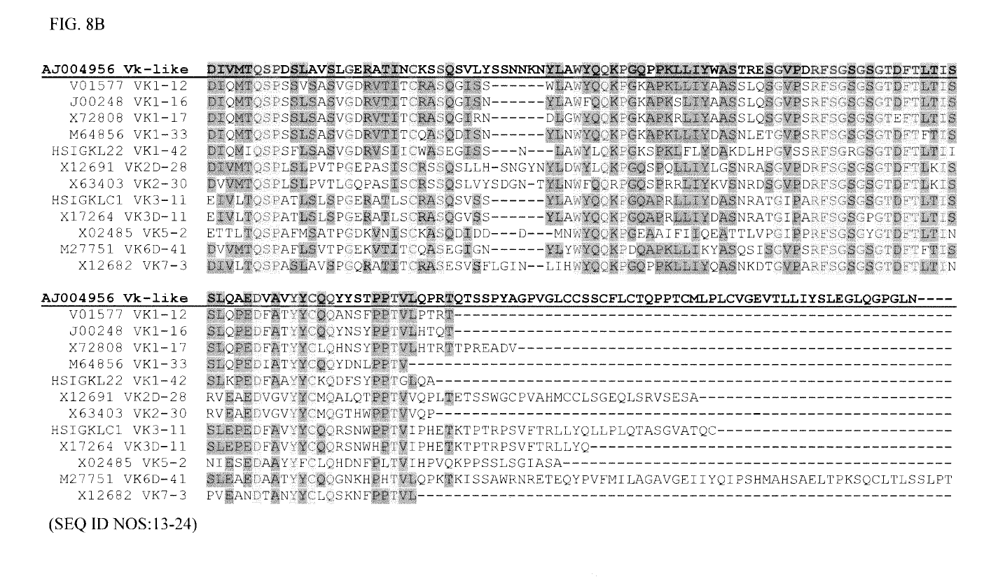

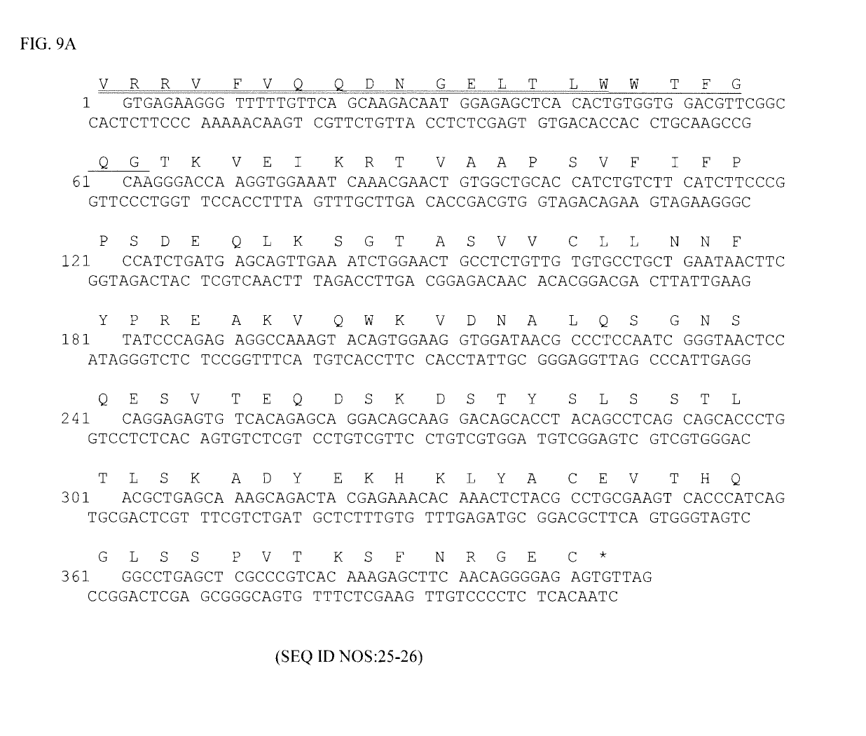

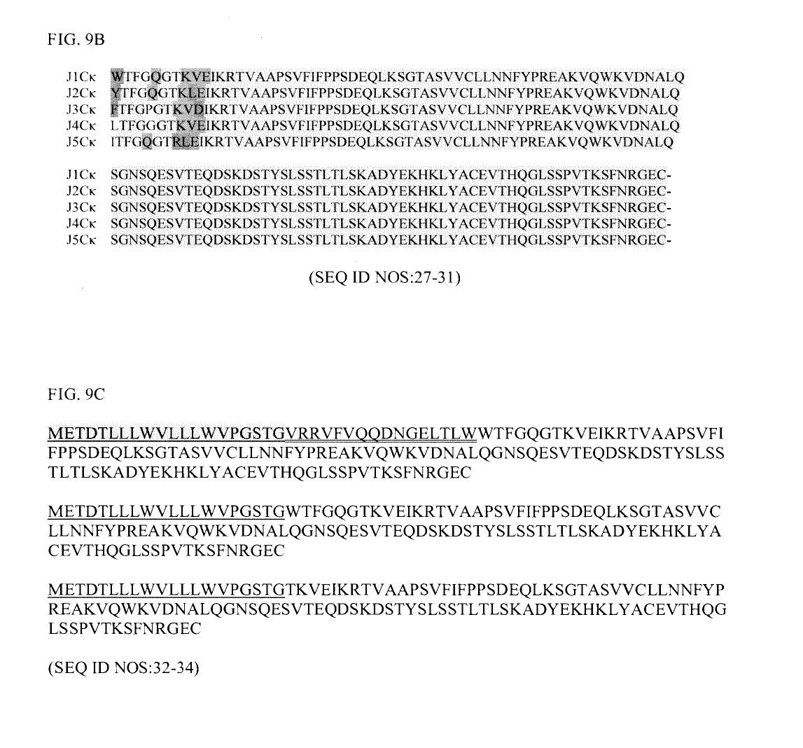

Specific examples of λ-like Surrobodies include polypeptides in which a VpreB sequence, such as a VpreB1, VpreB2, or VpreB3 sequence, including fragments and variants of the native sequences, is conjugated to a λ5 sequence, including fragments and variants of the native sequence. Representative fusions of this type are provided in PCT Publication WO 2008/118970 published on Oct. 2, 2008, the entire disclosure of which are expressly incorporated by reference herein. An example of a fusion with a heterologous leader sequence is illustrated in FIG. 7 (SEQ ID NOS: 35 and 36). In a direct fusion, typically the C-terminus of a VpreB sequence (e.g. a VpreB1, VpreB2 or VpreB3 sequence) is fused to the N-terminus of a λ5 sequence. While it is possible to fuse the entire length of a native VpreB sequence to a full-length λ5 sequence, typically the fusion takes place at or around a CDR3 analogous site in each of the two polypeptides. A representative fusion construct based on the analogous CDR3 sites for VpreB1 and λ5 is illustrated in FIG. 3. In this embodiment, the fusion may take place within, or at a location within about 10 amino acid residues at either side of the CDR3 analogous region. In a preferred embodiment, the fusion takes place between about amino acid residues 116-126 of the native human VpreB1 sequence (SEQ ID NO: 1) and between about amino acid residues 82 and 93 of the native human λ5 sequence (SEQ ID NO: 8).

As noted above, in addition to direct fusions, the polypeptide constructs of the present invention include non-covalent associations of a VpreB sequence (including fragments and variants of a native sequence) with a heterologous sequence, such as a λ5 sequence (including fragments and variants of the native sequence), and/or an antibody sequence. Thus, for example, a full-length VpreB sequence may be non-covalently associated with a truncated λ5 sequence. Alternatively, a truncated VpreB sequence may be non-covalently associated with a full-length λ5 sequence.

Surrogate light chain constructs comprising non-covalently associated VpreB 1 and &5 sequences, in association with an antibody heavy chain. The association may be covalent and/or non-covalent. The structures may include, for example, full-length VpreB 1 and λ5 sequences, a full-length VpreB 1 sequence associated with a truncated λ5 sequence (“Lambda 5dT”), a truncated VpreB1 sequence associated with a full-length λ5 sequence (VpreB dT”) and a truncated VpreB 1 sequence associated with a truncated λ5 sequence (“Short”).

One of ordinary skill will appreciate that a variety of other constructs can be made and used in a similar fashion. For example, the structures can be asymmetrical, comprising different surrogate light chain sequences in each arm, and/or having trimeric or pentameric structures.

All surrogate light chain constructs (Surrobodies) herein may be associated with antibody sequences. For example, a polypeptide comprising one or more VpreB-λ5 fusions can be linked to an antibody heavy chain variable region sequence by a peptide linker. In another embodiment, a VpreB-λ5 fusion is non-covalently associated with an antibody heavy chain, or a fragment thereof including a variable region sequence to form a dimeric complex. In yet another embodiment, the VpreB and λ5 sequences are non-covalently associated with each other and an antibody heavy chain, or a fragment thereof including a variable region sequence, thereby footling a trimeric complex.

In one embodiment, the invention provides an SLC construct wherein the λ5 sequence is non-covalently associated with the VpreB sequence. In one other embodiment, the invention contemplates an SLC construct wherein the conjugate of said VpreB sequence and λ5 sequence is non-covalently associated with an antibody heavy chain sequence.

The present invention also contemplates SLC constructs wherein a λ5 sequence and a VpreB sequence are connected by a covalent linker. In one embodiment, the invention provides an SLC construct wherein the λ5 sequence is non-covalently associated with the VpreB sequence. In one other embodiment, the invention contemplates an SLC construct wherein the conjugate of said VpreB sequence and λ5 sequence is non-covalently associated with an antibody heavy chain sequence.

The multispecific Surrobody molecules of the present invention may contain VpreB/λ5 conjugates. The conjugates may be SLC polypeptides that are fusions. Exemplary sequences suitable for use in VpreB1 (SEQ ID NO: 1)/λ5 (SEQ ID NO: 8) conjugates include, without limitation, VpreB1(20-121), λ5 (93-213), λ5 (93-107), and λ5 (93-108).

In another aspect, the multispecific Surrobody molecules will have SLC polypeptides conjugated to an antibody heavy chain domain. In one embodiment, the conjugate is a fusion. The fusions may have particular linkers between the SLC polypeptides and the heavy chain polypeptide. Exemplary sequences suitable to link the SLC and the heavy chain include, without limitation, sequences comprising

Ala Ser,
Ala Ser Thr,

(SEQ ID NO: 112)

Ala Ser Thr Lys,

(SEQ ID NO: 113)

Ala Ser Thr Lys Gly,

(SEQ ID NO: 114)

Ala Ser Thr Lys Gly Pro,

(SEQ ID NO: 115)

Ala Ser Thr Lys Gly Pro Ser,

(SEQ ID NO: 116)

Ala Ser Thr Lys Gly Pro Ser Val,

(SEQ ID NO: 117)

Ala Ser Thr Lys Gly Pro Ser Val Phe,
and

(SEQ ID NO: 118)

Ala Ser Thr Lys Gly Pro Ser Val Phe Pro.

In one embodiment, the linking sequence links an antibody variable heavy chain domain with an SLC domain. The linking sequence may be a CH1 amino acid sequence. In another embodiment, the linking sequence is carboxy-terminal to the heavy chain variable domain and/or amino-terminal to the SLC domain. In one other embodiment, X₁ of the formula VH₁-X₁-SD₁ may comprise one of the CH1 amino acid sequences.

In one embodiment, the (G4S)3 linker sequence (Gly Gly Gly Gly Ser Gly Gly Gly Gly Ser Gly Gly Gly Gly Ser—SEQ ID NO: 119) or variant thereof, is used as a synthetic linker in any part of the multispecific stacked variable domain binding proteins. For example, the linker sequence is used alone or in combination with the linkers described above that link the antibody variable heavy chain domain with an SLC domain.

In another embodiment, the linker sequence is amino-terminal to the heavy chain variable domain and/or carboxy-terminal to the SLC domain. Exemplary sequences suitable to link the SLC and the heavy chain include, without limitation, sequences comprising

Ser Gln,
Ser Gln Pro,

(SEQ ID NO: 120)

Ser Gln Pro Lys,

(SEQ ID NO: 121)

Ser Gln Pro Lys Ala,

(SEQ ID NO: 122)

Ser Gln Pro Lys Ala Thr,

(SEQ ID NO: 123)

Ser Gln Pro Lys Ala Thr Pro,

(SEQ ID NO: 124)

Ser Gln Pro Lys Ala Thr Pro Ser,

(SEQ ID NO: 125)

Ser Gln Pro Lys Ala Thr Pro Ser Val,

(SEQ ID NO: 126)

Ser Gln Pro Lys Ala Thr Pro Ser Val Thr,
and

(SEQ ID NO: 127)

Ser Gln Pro Lys Ala Thr Pro Ser Val Thr Gly Gly
Gly Gly Ser.

In one embodiment, the linking sequence links an antibody variable heavy chain domain with an SLC domain. The SLC domain may comprise a λ5 amino acid sequence. In one other embodiment, X₂ of the formula SD₂-X₂—VH₂ may be one of the λ5 amino acid sequences.

In one other aspect, the multispecific Surrobody molecules will have amino acid junction or linkage regions comprising sequences from an antibody heavy chain variable (HCV) domain, an antibody heavy chain constant domain, and an SLC domain. In one embodiment, the HCV domain is amino-terminal to the SLC domain and separated by the heavy chain constant domain sequence (e.g., Ala Ser, Ala Ser Thr; etc. as shown below). Exemplary sequences suitable as linkage regions include, without limitation, sequences comprising

(SEQ ID NO: 67)

Xaa_{g} Ala Ser Xaaₕ,

(SEQ ID NO: 68)

Xaa_{g} Ala Ser Thr Xaaₕ,

(SEQ ID NO: 69)

Xaa_{g} Ala Ser Thr Lys Xaaₕ,

(SEQ ID NO: 70)

Xaa_{g} Ala Ser Thr Lys Gly Xaaₕ,

(SEQ ID NO: 71)

Xaa_{g} Ala Ser Thr Lys Gly Pro Xaaₕ,

(SEQ ID NO: 72)

Xaa_{g} Ala Ser Thr Lys Gly Pro Ser Xaaₕ,

(SEQ ID NO: 73)

Xaa_{g} Ala Ser Thr Lys Gly Pro Ser Val Xaaₕ,

(SEQ ID NO: 74)

Xaa_{g} Ala Ser Thr Lys Gly Pro Ser Val Phe Xaaₕ,
and

(SEQ ID NO: 75)

Xaa_{g} Ala Ser Thr Lys Gly Pro Ser Val Phe Pro Xaaₕ.

The underlined region is an antibody heavy chain CH1 sequence. Xaa is any amino acid, g is 1 to 10 amino acids, and h is 1 to 10 amino acids. Xaa_{g} may be an antibody heavy chain variable domain sequence and Xaa₁, may be a VpreB sequence. The formula VH₁-X₁-SD₁ or VH₁-X₁-SD₁-CH (corresponding to a cross-complemented SVD format discussed below) may comprise one of these linkage regions. Exemplary sequences suitable for Xaa_{g} include, without limitation, sequences comprising

Ser,
Ser Ser,
Val Ser Ser,

(SEQ ID NO: 76)

Thr Val Ser Ser,

(SEQ ID NO: 77)

Val Thr Val Ser Ser,

(SEQ ID NO: 78)

Leu Val Thr Val Ser Ser,

(SEQ ID NO: 79)

Thr Leu Val Thr Val Ser Ser,

(SEQ ID NO: 80)

Gly Thr Leu Val Thr Val Ser Ser,

(SEQ ID NO: 81)

Gln Gly Thr Leu Val Thr Val Ser Ser,
and

(SEQ ID NO: 82)

Gly Gln Gly Thr Leu Val Thr Val Ser Ser.

Exemplary sequences for Xaaₐ include, without limitation, sequences comprising

Gln,
Gln Pro,
Gln Pro Val,
Gln Pro Val Leu,

(SEQ ID NO: 83)

Gln Pro Val Leu His,

(SEQ ID NO: 84)

Gln Pro Val Leu His Gln,

(SEQ ID NO: 85)

Gln Pro Val Leu His Gln Pro,

(SEQ ID NO: 86)

Gln Pro Val Leu His Gln Pro Pro,

(SEQ ID NO: 87)

Gln Pro Val Leu His Gln Pro Pro Ala,
and

(SEQ ID NO: 88)

Gln Pro Val Leu His Gln Pro Pro Ala Met.

In another embodiment, the HCV domain is carboxy-terminal to the SLC domain and separated by a λ5 sequence. Exemplary sequences suitable as linkage regions include, without limitation, sequences comprising

(SEQ ID NO: 89)

Xaaⱼ Ser Gln Xaaₖ,

(SEQ ID NO: 90)

Xaaⱼ Ser Gln Pro Xaaₖ,

(SEQ ID NO: 91)

Xaaⱼ Ser Gln Pro Lys Xaaₖ,

(SEQ ID NO: 92)

Xaaⱼ Ser Gln Pro Lys Ala Xaaₖ,

(SEQ ID NO: 93)

Xaaⱼ Ser Gln Pro Lys Ala Thr Xaaₖ,

(SEQ ID NO: 94)

Xaaⱼ Ser Gln Pro Lys Ala Thr Pro Xaaₖ,

(SEQ ID NO: 95)

Xaaⱼ Ser Gln Pro Lys Ala Thr Pro Ser Xaaₖ,

(SEQ ID NO: 96)

Xaaⱼ Ser Gln Pro Lys Ala Thr Pro Ser Val Xaaₖ,

(SEQ ID NO: 97)

Xaaⱼ Ser Gln Pro Lys Ala Thr Pro Ser Val Thr Xaaₖ,
and

(SEQ ID NO: 98)

Xaaⱼ Ser Gln Pro Lys Ala Thr Pro Ser Va Thr Gly
Gly Gly Gly Ser Xaaₖ.

The underlined region is a λ5 sequence. Xaa is any amino acid, j is 1 to 10 amino acids, and k is 1 to 6 amino acids.

The formula SD₂-X₂-VH₂ may comprise one of these linkage regions. Xaa, may comprise a λ5 sequence. Exemplary sequences suitable for Xaa, include, without limitation, sequences comprising

Leu,
Val Leu,
Thr Val Leu,

(SEQ ID NO: 99)

Leu Thr Val Leu,

(SEQ ID NO: 100)

Gln Leu Thr Val Leu,

(SEQ ID NO: 101)

Thr Gln Leu Thr Val Leu,

(SEQ ID NO: 102)

Gly Thr Gln Leu Thr Val Leu,

(SEQ ID NO: 103)

Ser Gly Thr Gln Leu Thr Val Leu,
and

(SEQ ID NO: 104)

Gly Ser Gly Thr Gln Leu Thr Val Leu.

Xaaₖ may comprise an antibody heavy chain variable sequence. Exemplary sequences suitable for Xaaₖ include, without limitation, sequences comprising

Gln,
Gln Val,
Gln Val Gln,

(SEQ ID NO: 105)

Gln Val Gln Leu,

(SEQ ID NO: 106)

Gln Val Gln Leu Val,
and

(SEQ ID NO: 107)

Gln Val Gln Leu Val Gln.

In one embodiment, the Xaaₖ is a sequence from a heavy chain germline including, without limitation, V_{H}1 1-3 1-02, and V_{H}1 1-2 1-e. Other exemplary germline sequences suitable for Xaaₖ include, without limitation, Glu, Glu Val, Glu Val Gln, and Glu Val Gln Leu (SEQ ID NO: 105).

In one aspect, the present invention provides multispecific Surrobody molecules that include a single chain Surrobody fragment, also referred to as an scSv. The scSv may be an antibody heavy chain variable domain conjugated to a first SLC polypeptide having a first SLC domain. In one embodiment, the conjugate is a fusion. In another embodiment, the first SLC domain is a λ-like SLC domain. The fusions may have particular junctions or linkage regions between the first SLC polypeptide and the heavy chain polypeptide. In one embodiment, the linkage region comprises a (G4S)3 sequence (Gly Gly Gly Gly Ser Gly Gly Gly Gly Ser Gly Gly Gly Gly Ser—SEQ ID NO: 119). The (G4S)3 sequence may be located carboxy-terminal to the heavy chain variable domain and amino-terminal to the SLC domain. In such cases, suitable linkage regions include, without limitation, sequences comprising Xaa_{g} (Gly4Ser)3 Xaaₕ (SEQ ID NO: 12). Xaa_{g} may be an antibody heavy chain variable domain sequence. Xaa may comprise a λ5 sequence. The formula VH₁-X₁-SD₁-X₂-SD, may comprise one of these linkage regions.

Exemplary sequences suitable for Xaa_{g} include, without limitation, sequences comprising

Ser,
Ser Ser,
Val Ser Ser,

(SEQ ID NO: 76)

Thr Val Ser Ser,

(SEQ ID NO: 77)

Val Thr Val Ser Ser,

(SEQ ID NO: 78)

Leu Val Thr Val Ser Ser,

(SEQ ID NO: 79)

Thr Leu Val Thr Val Ser Ser,

(SEQ ID NO: 80)

Gly Thr Leu Val Thr Val Ser Ser,

(SEQ ID NO: 81)

Gln Gly Thr Leu Val Thr Val Ser Ser,
and

(SEQ ID NO: 82)

Gly Gln Gly Thr Leu Val Thr Val Ser Ser.

Xaaₕ may be a VpreB sequence. Exemplary sequences for Xaaₕ include, without limitation, sequences comprising

Gln,
Gln Pro,
Gln Pro Val,

(SEQ ID NO:

Gln Pro Val Leu,

(SEQ ID NO: 83)

Gln Pro Val Leu His,

(SEQ ID NO: 84)

Gln Pro Val Leu His Gln,

(SEQ ID NO: 85)

Gln Pro Val Leu His Gln Pro,

(SEQ ID NO: 86)

Gln Pro Val Leu His Gln Pro Pro,

(SEQ ID NO: 87)

Gln Pro Val Leu His Gln Pro Pro Ala,
and

(SEQ ID NO: 88)

Gln Pro Val Leu His Gln Pro Pro Ala Met.

The scSv molecules may also have a second SLC polypeptide with a second SLC domain conjugated to the first SLC polypeptide. In one embodiment, the conjugate is a fusion. In another embodiment, the second SLC domain is a λ-like SLC domain. The second SLC polypeptide may be located carboxy-terminal to the first SLC polypeptide. The fusions may have particular junctions or linkage regions between the first and the second SLC polypeptides. In one embodiment, the linking sequence contains a Gly Ala (GA) sequence. The GA sequence may be located carboxy-terminal to the first SLC polypeptide and amino-terminal to the second SLC polypeptide. In such cases, suitable linkage regions include, without limitation, sequences comprising Xaaₙ Gly Ala Xaaₕ (SEQ ID NO: 129). Xaaₙ is a first SLC domain sequence. Xaa is any amino acid, n is 1 to 10 amino acids, and h is 1 to 10 amino acids. Xaaₙ may be a λ5 sequence. The formula VH₁-X₁-SD₁-X₂-SD, may comprise one of these linkage regions.

Exemplary sequences suitable for Xaaₙ include, without limitation, sequences comprising

Ser,
Leu Ser,
Val Leu Ser,

(SEQ ID NO: 130)

Thr Val Leu Ser,,

(SEQ ID NO: 131)

Leu Thr Val Leu Ser,

(SEQ ID NO: 132)

Gln Leu Thr Val Leu Ser,

(SEQ ID NO: 133)

Thr Gln Leu Thr Val Leu Ser,

(SEQ ID NO: 134)

Gly Thr Gln Leu Thr Val Leu Ser,

(SEQ ID NO: 135)

Ser Gly Thr Gln Leu Thr Val Leu Ser,
and

(SEQ ID NO: 136)

Gly Ser Gly Thr Gln Leu Thr Val Leu Ser.

Xaaₕ may be a VpreB sequence. Exemplary sequences for Xaaₕ include, without limitation, sequences comprising

Gln,
Gln Pro,
Gln Pro Val,

(SEQ ID NO: 128)

Gln Pro Val Leu,,

(SEQ ID NO: 83)

Gln Pro Val Leu His,

(SEQ ID NO: 84)

Gln Pro Val Leu His Gln,

(SEQ ID NO: 85)

Gln Pro Val Leu His Gln Pro,

(SEQ ID NO: 86)

Gln Pro Val Leu His Gln Pro Pro,

(SEQ ID NO: 87)

Gln Pro Val Leu His Gln Pro Pro Ala,
and

(SEQ ID NO: 88)

Gln Pro Val Leu His Gln Pro Pro Ala Met.

In another aspect, the multispecific Surrobody molecule is a cross-complemented SVD molecule (e.g., FIG. 21) having a first amino acid junction or linkage region comprising sequences from an antibody heavy chain variable (HCV) domain, an antibody heavy chain constant domain, and an SLC domain, as described above. In one other embodiment, the cross-complemented SVD molecule further comprises a second or additional amino acid junction or linkage region comprising sequences from an SLC sequence and an antibody heavy chain constant domain region. The formula VH₁-X₁-SD₁-CH may comprise a first and a second linkage regions. Suitable sequences for the second or additional linkage regions include, without limitation, sequences comprising Xaaₛ Ser Xaaᵣ (SEQ ID NO: 144), wherein Xaa is any amino acid, s is 1 to 10 amino acids, and r is 1 to 10 amino acids. In one embodiment, Xaaₛ comprises a VpreB sequence and/or a sequence. In another embodiment, Xaa, comprises an antibody heavy chain constant domain sequence. Exemplary sequences suitable for Xaaₛ include, without limitation, sequences comprising

Gly,
Gly Ser,
Gly Ser Gly,

(SEQ ID NO: 137)

Gly Ser Gly Thr,

(SEQ ID NO: 138)

Gly Ser Gly Thr Gln,

(SEQ ID NO: 139)

Gly Ser Gly Thr Gln Leu,

(SEQ ID NO: 140)

Gly Ser Gly Thr Gln Leu Thr,

(SEQ ID NO: 141)

Gly Ser Gly Thr Gln Leu Thr Val,

(SEQ ID NO: 142)

Gly Ser Gly Thr Gln Leu Thr Val Leu,
and

(SEQ ID NO: 143)

Gly Ser Gly Thr Gln Leu Thr Val Leu Ser.

Additional exemplary sequences suitable for Xaaₛ include, without limitation, sequences comprising

Met,
Met Tyr,
Met Tyr Tyr,

(SEQ ID NO: 162)

Met Tyr Tyr Cys,

(SEQ ID NO: 163)

Met Tyr Tyr Cys Ala,

(SEQ ID NO: 164)

Met Tyr Tyr Cys Ala Met,

(SEQ ID NO: 165)

Met Tyr Tyr Cys Ala Met Gly,

(SEQ ID NO: 166)

Met Tyr Tyr Cys Ala Met Gly Ala,

(SEQ ID NO: 167)

Met Tyr Tyr Cys Ala Met Gly Ala Arg,
and

(SEQ ID NO: 168)

Met Tyr Tyr Cys Ala Met Gly Ala Arg Ser.

Exemplary sequences suitable for Xaa, include, without limitation, sequences comprising

Ala
Ala Ser
Ala Ser Thr,

(SEQ ID NO: 145)

Ala Ser Thr Lys,

(SEQ ID NO: 146)

Ala Ser Thr Lys Gly,

(SEQ ID NO: 147)

Ala Ser Thr Lys Gly Pro,

(SEQ ID NO: 148)

Ala Ser Thr Lys Gly Pro Ser,

(SEQ ID NO: 149)

Ala Ser Thr Lys Gly Pro Ser Val,

(SEQ ID NO: 150)

Ala Ser Thr Lys Gly Pro Ser Val Phe,
and

(SEQ ID NO: 151)

Ala Ser Thr Lys Gly Pro Ser Val Phe Pro.

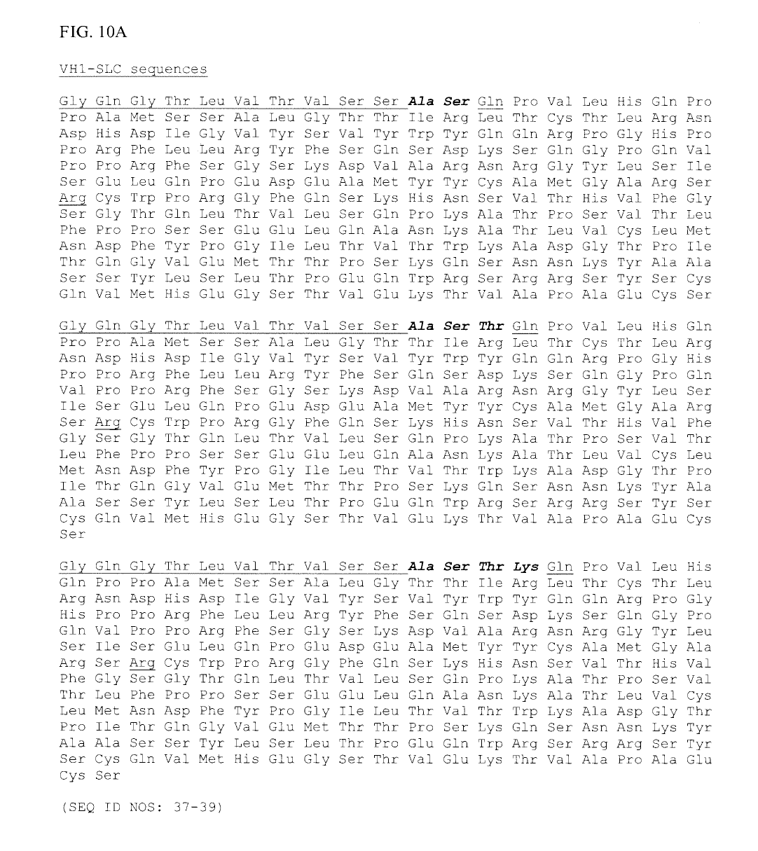

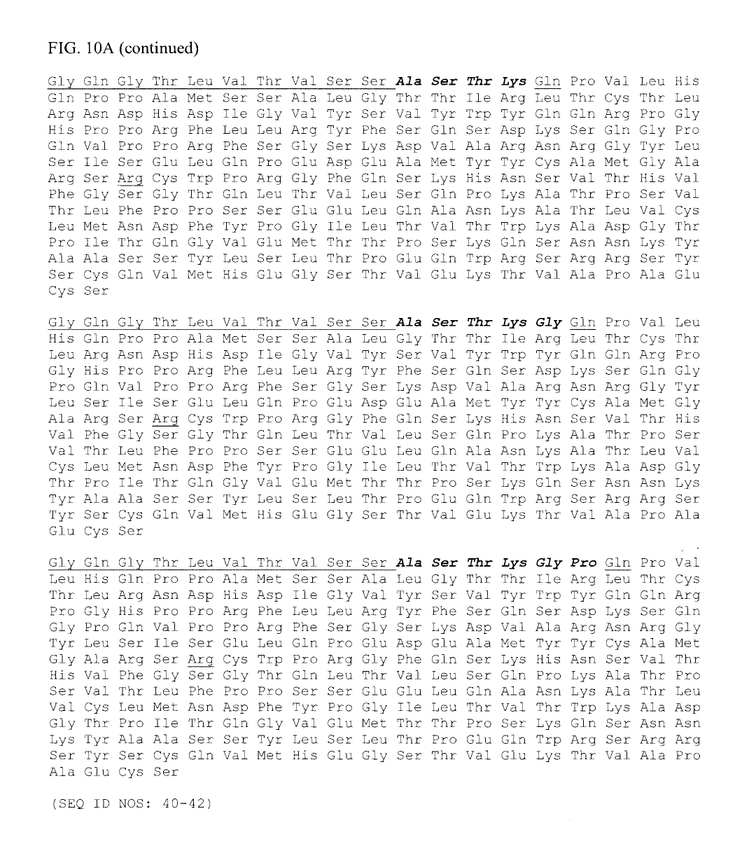

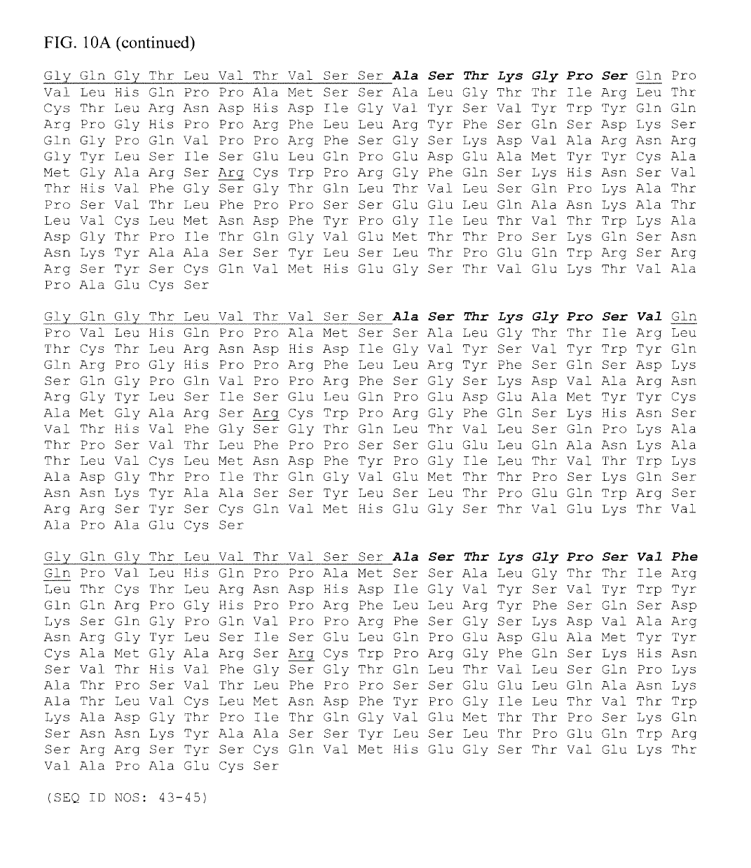

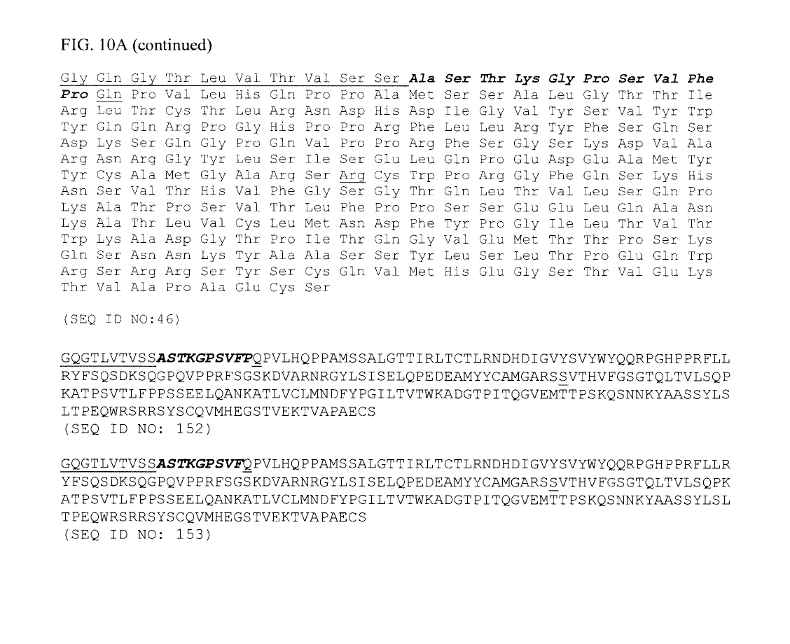

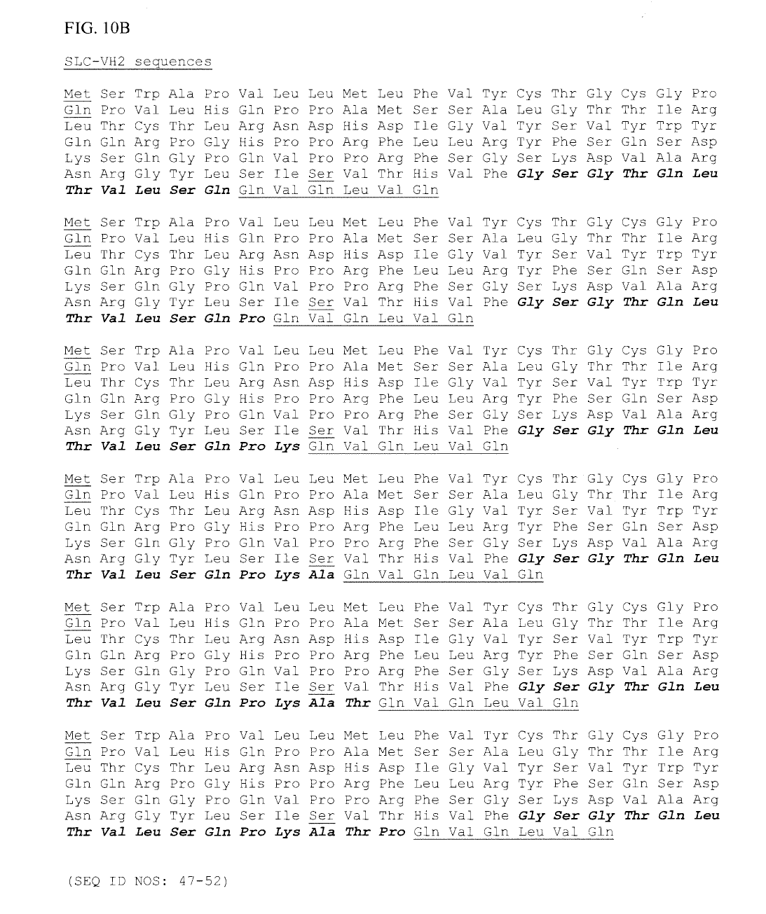

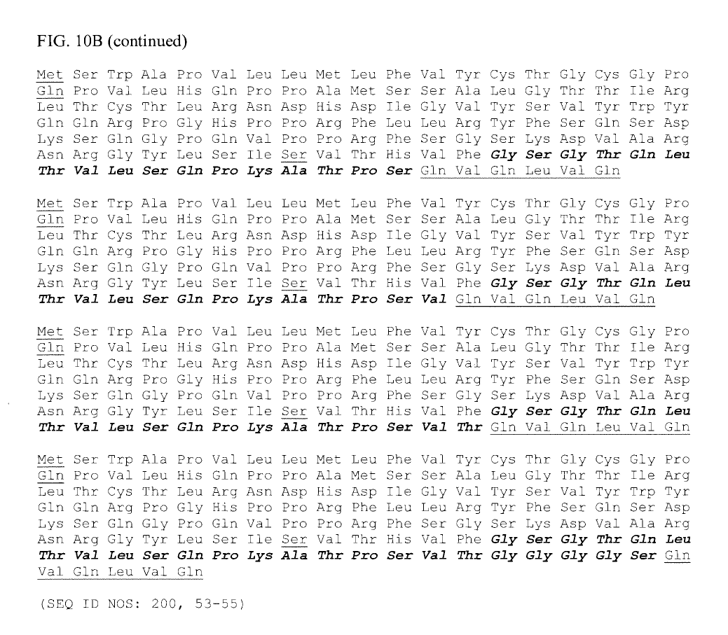

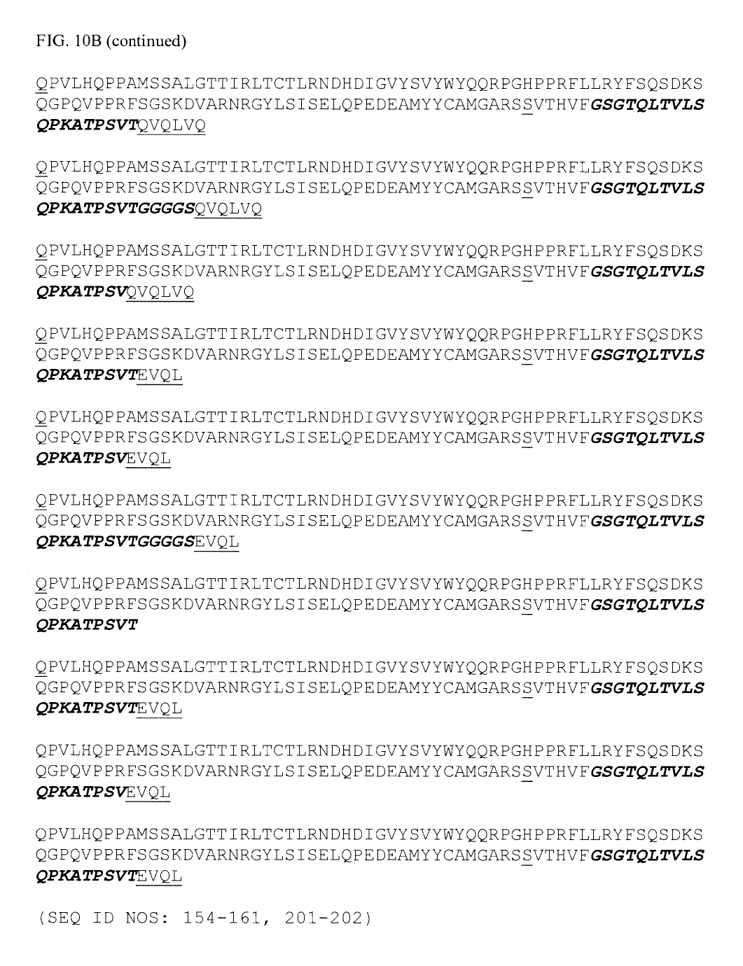

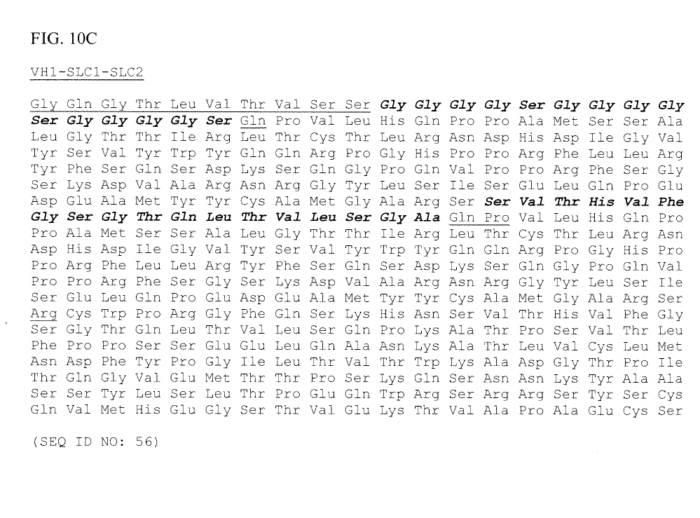

FIGS. 10A-C provide amino acid sequences of exemplary multispecific SVD Surrobody molecules having a variable heavy chain domain sequence linked to a λ-like surrogate light chain domain sequence. FIG. 10A depicts representative sequences that begin with the C-terminal-most region of the heavy chain variable domain sequence (underlined and starting with an underlined Gly at the N-terminus), followed by a linker sequence (bolded and italicized); a VpreB sequence (beginning with an underlined Gln (Q) residue); and a λ5 sequence (beginning with an underlined Arg (R) or Ser (S) residue). FIG. 10B depicts representative sequences that begin with a VpreB sequence (beginning with an underlined Met (M) residue or Gln (Q) residue), followed by a λ5 sequence (beginning with an underlined Ser residue), a linker sequence (bolded and italicized), and the N-terminal-most region of the heavy chain variable domain sequence (underlined and beginning with a Gln (Q) or Glu (E) following the linker sequence; and/or underlined and ending with a Gln (Q) or Leu (L)).

In some embodiments, the first 19 amino acids of the VpreB sequence as shown in FIG. 10B (and underlined in SEQ ID NO:1 of FIG. 5) may be replaced by a heterologous leader sequence (e.g., SEQ ID NO:36). In other embodiments, the molecules of the present invention comprise a VpreB sequence beginning with Gln (Q) as the N-terminal-most residue. The sequences depicted in FIGS. 10A-B may be used in the construction of various bispecific Surrobody molecules, such as those depicted in FIG. 1A. FIG. 10C depicts a representative sequence that begins with the C-terminal-most region of the heavy chain variable domain sequence (underlined and starting with an underlined Gly (G) at the N-terminus), followed by a first linker sequence (bolded, italicized, and beginning with a Gly (G) residue); a first VpreB sequence (beginning with an underlined Gln residue); and a second linker sequence (bolded, italicized, and beginning with a Ser residue), a second VpreB sequence (beginning with an underlined Gln and Pro residue), and a λ5 sequence (beginning with an underlined Arg residue). The sequence depicted in FIG. 10C may be used in the construction of various bispecific Surrobody molecules, such as those depicted in FIG. 1B. As further depicted in FIGS. 1A-B and 12, part of the linker sequence between an N-terminal VpreB sequence and a C-terminal heavy chain variable domain sequence shown in FIGS. 10B and C may include amino acid residues from λ5.

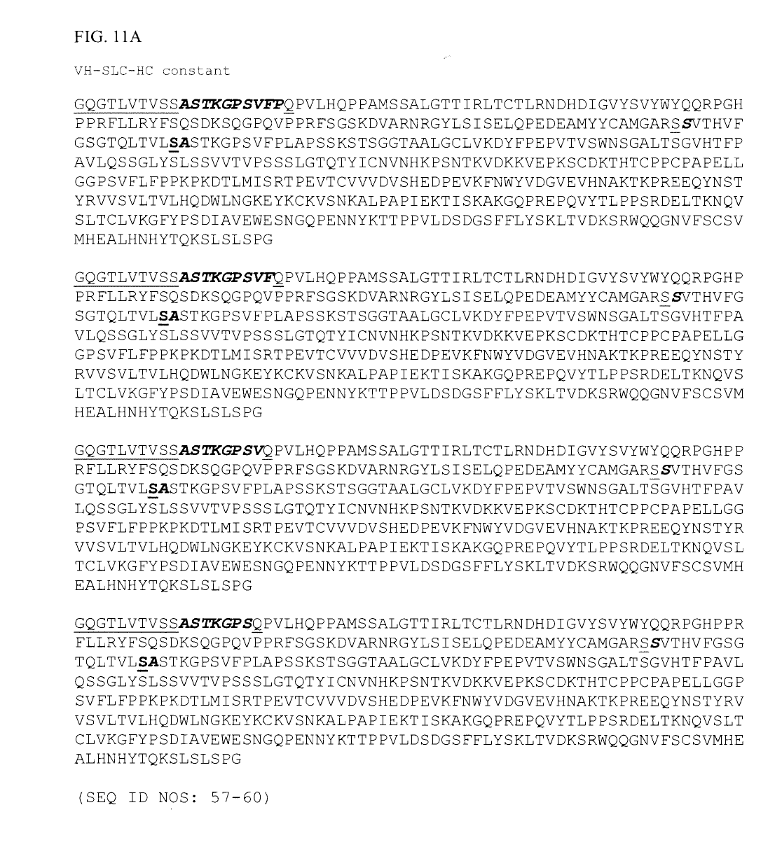

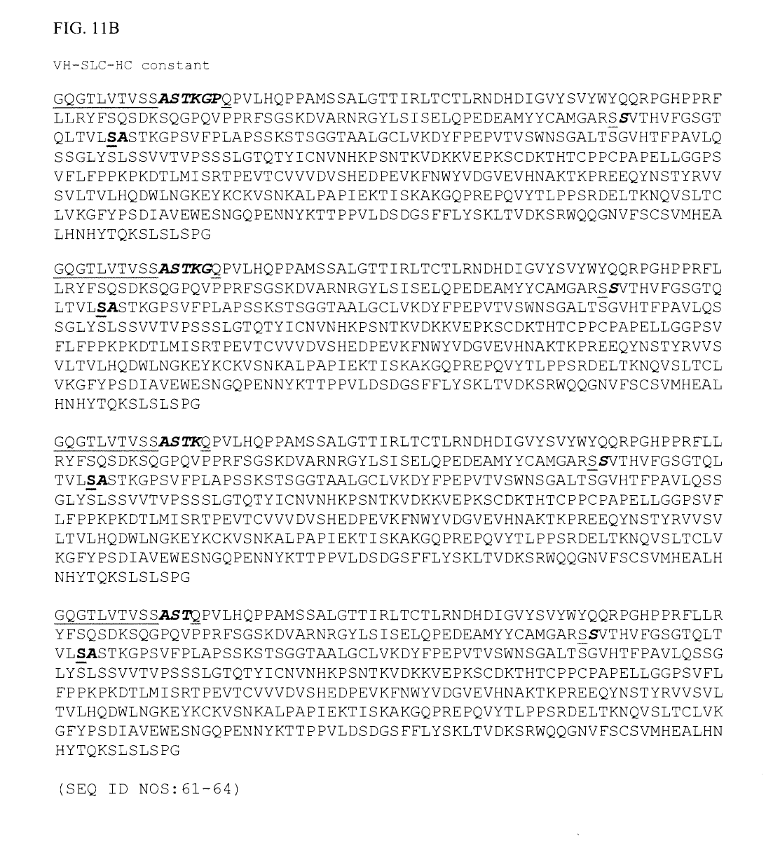

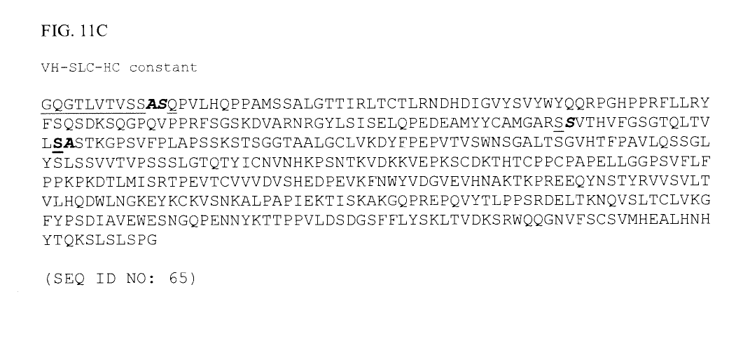

FIG. 11A-C provide amino acid sequences of exemplary multispecific cross-complemented Surrobody molecules having an antibody heavy chain variable domain sequence linked to a VpreB sequence, wherein the VpreB sequence is linked to an antibody heavy chain constant domain sequence. FIG. 11A-C depicts representative sequences that begin with the C-terminal-most region of the heavy chain variable domain sequence (underlined and starting with an underlined G (Gly) at the N-teiminus), followed by a linker sequence (bolded and italicized); a VpreB sequence (beginning with an underlined Q (Gln) residue and ending with an underlined S (Ser) residue); a λ5 sequence (beginning with a bolded S (Ser) residue and ending with an underlined and bolded Ser (S) residue), and an antibody heavy chain constant domain sequence (beginning with a bolded and italicized Ala (A) residue). In some embodiments, the λ5 sequence may be omitted from the sequence such that the C-terminal Ser residue of the VpreB sequence is immediately followed by the first residue of the antibody heavy chain constant domain sequence, e.g., Ala (A) in FIG. 11A-C. FIG. 11A-C provides an exemplary antibody heavy chain constant domain sequence corresponding to the yl constant region (CH1-CH2-CH3) lacking a C-terminal Lys (K) residue (“des-Lys”). However, those of skill in the art will appreciate that other constant domain sequences are suitable for use in the present invention. The antibody or immunoglobulin constant domain sequence can be derived from any of the five types of immunoglobulin heavy chains: γ, δ, α, and ε, which correspond to the five classes of immunoglobulins: IgG, IgD, IgA, IgM, and IgE, respectively.

(ii) κ-Like Surrogate Light Chains

Specific examples of κ-like Surrobodies include polypeptides in which a Vκ-like sequence, including fragments and variants of the native sequences, is conjugated to a JCκ sequence, including fragments and variants of the native sequence. Representative fusions of this type are illustrated in U.S. Patent Publication No. 2001-0062950, and Xu et al., J. Mol. Biol. 2010, 397, 352-360, the entire disclosures of which are expressly incorporated by reference herein.

Various heterodimeric surrogate κ light chain deletion variants may be used as surrogate light chains. In the “full length” construct, both the Vκ-like and JCκ sequence retains the C- and N-terminal extensions (tails), respectively. In the dJ variant, the N-terminal extension of JCκ has been deleted. In the dVκ tail variants, the C-terminal extension of the Vκ-like sequence had been removed but the N-terminal extension of JCκ is retained. In the “short kappa” variant, both the C-terminal tail of the Vκ-like sequence and the N-terminal extension of the JCκ sequence are retained. Single chain constructs may be made between the full length sequences and any of the deletion variants in any combination, e.g., full length single chain, full length Vκ-like and dJ single chain, full length JCκ and dVκ, etc.

Specific examples of the polypeptide constructs herein include polypeptides in which a Vκ-like and/or JCκ sequence is associated with an antibody heavy chain, or a fragment thereof. In the κ-like surrogate light chain constructs of the present invention, the Vκ-like polypeptide and/or the JCκ polypeptide may contain the C- and N-terminal extensions, respectively, that are not present in similar antibody sequences. Alternatively, part or whole of the extension(s) can be removed from the κ-like surrogate light chain constructs herein.

Other κ-like surrogate light chain constructs, which can be used individually or can be further derivatized and/or associated with additional heterologous sequences, such as antibody heavy chain sequences, such as a full-length antibody heavy chain or a fragment thereof.

While the C- and N-terminal extensions of the Vκ-like polypeptide and/or the JCκ polypeptide do not need to be present in the constructs of the present invention, it is advantageous to retain at least a part of at least one of such appendages, because they provide a unique opportunity to create combinatorial functional diversity, either by linear extensions or, for example, in the form of constrained diversity, as a result of screening loop libraries, as described in WO/2010/006286 published on Jan. 14, 2010 and incorporated herein by reference in its entirety. In addition, the “tail” portions of the Vκ-like polypeptide and/or the JCκ polypeptide can be fused to other peptides and/or polypeptides, to provide for various desired properties, such as, for example, enhanced binding, additional binding specificities, enhanced pK, improved half-life, reduced half-life, cell surface anchoring, enhancement of cellular translocation, dominant negative activities, etc. Specific functional tail extensions are further discussed in WO/2010/151808 published on Dec. 29, 2010 and incorporated herein by reference in its entirety.

If desired, the constructs of the present invention can be engineered, for example, by incorporating or appending known sequences or sequence motifs from the CDR1, CDR2 and/or CDR3 regions of antibodies, including known therapeutic antibodies into the CDR1, CDR2 and/or CDR3 analogous regions of the κ-like surrogate light chain sequences. This allows the creation of molecules that are not antibodies, but will exhibit binding specificities and affinities similar to or superior over those of a known therapeutic antibody.

As Vκ-like and the JCκ genes encode polypeptides that can function as independent proteins and function as surrogate light chains, surrogate-like light chains can be engineered from true light chains and be used in every previous application proposed for engineered true surrogate light chains. This can be accomplished by expressing the variable light region to contain a peptidic extension analogous to either the VpreB or Vκ-like gene. Similarly the constant region can be engineered to resemble either the λ5 or JCκ genes and their peptidic extensions. Furthermore any chimeras or heterodimeric partnered combinations are within the scope herein.

In one other aspect, the present invention contemplates multispecific Surrobody molecules comprising surrogate light chain (SLC) domains that have κ-like SLC polypeptides. In one embodiment, the κ-like SLC polypeptide comprises a Vκ-like sequence and/or a JCκ sequence. In another embodiment, the Vκ-like sequence is selected from the group consisting of SEQ ID NOS: 12-24, and fragments and variants thereof. In one other embodiment, the JCκ sequence is selected from the group consisting of SEQ ID NOS:26-39, and fragments and variants thereof. The κ-like SLC domain may be a Vκ-like sequence conjugated to a JCκ sequence. The conjugate may be a fusion. In another embodiment, the fusion takes place at or around the CDR3 analogous regions of said Vκ-like sequence and said JCκ sequence respectively. In one embodiment, the invention contemplates a κ-like SLC construct, wherein said Vκ-like sequence and said JCκ sequence are connected by a covalent linker.

In one embodiment, the invention provides a κ-like SLC construct, wherein said Vκ-like sequence is non-covalently associated with said JCκ sequence. In one embodiment, the invention provides a κ-like SLC construct wherein the conjugate of said Vκ-like sequence and JCκ sequence is non-covalently associated with an antibody heavy chain sequence.

The multispecific Surrobody molecules of the present invention may contain a Vκ-like sequence and/or a JCκ sequence. The multispecific Surrobody molecules may have a Vκ-like polypeptide conjugated to an antibody heavy chain domain. In one embodiment, the conjugate is a fusion. The fusions may have particular junctions or linkage regions between the Vκ-like sequence polypeptide and the heavy chain polypeptide. Exemplary sequences suitable to link the Vλ-like sequence and the heavy chain include, without limitation, sequences comprising

Ala Ser,
Ala Ser Thr,

(SEQ ID NO: 112)

Ala Ser Thr Lys,

(SEQ ID NO: 113)

Ala Ser Thr Lys Gly,

(SEQ ID NO: 114)

Ala Ser Thr Lys Gly Pro,

(SEQ ID NO: 115)

Ala Ser Thr Lys Gly Pro Ser,

(SEQ ID NO: 116)

Ala Ser Thr Lys Gly Pro Ser Val,

(SEQ ID NO: 117)

Ala Ser Thr Lys Gly Pro Ser Val Phe,
and

(SEQ ID NO: 118)

Ala Ser Thr Lys Gly Pro Ser Val Phe Pro.

In one embodiment, the linking sequence links an antibody variable heavy chain domain with a Vκ-like domain. The sequence may be a CH1 amino acid sequence. In another embodiment, the linking sequence is carboxy-terminal to the heavy chain variable domain and/or amino-terminal to the Vκ-like domain.

In another embodiment, the linking sequence is amino-terminal to the heavy chain variable domain and/or carboxy-terminal to the Vκ-like domain.

In one other aspect, the multispecific Surrobody molecules will have amino acid junction or linkage regions comprising sequences from an antibody heavy chain variable (HCV) domain, an antibody heavy chain constant domain, and a Vκ-like domain. In one embodiment, the HCV domain is amino-terminal to the Vλ-like domain and separated by the heavy chain constant domain sequence. Exemplary sequences suitable as linkage regions include, without limitation, sequences comprising

(SEQ ID NO: 67)

Xaa_{g} Ala Ser Xaaₕ,

(SEQ ID NO: 68)

Xaa_{g} Ala Ser Thr Xaaₕ,

(SEQ ID NO: 69)

Xaa_{g} Ala Ser Thr Lys Xaaₕ,

(SEQ ID NO: 70)

Xaa_{g} Ala Ser Thr Lys Gly Xaaₕ,

(SEQ ID NO: 71)

Xaa_{g} Ala Ser Thr Lys Gly Pro Xaaₕ,

(SEQ ID NO: 72)

Xaa_{g} Ala Ser Thr Lys Gly Pro Ser Xaaₕ,

(SEQ ID NO: 73)

Xaa_{g} Ala Ser Thr Lys Gly Pro Ser Val Xaaₕ,

(SEQ ID NO: 74)

Xaa_{g} Ala Ser Thr Lys Gly Pro Ser Val Phe Xaaₕ,
and

(SEQ ID NO: 75)

Xaa_{g} Ala Ser Thr Lys Gly Pro Ser Val Phe Pro Xaaₕ.

The underlined region is an antibody heavy chain CH1 sequence. Xaa is any amino acid, g is 1 to 10 amino acids, and h is 1 to 10 amino acids. Xaa_{g} may be an antibody heavy chain variable domain sequence. Xaaₖ may be a Vκ-like sequence. Exemplary sequences suitable for Xaa_{g} include, without limitation, sequences comprising

Ser,
Ser Ser,
Val Ser Ser,

(SEQ ID NO: 76)

Thr Val Ser Ser,

(SEQ ID NO: 77)

Val Thr Val Ser Ser,

(SEQ ID NO: 78)

Leu Val Thr Val Ser Ser,

(SEQ ID NO: 79)

Thr Leu Val Thr Val Ser Ser,

(SEQ ID NO: 80)

Gly Thr Leu Val Thr Val Ser Ser,

(SEQ ID NO: 81)

Gln Gly Thr Leu Val Thr Val Ser Ser,
and

(SEQ ID NO: 82)

Gly Gln Gly Thr Leu Val Thr Val Ser Ser.

In another embodiment, the HCV domain is carboxy-terminal to the Vλ-like domain. Exemplary sequences suitable as linkage regions include, without limitation, sequences comprising Xaa_{q}-X-Xaaᵣ.

X is a linker sequence. Xaa is any amino acid, q is 1 to 10 amino acids, and r is 1 to 10 amino acids. Xaa_{q} may be a Vκ-like sequence. Xaaᵣ may be an antibody heavy chain variable sequence. Exemplary sequences suitable for Xaaᵣ include, without limitation,

| | |
|---|---|
| Gln, | |
| Gln Val, | |
| Gln Val Gln, | |
| Gln Val Gln Leu, | (SEQ ID NO: 105) |
| Gln Val Gln Leu Val, | (SEQ ID NO: 106) |
| and | |
| Gln Val Gln Leu Val Gln. | (SEQ ID NO: 107) |

In one embodiment, the Xaaᵣ is a sequence from a heavy chain germline including, without limitation, V_{H}1 1-3 1-02, and V_{H}1 1-2 1-e. Other exemplary germline sequences suitable for Xaaᵣ include, without limitation, Glu Val, Glu Val Gln, and Glu Val Gln Leu (SEQ ID NO: 105).

In one aspect, the present invention provides multispecific Surrobody molecules that include a single chain Surrobody fragment (scSv) having a κ-like surrogate light chain sequence. In one embodiment, the κ-like SLC sequence is a Vκ-like sequence. The scSv may be an antibody heavy chain variable domain conjugated to a first Vκ-like polypeptide having a first Vκ-like domain. In one embodiment, the conjugate is a fusion. The fusions may have particular junctions or linkage regions between the first SLC polypeptide and the heavy chain polypeptide. In one embodiment, the linking sequence contains a (G4S)3 sequence (Gly Gly Gly Gly Ser Gly Gly Gly Gly Ser Gly Gly Gly Gly Ser—SEQ ID NO: 119). The (G4S)3 sequence may be located carboxy-terminal to the heavy chain variable domain and amino-terminal to the Vκ-like domain. In such cases, suitable linkage regions include, without limitation, sequences comprising Xaaᵣ (Gly4Ser)3 Xaa_{q} (SEQ ID NO: 150). Xaaᵣ may be an antibody heavy chain variable domain sequence. Exemplary sequences suitable for Xaaᵣ include, without limitation, sequences comprising

Ser,
Ser Ser,
Val Ser Ser,

(SEQ ID NO: 76)

Thr Val Ser Ser,

(SEQ ID NO: 77)

Val Thr Val Ser Ser,

(SEQ ID NO: 78)

Leu Val Thr Val Ser Ser,

(SEQ ID NO: 79)

Thr Leu Val Thr Val Ser Ser,

(SEQ ID NO: 80)

Gly Thr Leu Val Thr Val Ser Ser,

(SEQ ID NO: 81)

Gln Gly Thr Leu Val Thr Val Ser Ser,
and

(SEQ ID NO: 82)

Gly Gln Gly Thr Leu Val Thr Val Ser Ser.

Xaa_{q} may be a Vκ-like sequence. The scSv molecules may also have a second Vκ-like polypeptide with a second Vκ-like domain conjugated to the first Vκ-like polypeptide. In one embodiment, the conjugate is a fusion. The second Vκ-like polypeptide may be located carboxy-terminal to the first Vκ-like polypeptide. The fusions may have particular junctions or linkage regions between the first and the second Vκ-like polypeptides. In one embodiment, the linking sequence contains a Gly Ala (GA) sequence. The GA sequence may be located carboxy-terminal to the first Vκ-like polypeptide and amino-terminal to the second Vκ-like polypeptide. In such cases, suitable linkage regions include, without limitation, sequences comprising Xaa_{q} Gly Ala Xaa (SEQ ID NO:). Xaa_{q} is a first Vκ-like domain sequence. Xaa is any amino acid, n is 1 to 10 amino acids, and p is 1 to 10 amino acids. Xaa_{q} may be a Vκ-like sequence. Xaa may be a Vκ-like sequence.

b. Dimerization or Multimerization Domains

The polypeptide chains of the multispecific molecules described herein may have a multimerization or dimerization domain. Such domains may be conjugated to the other parts of the chain, such as the antibody variable heavy chain domain and/or surrogate light chain domain. In one embodiment, the conjugate is a fusion. Examples of multimerization domains include, without limitation, the immunoglobulin sequences or portions thereof, leucine zippers, complementary hydrophobic regions, complementary hydrophilic regions, compatible protein-protein interaction domains including, without limitation, an R subunit of PKA and an anchoring domain (AD), a free thiol that forms an intermolecular disulfide bond between two molecules, and a protuberance-into-cavity (i.e., knob into hole) and a compensatory cavity of identical or similar size that form stable multimers. The multimerization domain, for example, can be an immunoglobulin constant region. The immunoglobulin sequence can be an immunoglobulin constant domain, such as the Fc domain or portions thereof from IgG1, IgG2, IgG3 or IgG4 subtypes, IgA, IgE, IgD and IgM (Shepard et al. US Published App. 20100055093).

c. Heterologous Leader Sequences

The present invention provides heterologous leader sequences that improve the efficiency of recombinant expression of the surrogate light chain polypeptides that may be used to form the multispecific Surrobody molecules described herein. In one aspect, the present invention provides isolated nucleic acid molecules encoding a surrogate light chain (SLC) polypeptide or SLC construct containing an SLC polypeptide, wherein the native secretory leader sequence of the polypeptide is replaced by a heterologous secretory leader sequence. In one embodiment, the SLC polypeptide includes a VpreB polypeptide, a λ5 polypeptide, or fragments or variants thereof. In another embodiment, the VpreB polypeptide is selected from the group consisting of a native VpreB 1 sequence, a native VpreB2 sequence, a native VpreB3 sequence, and fragments and variants thereof. In some embodiments, the native VpreB sequence is selected from the group consisting of human VpreB 1 of SEQ ID NO: 1, mouse VpreB2 of SEQ ID NOS: 2 and 3, human VpreB3 of SEQ ID NO: 4, human VpreB-like polypeptide of SEQ ID NO:5, human VpreB dTail polypeptide of SEQ ID NO:6 and fragments and variants thereof. In one other embodiment, the λ5 polypeptide is selected from the group consisting of a murine λ5-like of SEQ ID NO: 7; a human λ5-like polypeptide of SEQ ID NO: 8, a human λ5 dTail polypeptide of SEQ ID NO:9, and fragments and variants thereof. In another embodiment, the SLC polypeptide includes a Vκ-like polypeptide, a JCκ polypeptide, or fragments or variants thereof. In one other embodiment, the Vκ-like polypeptide sequence is selected from the group consisting of SEQ ID NOS: 12-24, and fragments and variants thereof. In some embodiments, the JCκ polypeptide sequence is selected from the group consisting of SEQ ID NOS:26-39, and fragments and variants thereof.

In another aspect, the present invention provides isolated nucleic acid molecules encoding a surrogate light chain (SLC) polypeptide, wherein the native secretory leader sequence of the polypeptide is replaced by a heterologous secretory leader sequence and the SLC polypeptide includes an SLC polypeptide fusion, or fragments or variants thereof. In one embodiment, the SLC fusion includes a VpreB-λ5 polypeptide fusion, or fragments or variants thereof. In another embodiment, the fusion of the VpreB polypeptide sequence and λ5 polypeptide sequence takes place at or around the CDR3 analogous regions of the VpreB sequence and the λ5 sequence respectively. In one other embodiment, the VpreB polypeptide sequence is linked at its carboxy terminus to the amino terminus of the λ5 polypeptide sequence. In one embodiment, the SLC fusion includes a Vκ-like-JCκ polypeptide fusion, or fragments or variants thereof. In another embodiment, the fusion of the Vκ-like polypeptide sequence and JCκ polypeptide sequence takes place at or around the CDR3 analogous regions of the Vκ-like sequence and the JCκ sequence respectively. In one other embodiment, the Vκ-like polypeptide sequence is fused at its carboxy terminus to the amino terminus of the JCκ polypeptide sequence.

In all embodiments, the heterologous secretory leader sequence may be a leader sequence of a secreted polypeptide selected from the group consisting of antibodies, cytokines, lymphokines, monokines, chemokines, polypeptide hormones, digestive enzymes, and components of the extracellular matrix. In one embodiment, the cytokine may be selected from the group consisting of growth hormone, such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β (TNF-α and -β); mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and —II; erythropoietin (EPO); osteoinductive factors; interferons such as interferon-α, -β and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-la, IL-2, IL-3, IL-4, IL-5, IL-6, IL-7, IL-8, IL-9, IL-10, IL-11, IL-12; a tumor necrosis factor such as TNF-α or TNF-β; MIP-1α; MIP-1β; and other polypeptide factors including LIF and kit ligand (KL).

In all embodiments, the secretory leader sequence may be selected from the group consisting of leader sequences of human and non-human mammalian albumin, transferrin, CD36, growth hormone, tissue plasminogen activator (t-PA), erythropoietin (EPO), and neublastin.

In all embodiments, the secretory leader sequence may be a synthetic sequence.

In all embodiments, the secretory leader sequence may be a consensus sequence of native secretory leader sequences.

The murine Ig kappa leader sequence may be used (METDTLLLWVLLLWVPGSTG—SEQ ID NO:36) as a heterologous leader sequence.

In all embodiments, the present invention provides an isolated nucleic acid molecule encoding a surrogate light chain (SLC) construct.

In one aspect, the present invention provides vectors and recombinant host cells. In all embodiments, the vectors may contain a nucleic acid molecule described herein. In all embodiments, the recombinant host cells may be transformed with a nucleic acid described herein.

In another aspect, the present invention provides methods for the expression of a surrogate light chain (SLC) polypeptide or SLC construct in a recombinant host cell. In one embodiment, the method includes the step of transforming the recombinant host cell with a nucleic acid molecule encoding an SLC polypeptide or SLC construct, wherein the native secretory leader sequence of the polypeptide is replaced by a heterologous secretory leader sequence. In another embodiment, the recombinant host cell is an eukaryotic cell. In one other embodiment, the recombinant host cell is a Chinese Hamster Ovary (CHO) cell or a human embryonic kidney (HEK) 293 cell. In some embodiments, the SLC polypeptide or SLC construct is selected from the group consisting of an SLC polypeptide comprising one or more of a VpreB polypeptide, a λ5 polypeptide, a VpreB-λ5 polypeptide fusion, a Vκ-like polypeptide, a JCκ polypeptide, and a Vκ-like-JCκ polypeptide fusion.

The present invention provides nucleic acid and polypeptide constructs for producing surrogate light chain constructs in higher yields than when such constructs are produced from sequences that comprise an endogenous leader VpreB leader sequence and/or λ5 leader sequence, or an endogenous Vκ-like leader sequence and/or JCκ leader sequence. The present invention also provides vectors, host cells and methods for producing surrogate light chain constructs in higher yields than when such constructs are produced from DNA sequences that include the coding sequence of the endogenous leader of VpreB and/or λ5, or the endogenous leader of Vκ-like and/or JCκ, or without an endogenous leader sequence. The higher yields are achieved by replacing at least one endogenous secretory leader sequence with a heterologous leader sequence of the invention. Accordingly, the present invention provides surrogate light chains and surrogate light chain constructs comprising heterologous leader sequences.

Preferably, the expression level achieved by a heterologous leader peptide is at least about 5% higher, at least about 10% higher, at least about 20% higher, at least about 30% higher, at least about 40% higher, or at least about 50% higher than the expression level achieved by using a homologous leader sequence, when expression is conducted under essentially the same conditions.

In the present invention, a heterologous leader sequence is fused to the amino terminus of a surrogate light chain polypeptide, in place of the native VpreB leader sequence and/or the native λ5 leader sequence, or a κ-like surrogate light chain polypeptide, in place of the native Vκ-like leader sequence and/or the native JCκ leader sequence. The inventors have discovered that certain heterologous leader sequences function surprisingly well, in contrast to the native leader sequence of the surrogate light chain during the production of surrogate light chain constructs, comprising a surrogate light chain sequence (VpreB/λ5 or Vκ-like/JCκ sequences either fused together or non-covalently associated) and an antibody heavy chain sequence.

According to the present invention, the heterologous leader sequence can be any leader sequence from a highly translated protein, including leader sequences of antibody light chains and human and non-human mammalian secreted proteins. Secreted proteins are included and their sequences are available from public databases, such as Swiss-Prot, UniProt, TrEMBL, RefSeq, Ensembl and CBI-Gene. In addition, SPD, a web based secreted protein database is a resource for such sequences, available at http://spd.ebi.pku.edu.cn. (See, Chen et al., Nucleic Acids Res., 2005, 33:D169-D173). Such secreted proteins include, without limitation, antibodies, cytokines, lymphokines, monokines, chemokines, polypeptide hormones, digestive enzymes, and components of the extracellular matrix. Further leader sequences suitable for use in the constructs of the present invention are included in publicly available signal peptide databases, such as, the SPdb signal peptide database, accessible at http://proline.bis.nus.cdu.sq/spdb (See, Choo et al., BMC Bioinformutics 2005, 6:249).

Specific examples of suitable heterologous leader sequences include, without limitation, leader sequences of human and non-human mammalian albumin, transferrin, CD36, growth hormone, tissue plasminogen activator (t-PA), erythropoietin (EPO), neublastin leader sequences and leader peptides from other secreted human and non-human proteins.

When heterologous leader sequences are present in i) both a VpreB and a λ5 surrogate light chain construct, or ii) both a Vκ-like and a JCκ surrogate light chain construct, each heterologous leader sequence in i) or ii) may be identical to the other or may be different from the other.

In addition to signal peptides from native proteins, the heterologous leader sequences of the present invention include synthetic and consensus leader sequences, which can be designed to further improve the performance of leader sequences occurring in nature, and specifically adapted for best performance in the host organism used for the expression of the surrogate light chain constructs of the present invention.

The multispecific binding proteins of the present invention may be provided in formats that provide additional functionality. As described in Xu et al., J. Mol. Biol. 2010, 397, 352-360, various functional components may be added to Surrobody formats, including cytokines and antibody fragments. It is possible to utilize this approach in the multispecific binding protein formats of the present invention. For example, any of the polypeptide chains or heteromeric bispecific binding proteins containing such chains that are described herein may further include a heterologous polypeptide having a certain function. In one embodiment, the heterologous polypeptpide may be a cytokine, which can provide additional functionality. In another embodiment, the heterologous polypeptpide may be an antibody fragment, which can provide additional specificity. For example, a polypeptide chain containing a VpreB sequence may further include a heterologous sequence that provides additional functionality. Alternatively, for structures using a VpreB sequence, the heterologous sequence providing additional functionality is conjugated to the N-terminus of a polypeptide sequence that is normally conjugated to the C-terminus of the VpreB sequence. In one embodiment, the N-terminus of a λ5 or light chain constant region sequence is conjugated to the C terminus of the sequence providing additional functionality.

Antibody Heavy Chain Variable s u Nces

In one aspect, the present invention provides multispecific SVD molecules suitable for use with any polypeptide target. As a result, the sequence of a heavy chain variable domain (or any functional fragment thereof) from an antibody specific for any target may be incorporated into one of the multispecific SVD structures described herein. In one embodiment, the molecules comprise heavy chain variable domain sequences from Placenta growth factor(PIGF) (SEQ ID NO:205) or hepatocyte growth factor (HGF) (SEQ ID NO:206) (see also, Example 1).

PlGF

(SEQ ID NO: 205)

QVQLVQSGAEVKKPGSSVKVSCKASGGTFSTYGISWVRQAPGQGLEWVG
WITPITGHTTYAQKFQGRVTITADKSTSTAYMELSSLRSEDTAVYYCAR
DGATIWNAGLDYWGQGTLVTVSS
HGF

(SEQ ID NO: 206)

QVQLVQSGAEVKKAPGASVKVSCKASGYTFSNYGMHWVRQAPGQGLEWM
GGINVNSGGPNYAQKFQGRVTMTRVDTSISTAYMELSRLRSDDTAVYYCAR
VGWSLDSSRGSGMDYWGQGTLVTVSS

Preparation of Surrogate Light Chain Constructs

Nucleic acids encoding the surrogate light chain constructs, e.g. VpreB and λ5 polypeptides or Vκ-like or JCκ polypeptides, can be isolated from natural sources, e.g. developing B cells and/or obtained by synthetic or semi-synthetic methods. Once this DNA has been identified and isolated or otherwise produced, it can be ligated into a replicable vector for further cloning or for expression.

Cloning and expression vectors that can be used for expressing the coding sequences of the polypeptides herein are well known in the art and are commercially available. The vector components generally include, but are not limited to, one or more of the following: a signal sequence, an origin of replication, one or more marker genes, an enhancer element, a promoter, and a transcription termination sequence. Suitable host cells for cloning or expressing the DNA encoding the surrogate light chain constructs in the vectors herein are prokaryote, yeast, or higher eukaryote (mammalian) cells, mammalian cells are being preferred.

Examples of suitable mammalian host cell lines include, without limitation, monkey kidney CV1 line transformed bySV40 (COS-7, ATCC CRL 1651); human embryonic kidney (HEK) line 293 (HEK 293 cells) subcloned for growth in suspension culture, Graham et al, J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and Wa human hepatoma line (Hep G2).

For use in mammalian cells, the control functions on the expression vectors are often provided by viral material. Thus, commonly used promoters can be derived from the genomes of polyoma, Adenovirus2, retroviruses, cytomegalovirus, and Simian Virus 40 (SV40). Other promoters, such as the β-actin protomer, originate from heterologous sources. Examples of suitable promoters include, without limitation, the early and late promoters of SV40 virus (Fiers et al., Nature, 273: 113 (1978)), the immediate early promoter of the human cytomegalovirus (Greenaway et al., Gene, 18: 355-360 (1982)), and promoter and/or control sequences normally associated with the desired gene sequence, provided such control sequences are compatible with the host cell system.

Transcription of a DNA encoding a desired heterologous polypeptide by higher eukaryotes is increased by inserting an enhancer sequence into the vector. The enhancer is a cis-acting element of DNA, usually about from 10 to 300 bp, that acts on a promoter to enhance its transcription-initiation activity. Enhancers are relatively orientation and position independent, but preferably are located upstream of the promoter sequence present in the expression vector. The enhancer might originate from the same source as the promoter, such as, for example, from a eukaryotic cell virus, e.g. the SV40 enhancer on the late side of the replication origin (bp 100-270), the cytomegalovirus early promoter enhancer, the polyoma enhancer on the late side of the replication origin, and adenovirus enhancers.

Expression vectors used in mammalian host cells also contain polyadenylation sites, such as those derived from viruses such as, e.g., the SV40 (early and late) or HBV.

An origin of replication may be provided either by construction of the vector to include an exogenous origin, such as may be derived from SV40 or other viral (e.g., Polyoma, Adeno, VSV, BPV) source, or may be provided by the host cell.

The expression vectors usually contain a selectable marker that encodes a protein necessary for the survival or growth of a host cell transformed with the vector. Examples of suitable selectable markers for mammalian cells include dihydrofolate reductase (DHFR), thymidine kinase (TK), and neomycin.

Suitable mammalian expression vectors are well known in the art and commercially available. Thus, for example, the surrogate light chain constructs of the present invention can be produced in mammalian host cells using a pCI expression vector (Promega), carrying the human cytomegalovirus (CMV) immediate-early enhancer/promoter region to promote constitutive expression of a DNA insert. The vector may also be the pTT5 expression vector (National Research Council, Canada). The vector can contain a neomycin phosphotransferase gene as a selectable marker.

The surrogate light chain constructs of the present invention can also be produced in bacterial host cells. Control elements for use in bacterial systems include promoters, optionally containing operator sequences, and ribosome binding sites. Suitable promoters include, without limitation, galactose (gal), lactose (lac), maltose, tryptophan (trp), β-lactamase promoters, bacteriophage λ and T7 promoters. In addition, synthetic promoters can be used, such as the tac promoter. Promoters for use in bacterial systems also generally contain a Shine-Dalgarno (SD) sequence operably linked to the DNA encoding the Fab molecule. The origin of replication from the plasmid pBR322 is suitable for most Gram-negative bacteria.

The coding sequences of the individual chains within a multi-chain construct comprising antibody surrogate light chain sequences can be present in the same expression vector, under control of separate regulatory sequences, or in separate expression vectors, used to co-transfect a desired host cells, including eukaryotic and prokaryotic hosts. Thus, multiple genes can be coexpressed using the Duet™ vectors commercially available from Novagen.

The transformed host cells may be cultured in a variety of media. Commercially available media for culturing mammalian host cells include Ham's F10 (Sigma), Minimal Essential Medium ((MEM), (Sigma), RPMI-1640 (Sigma), and Dulbecco's Modified Eagle's Medium ((DMEM), Sigma). In addition, any of the media described in Ham et al., Meth. Enz. 58:44 (1979) and Barnes et al., Anal. Biochem. 102:255 (1980) may be used as culture media for the host cells. The culture conditions, such as temperature, pH, and the like, are those previously used with the host cell selected for expression, and are included in the manufacturer's instructions or will otherwise be apparent to the ordinarily skilled artisan.

Further suitable media for culturing mammalian, bacterial (e.g. E. coli) or other host cells are also described in standard textbooks, such as, for example, Sambrook et al., supra, or Ausubel et al., supra.

In one aspect, the present invention provides a method for the expression of a surrogate light chain in a recombinant host cell. In one embodiment, the method includes the step of providing a nucleic acid encoding an SLC polypeptide or an SLC fusion polypeptide. In another embodiment, the method includes the step of transforming or transfecting the recombinant host cell with a nucleic acid encoding an SLC polypeptide or SLC fusion polypeptide. In one embodiment, the nucleic acid encoding an SLC fusion polypeptide is a chimeric molecule comprising a first SLC sequence covalently connected to a second SLC sequence, wherein the native secretory leader sequence of the first SLC sequence and/or the second SLC sequence is replaced by a heterologous secretory leader sequence. The first SLC sequence may be a VpreB sequence, a Vκ-like sequence, or a fusion polypeptide thereof. The second SLC sequence may be a λ5 sequence, a JCκ sequence, or a fusion polypeptide thereof.

In one embodiment, a VpreB sequence is covalently connected to a λ5 sequence, wherein the native secretory leader sequence of said VpreB sequence and/or said λ5 sequence is replaced by a heterologous secretory leader sequence. In another embodiment, the VpreB sequence is fused to the λ5 sequence. In one other embodiment, the VpreB sequence is connected to the λ5 sequence through a peptide or polypeptide linker. In one other embodiment, a Vκ-like sequence is covalently connected to a JCκ sequence, wherein the native secretory leader sequence of said Vλ-like sequence and/or said JCκ sequence is replaced by a heterologous secretory leader sequence. In one other embodiment, the Vκ-like sequence is fused to the JCκ sequence. In another embodiment, the Vκ-like sequence is connected to the JCκ sequence through a peptide or polypeptide linker.

In other embodiments, the SLC sequence is covalently connected to an antibody heavy chain sequence.

In all embodiments, the methods of expression may comprise the step of transforming or transfecting a host cell with more than one nucleic acid encoding a surrogate light chain polypeptide, including surrogate light chain polypeptides and/or surrogate light chain fusion polypeptides.

In all embodiments, the methods may further comprise the step of transforming or transfecting a host cell with a nucleic acid encoding an antibody heavy chain.

In one aspect, the present invention provides methods for the expression of surrogate light chain polypeptides and/or surrogate light chain fusion polypeptides having improved yields. In one embodiment, the methods of the present invention utilizing heterologous leader sequences in place of native leader sequences are characterized greater polypeptide expression and yield than methods which do not replace native leader sequences with heterologous leader sequences.

In one embodiment, the recombinant host cell is bacterial cell. In another embodiment, the host cell is a eukaryotic cell. In one embodiment, the recombinant host cell is a Chinese Hamster Ovary (CHO) cell, or a human embryonic kidney (HEK) 293 cell.

In one aspect, the present invention provides host cells containing the nucleic acids described herein. In one embodiment, the invention provides a recombinant host cell transformed with at least one nucleic acid described herein. In one other embodiment, the host cell is transformed with a nucleic acid encoding an SLC fusion, which may or may not include a non-SLC molecule.

In all embodiments, the host cell is further transformed with a nucleic acid encoding an antibody heavy chain.

In all embodiments, the present invention provides vectors that contain the nucleic acids described herein. In all embodiments, the host cell is transformed with at least one vector containing a nucleic acid described herein.

Purification can be performed by methods known in the art. In a preferred embodiment, the surrogate light chain constructs are purified in a 6xHis-tagged form, using the Ni-NTA purification system (Invitrogen).

κ-like SLC molecules can be engineered from existing light chain V genes and light chain constant genes. Light chains are products of gene rearrangement and RNA processing. As the components of the κ-like SLC molecules provide alternative function from unrearranged light chain V genes and rearranged light chain JC genes, it is feasible to engineer similar translated proteins from all remaining kappa and lambda light chain V genes to make Vκ-like molecules and all combinations of the remaining kappa JC rearrangements (4 JCκ-like) and lambda JC rearrangements (4 “J”×10 “constant”==40 JCλ-like). Each one of these engineered molecules can serve purposes similar to those using Vκ-like and JCκ, as well as those contained in PCT Publication WO 2008/118970 published on Oct. 2, 2008 and WO/2010/151808 published on Dec. 29, 2010, with VpreB and λ5, and combinations and chimeras thereof.

The surrogate light chains of the present invention can be used to construct molecules for the prevention and/or treatment of disease. For such applications, molecules containing a surrogate light chain are usually used in the form of pharmaceutical compositions. Techniques and formulations generally may be found in Remington's Pharmaceutical Sciences, 18th Edition, Mack Publishing Co. (Easton, Pa. 1990). See also, Wang and Hanson “Parenteral Formulations of Proteins and Peptides: Stability and Stabilizers,” Journal of Parenteral Science and Technology, Technical Report No. 10, Supp. 42-2S (1988).

Polypeptide-based pharmaceutical compositions are typically formulated in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes {e.g., Zn-protein complexes); and/or non-ionic surfactants such as TWEEN™, PLURONICS™ or polyethylene glycol (PEG).

The molecules also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, supra.

The molecules containing surrogate light chains disclosed herein may also be formulated as immunoliposomes. Liposomes containing the molecules are prepared by methods known in the art, such as described in Epstein et al, Proc. Natl. Acad. Sci. USA 82:3688 (1985); Hwang et al, Proc. Natl Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidyl ethanolamine (PEG-PE). Liposomes are extruded through filters of defined pore size to yield liposomes with the desired diameter. Fragments of the molecules of the present invention can be conjugated to the liposomes via a disulfide interchange reaction (Martin et al. J. Biol. Chem. 257:286-288 (1982). A chemotherapeutic agent is optionally contained within the liposome. See Gabizon et al. J. National Cancer Inst. 81(19)1484 (1989).

For the prevention or treatment of disease, the appropriate dosage of molecule will depend on the type of infection to be treated the severity and course of the disease, and whether the antibody is administered for preventive or therapeutic purposes. The molecule is suitably administered to the patient at one time or over a series of treatments. Depending on the type and severity of the disease, about 1 μg/kg to about 15 mg/kg of antibody is a typical initial candidate dosage for administration to the patient, whether, for example, by one or more separate administrations, or by continuous infusion.

Molecules containing a surrogate light chain of the present invention are suitable for use in the treatment or prevention of diseases. In one embodiment, the present invention provides a surrogate light chain-containing molecule for use as a medicament, or for the treatment of a disease. In another embodiment, the present invention provides the use of a surrogate light chain-containing molecule for the manufacture of a medicament for treating disease. The molecule may be a nucleic acid encoding an SLC polypeptide or SLC fusion.

In one aspect, the invention provides methods useful for treating a disease in a mammal, the methods including the step of administering a therapeutically effective amount of a surrogate light chain-containing molecule to the mammal. The therapeutic compositions can be administered short term (acute) or chronic, or intermittent as directed by physician.

The invention also provides kits and articles of manufacture containing materials useful for the treatment, prevention and/or diagnosis of disease. The kit includes a container and a label, which can be located on the container or associated with the container. The container may be a bottle, vial, syringe, or any other suitable container, and may be formed from various materials, such as glass or plastic. The container holds a composition having a surrogate light chain-containing molecule as described herein, and may have a sterile access port. Examples of containers include an intravenous solution bag or a vial with a stopper that can be pierced by a hypodeimic injection needle. The kits may have additional containers that hold various reagents, e.g., diluents and buffers. The label may provide a description of the composition as well as instructions for the intended use. Kits containing the molecules find use, e.g., for cellular assays, for purification or immunoprecipitation of a polypeptide from cells. For example, for isolation and purification of a protein, the kit can contain a surrogate light chain-containing molecule that binds the protein coupled to beads (e.g., sepharose beads). Kits can be provided which contain the molecules for detection and quantitation of the protein in vitro, e.g., in an ELISA or a Western blot. Such molecules useful for detection may be provided with a label such as a fluorescent or radiolabel.

The kit has at least one container that includes a molecule comprising a surrogate light chain described herein as the active agent. A label may be provided indicating that the composition may be used to treat a disease. The label may also provide instructions for administration to a subject in need of treatment. The kit may further contain an additional container having a pharmaceutically-acceptable buffer, such as bacteriostatic water for injection (BWFI), phosphate-buffered saline, Ringer's solution and dextrose solution. Finally, the kit may also contain any other suitable materials, including other buffers, diluents, filters, needles, and syringes.

Although in the foregoing description the invention is illustrated with reference to certain embodiments, it is not so limited. Indeed, various modifications of the invention in addition to those shown and described herein will become apparent to those skilled in the art from the foregoing description and fall within the scope of the appended claims.

All publications, patents and patent applications cited herein are hereby incorporated by reference in their entirety for all purposes to the same extent as if each individual publication, patent or patent application were specifically and individually indicated to be so incorporated by reference.

Further details of the invention are provided in the following non-limiting examples.

### EXAMPLES

### Example 1

### General Construction of SVD Surroglobulins and Binding Results

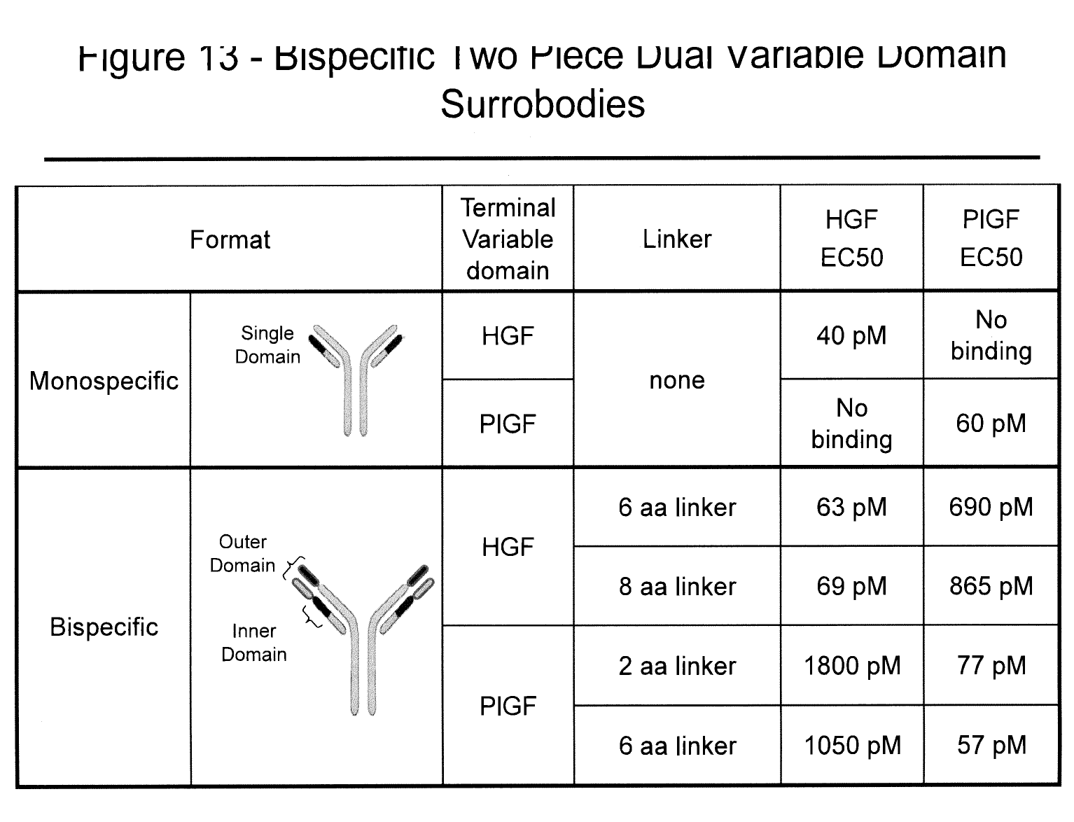

A stacked variable domain (SVD) Surroglobulin with binding specificities to both Hepatocyte Growth Factor (HGF) and Placenta Growth Factor (P1GF) was constructed. First, Surrobodies that specifically bind human forms of HGF or P1GF were discovered from a synthetic human surrobody library essentially as described (Xu et al., Proc. Natl. Acad. Sci. USA 2008, 105(31):10756-61). Specific binding clones to either HGF or P1GF were mammalian codon optimized and expressed transiently as bivalent surroglobulins in HEK293 cells. The respective surroglobulins were purified from the resulting supernatants via Protein A chromatography. Following a dialysis step to buffer exchange the proteins into PBS the resulting surroglobulins were quantitated by A280, analyzed by SDS-PAGE and size exclusion chromatography for purity and general protein disposition. Finally, these monospecific surroglobulins were each tested by target-based capture ELISA, as shown in FIG. 13. To create the first component of the SVD-SgG we recombinantly fused a fragment of the surrogate light chain (SLC) to the N-terminus of the anti-P1GF VH domain. In some instances we inserted an intervening 2 amino acid (Gly-Ala) or 8 amino acid (Gly-Gly-Gly-Ser-Gly-Gly-Gly-Ser (SEQ ID NO:111)) synthetic linker between the SLC fragment and the VH domain. To create the second component of the SVD-SgG we recombinantly fused the anti-HGF VH domain to the N-terminus of the surrogate light chain (VpreB/λ5 fusion protein). Again, in some instances we inserted an intervening 2 amino acid (Gly-Ala) or 8 amino acid (Gly-Gly-Gly-Ser-Gly-Gly-Gly-Ser (SEQ ID NO:111)) synthetic linker between the VH fragment and the N-terminus of the VpreB/λ5 fusion protein. The resulting heteromeric SVD-SgGs were produced by cotransfection of their respective component plasmids to produce bivalent SVD-SgGs in HEK293 cells, as described above. These SVD-SgGs were purified from the resulting supernatants via Protein A chromatography. Following a dialysis step to buffer exchange the proteins into PBS the resulting SVD-SgGs were quantitated by A280, analyzed by SDS-PAGE and size exclusion chromatography for purity and general protein disposition. Finally, the SVD-SgGs were each tested by target-based capture ELISA and the resulting data is shown in FIG. 13.

In addition to the SVD-SgGs described above we also made constructs that recombinantly fuse a fragment of the SLC to the N-terminus of the anti-HGF VH domain and one where the anti-P1GF VH domain is fused to the N-terminus of the surrogate light chain (SLC), both with similar fusion points and linker composition to that described above. These described constructs functionally reverse the orientation of the HGF and P1GF binding domains described above.

Results of binding analysis revealed that binding of target on the “outer” domain maintained near parental affinities, while binding of target to the “inner” domain typically were not as strong as parental affinities. In particular HGF binding affinities were more dramatically reduced when bound on the inner domain, compared to when P1GF was binding from the inner domain, as shown in FIG. 13.

### Example 2

### Combinatorially Expanded Linker Design

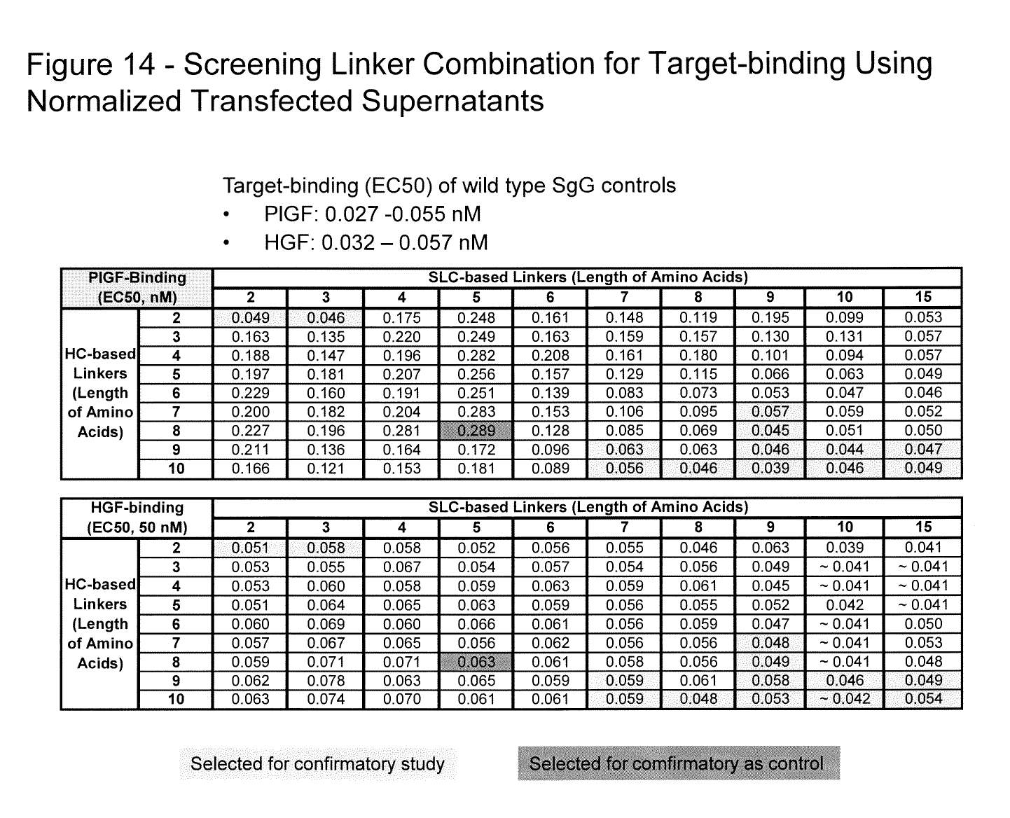

In Example 1, only “matched” linker lengths and fusion sites were used. In this example we tested a larger set of linker lengths, including all possible combinations 14. For this study only the anti-HGF “out”/anti-P1GF “in” orientation of stacked variable domains were produced for testing. Each linker and junction is as described in FIG. 14. Each construct was produced as described earlier, but this time protein quantitation was determined by quantitative anti-Fc biolayer interferometry (Octet, Fortebio) of transfected supernatants. Supernatants were normalized and diluted to produce ELISA-based binding isotherms specific to each of the targets and binding affinities determined by Prism analysis (Graphpad). The results of this work showed inner domain anti-P1GF binding varied within a one to ten-fold range from the parental SgG, while HGF binding only varied between one and three-fold range from the parental SgG. In this example the better P1GF binders tended to be longer lengths, though not exclusively.

### Example 3

### General Construction of Single-Chain Based-SVD Surroglobulins and Binding Results

An alternative to the format of the previously described stacked variable domain (SVD) Surroglobulin is shown in FIG. 1B. Specifically the first component of the complex is the single chain product of a heavy chain variable domain (VH) of the first surrobody linked to its cognate surrogate light chain that creates the “outer” binding domain, which is in turn linked to the surrogate light chain of a second surrobody. The second component of the SVD complex is the heavy chain variable domain (VH) of a second surrobody, as illustrated in FIG. 1B, which creates the “inner” binding domain. This second heavy chain is usually followed by the constant domain (CH1) and if desired the Fc region for avid binding to both each distinct specificity.

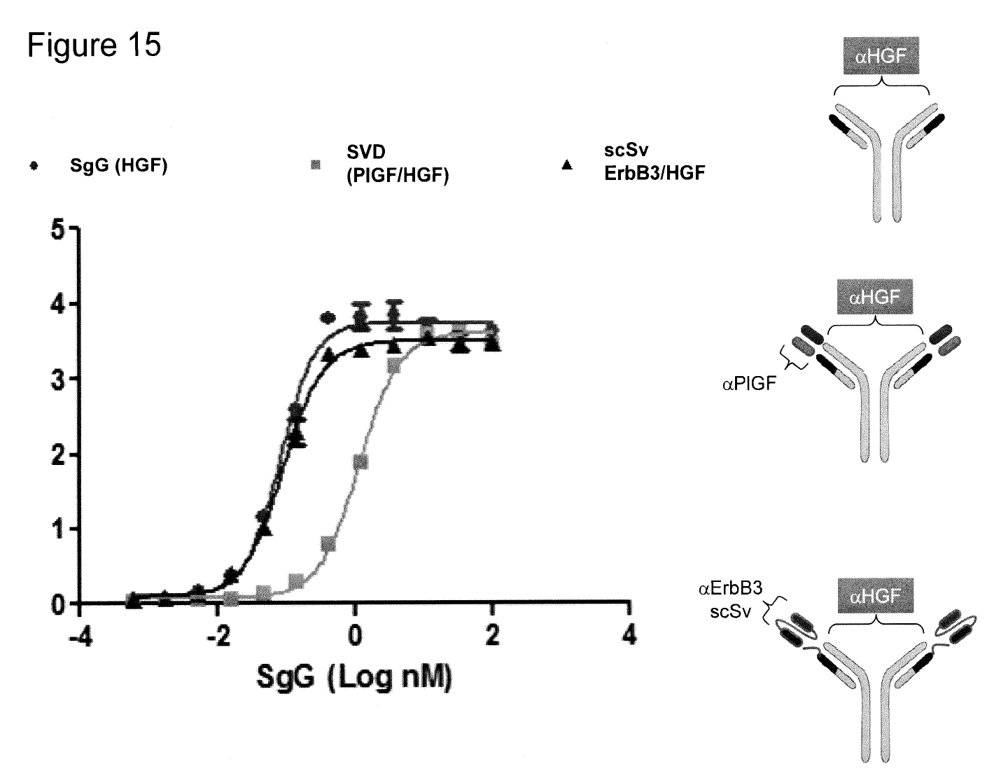

In a previous example a set of stacked variable domain Surroglobulin with binding specificities to both Hepatocyte Growth Factor (HGF) and Placental Growth Factor (P1GF) showed significant loss of binding to HGF, when the anti-HGF VH domain was fused to the C-terminus of VpreB as an “inner” binding domain. The loss in apparent binding affinity may be due to restricted access to the anti-HGF VH domain caused by the linker or the proximity of the Vpre domain. In either case, if the linker were not present the resulting SVD may display more native like binding affinity for HGF. To test this we recombinantly fused a single chain anti-P1GF Surrobody to the amino-terminus of the N-terminus of a fusion form of the surrogate light chain. This construct was co-transfected with the full length heavy chain of an anti-HGF surroglobulin, as described above. The resulting protein was purified, buffer exchanged, and analyzed as described above. The resulting protein was tested by ELISA for HGF binding and compared to both the parental monospecific anti-HGF surroglobulin and against the SVD-SgG construct described above and illustrated in FIG. 1A. As seen before the parental anti-HGF SgG bound with high affinity and the SVD-SgG bound with considerably lower affinity. However removal of the linked fusion to the anti-HGF Vh domain restored parental affinity (see Table 15.1 and FIG. 15: Bispecific SVD vs. scSv SgG: HGF-binding derived from heavy chain).

TABLE 15.1

| | |
|---|---|
| HC Detection | Key |

| SgG | EC50 | R² | FIG. 15 |
|---|---|---|---|
| SgG (HGF) | 0.080 | 0.9923 | circle |
| SgG (PlGF/HIGF SVD) | 1.118 | 0.9976 | square ▪ |
| SgG (ErbB3_scSv/HGF) | 0.088 | 0.9969 | triangle ▴ |

Note that in this example the first binding domain specificity is created as a single chain construct fused to the surrogate light chain of a second binding specificity to restore native binding affinities of a parental SgG. However, if the second binding domain maintained native binding affinities in the presence of a fusion on the N-terminus, then the single chain construct can be fused to obtain a similar effect to that described above. Furthermore it is possible to fuse distinct single chain binding domains to both the amino terminus of the surrogate light chain and the amino terminus of the heavy chain to create a trispecific, avid heteromeric binding protein.

### Example 4

### Constructing and Testing the Function of an SVD-SgG for Binding and Inhibition VEGF

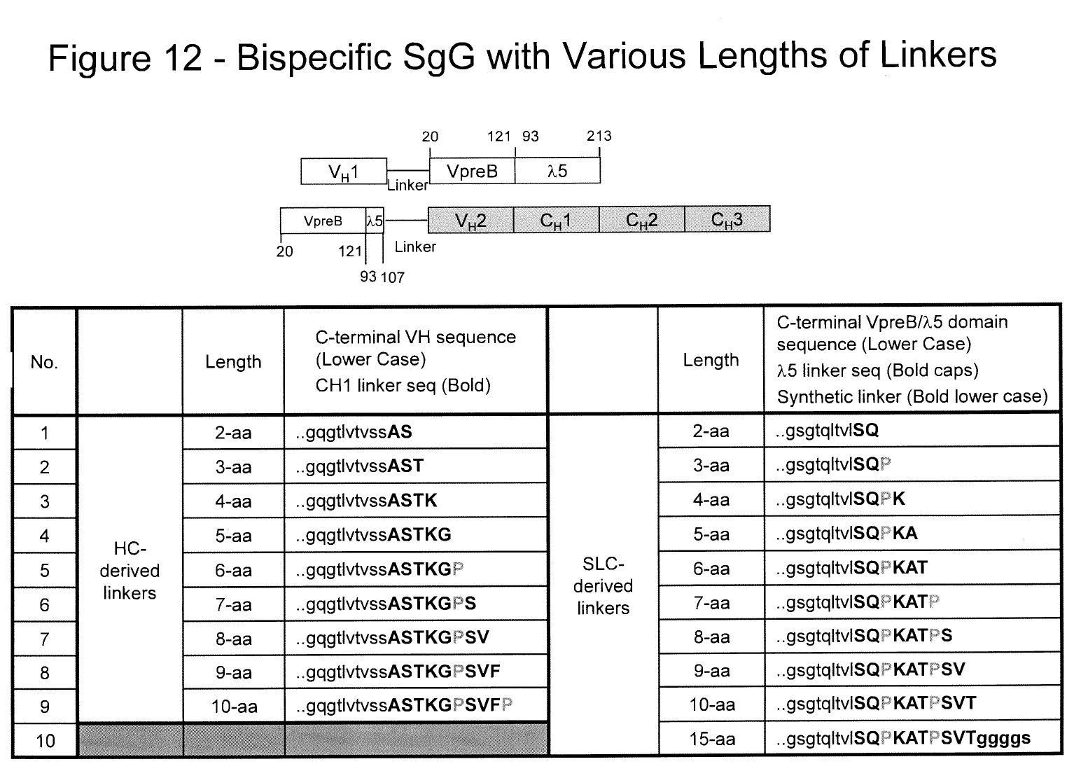

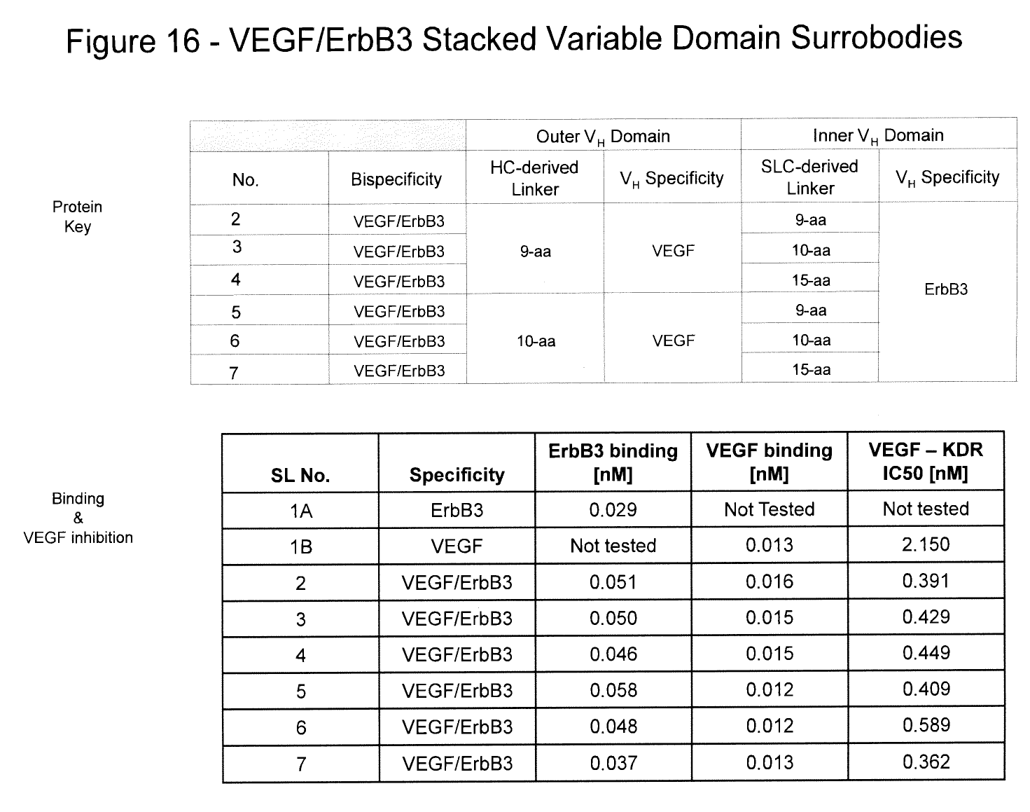

SVD-SgGs were designed and produced as described with an “outer” binding domain specificity based upon a previously identified VEGF neutralizing Surroglobulin and an “inner” domain specificity based upon a previously identified ErbB3 Surroboglobulin. Several linker combinations were generated, as listed in FIGS. 12 and 16. Binding analysis of the resulting panel of SVD-SgGs showed they bound with affinities that were not significantly different from the parental SgGs (FIG. 16). Remarkably, the VEGF neutralization IC50 values showed a significant measure of improvement compared to the parental SgG (360-590 pM compared to 2.1 nM) as shown in FIG. 16.

### Example 5

### Construction of Single Target SVD-SgGs

The previous examples described the construction of SVD-SgG using distinct VH domains specific to distinct targets. However it is well established that polyclonal antibodies are sometimes advantageous compared to monoclonal antibodies. In some instances cocktails or simple mixtures of antibodies raised against different epitopes, when combined, can exceed the efficacy of either monoclonal alone. For example EGFr activity can be inhibited to a greater extent by simultaneously engaging several extracellular epitopes with antibody mixtures. However, this poses a practical problem in insuring a consistent mixture and activity is preserved in a cocktail approach. To address this challenge, a panel of SVD-SgGs composed of combinations of Vh domains of neutralizing surroglobulins and combinatorial linker diversity is created to identify combinations with potentiated or additional activity. The beneficial combination generates a more potent agent, as well as a more consistent product than a cocktail admixture of biologics.

In another example of targeting a single molecule, single Vh is used for each of the four binding sites of an SVD-SgG, to create a molecule that is capable of either binding stoichiometrically larger amounts of target or creating higher order clusters of the targeted protein. One functionally distinct example involves the generation of an SVD-SgG containing only the Vh domain from a Death Receptor agonist surroglobulin. Death Receptors often need crosslinking to create higher order binding for activation. In this instance, molecules capable of crosslinking through a possible tetravalent interaction are created.

### Example 6

### Constructing of a Non-Avid Binding(“Monomeric”) SVD-SgG VEGF

In the previous examples, the SVD-SgGs were designed to maintain avid binding towards each respective specificity, however in the case of some targets avid binding is undesirable. For instance some growth factor receptors, when dimerized with bivalent antibodies causes unwanted activation, even though the corresponding monomeric Fabs are neutralizing. One such receptor is the HGF receptor, c-met and the T cell receptor, CD3. In this instance, Vh domains against both of these receptors are combined to create a structure similar to that shown in FIG. 18 that recruits T cells to c-met tumors to kill tumors, without inappropriately activating T cells or enhancing the proliferation of the c-met bearing tumors. Though fusion of the stacked variable domains would commonly be placed at the N-terminus of the Fc, the binding domains are fused to the Fc C-terminus. In any instance, either of these formats are combined with each other and with fat mats described in the examples herein.

To more efficiently produce this type of molecule, one uses an Fc construct that favors heterodimeric Fc production and stability to increase the productive yields of the desired construct.

### Example 7

### Constructing Mixed Valency SVD-SgGs

In the previous example the benefits of monomeric binding were leveraged through a combination. In this example a molecule that combines multiple specificities and valencies is created. In one possible scenario, it is beneficial to recruit T cells to c-met/ErbB3 sensitive tumors. As before, CD3 and c-met are engaged in a monomeric manner, while ErbB3 is more effectively engaged as a bivalent. One example of such a desired format is shown in FIG. 19. In this instance the monomeric specificities are harbored on opposing Fc fusions, while the ErbB3 bivalent specificity is harbored by Vh fusions to the amino-terminus of the surrogate light chain.

Another desirable mixed valency molecule is a bispecific molecule with an avid presentation for one specificity and a monovalent presentation to a second specificity. In this instance a monomeric binding domain similar to the inner domain of the previous non-avid binding example is combined with fully bivalent binding sites, essentially as diagrammed in FIG. 17 (right panel) In this instance, it is beneficial to utilize a c-met clone in the monovalent position and ErbB3 into the remaining bivalent slot. As shown in the FIG. 17 (right panel) the orientation of the bivalent binding components is linked to the Fc containing polypeptide or the non-Fc containing polypeptide.

### Example 8

### Single Chain SVD Surrobody Homodimeric Constructs

In a previous example we described the generation of single chain surrobodies and their fusion to Surrobglobulin polypeptides. In this instance, bispecific tandem single chain surrobodies is created as illustrated in FIG. 17 (left panel). These tandem single chain constructs are fused to an Fc to create a bivalent, homodimeric avid binder that has production conveniences over that of heterodimeric constructs.

### Example 9

### Construction and Testing of a Bispecific SVD-SgG

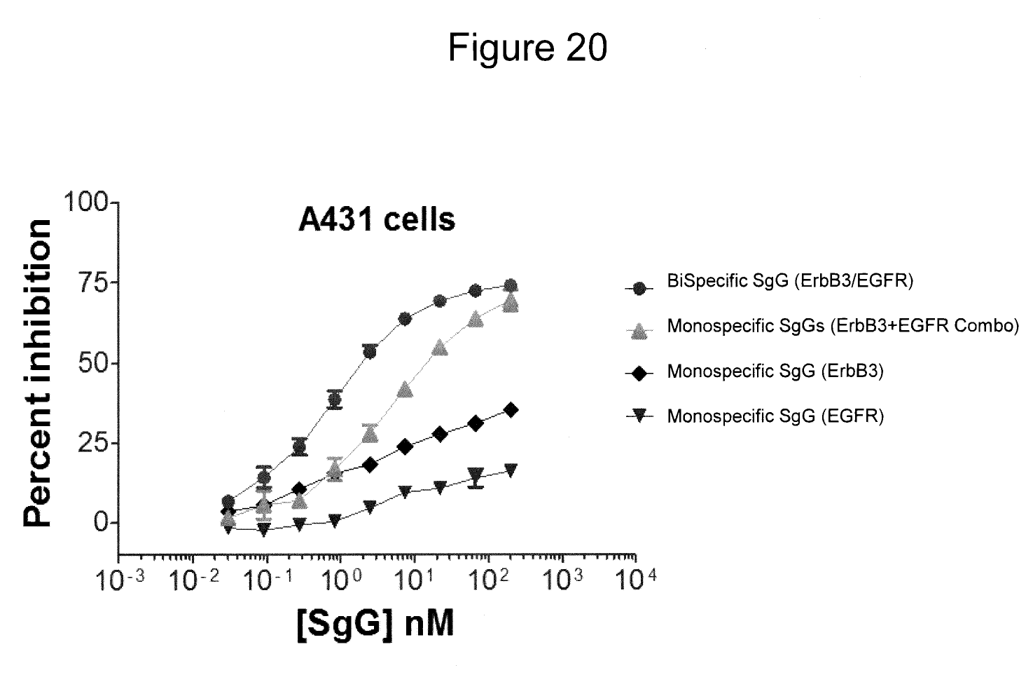

Stacked Variable Domain (SVD)-SgGs were designed and produced as described with an “outer” binding domain specificity based upon a previously identified EGFR neutralizing Surroglobulin (SgG) and an “inner” domain specificity based upon a previously identified ErbB3 Surroglobulin (SgG). This type of SVD-SgG format is illustrated in FIG. 1A. Binding to each of the individual targets were done individually in ELISA format. The resulting ELISA-based affinities for one such bispecific SgG was 0.124 nM for EGFR and 0.062 nM for ErbB3. Next, we tested the ability of the SVD-SgG to inhibit proliferation of A431 cells. Specifically, cells plated in 96 well plates were first incubated with the bispecific SgG and single specificity controls in serum-free media for 60 minutes, then NRG (human NRG1-β1 EGF domain, R&D Systems) was added to 10 ng/ml final concentration and the cells were then incubated for 4 days at 37 degrees C. in 5% CO2. Relative cell number due to proliferation was assessed through a luminescent substrate based assay (CellTiter-Glo, Promega). From this analysis, both combinations of EGFR and ErbB3 Surrobodies, either as a monospecific cocktail or a single molecule SVD-SgG demonstrated greater efficacy in proliferative inhibition, compared to either single monospecific agent. In terms of potency, the SVD-SgG demonstrated greater potency than the cocktail of monospecific agents with an IC₅₀ of 0.79 nM compared to an IC₅₀ of 5.2 nM respectively. (FIG. 20).

### Example 10

### SVD Surrobodies Bind VEGF

To determine the binding affinities of the anti-VEGF domains within the different SVD constructs we tested binding by ELISA-based assays. In brief rhVEGF165 (Peprotech #100-20) was coated overnight at 4 C onto ELISA plates at 100 ng/well. The next day, the plates were washed 3× with PBS-T (Tween 0.05%). Next wells were blocked with 0.2 ml/well of 1% BSA+PBS-T for 1 hr at room temperature and then the blocking solution was removed and dilutions of SgG/bispecifics in 1% BSA+PBS-T 0.1 ml/well are added to blocked wells and incubated for 1 hr at room temperature and then washed 3× with PBS-T. Detection was accomplished by incubating the wells with Donkey anti-hu Fc, HRP conjugated (Jackson #709-035-098) at 1:5000 dilution in 1% BSA+PBS-T for 1 hr at room temperature. Finally, plates were washed 6× with PBS-T and then developed by the addition of 0.1 ml/well TMB substrate (BioFx #TMB W-1000-01) for 2 min. The colorimetric reactions stopped by the addition of 0.1 ml/well low pH stop solution (BioFx #LSTP-1000-01) and A450 nm read and recorded. Affinities were determined after Prism (GraphPad) analysis.

As shown in Table 10.1 and Table 10.2 below, Angiopoietin/VEGF bispecific properties are similar to parental Surrobodies.

TABLE 10.1

| | All values [nM] | | | | |
|---|---|---|---|---|---|
| | Ang-1 | Ang-2 | | VEGF | |
| Molecule | Cell | ELISA | ELISA | ELISA | Cell |
| (specificity) | IC50 | Binding | Inhibition | Binding | IC50 |
| (Ang 1/2) | 0.281 | 0.020 | 0.156 | | |
| (VEGF) | | | | 0.086 | 0.139 |
| (Ang 1/2 x | ~0.3 | 0.023 | 0.173 | 0.055 | 0.464 |
| VEGF) | | | | | |
| Avid | | | | | |
| Bispecific | | | | | |

The Ang 1/2×VEGF bispecific SVD of Table 10.1 is made up of one polypeptide chain comprising an amino acid sequence shown as SEQ ID NO:152 and another polypeptide chain comprising an amino acid sequence shown as SEQ ID NO:154.

TABLE 10.2

| | | Target binding EC50 (nM) | | | IC50 | | | | |
|---|---|---|---|---|---|---|---|---|---|
| | | VEGF | | | (nM) | | | | |
| | | 100 ng | | | Ang- | Parental | | Parental | |
| | ELISA | coat | Ang-2 | Ang-1 | 2/Tie2 | outside | Linker | inside | Linker |
| Ang × | 2 | 0.05291 | 0.02178 | | 0.1645 | 18 | | 21 | 9 |
| VEGF | 3 | 0.04819 | 0.02168 | | 0.1641 | 18 | 9 | 21 | 10 |
| | 4 | 0.04385 | 0.02244 | | 0.1829 | 18 | | 21 | 15 |
| | 5 | 0.04968 | 0.01866 | | 0.1702 | 18 | | 21 | 9 |
| | 6 | 0.05513 | 0.02344 | 1.1 | 0.1734 | 18 | 10 | 21 | 10 |
| | 7 | 0.05092 | 0.02072 | | 0.1764 | 18 | | 21 | 15 |
| | 8 | 0.02451 | 0.02336 | | 0.1761 | 19 | 10 | 21 | 10 |
| VEGF × | 10 | 0.1225 | 0.09833 | | 0.577 | 20 | | 18 | 9 |
| Ang | 11 | 0.07039 | 0.03913 | | 0.6703 | 20 | 9 | 18 | 10 |
| | 12 | 0.1001 | 0.04555 | | 0.295 | 20 | | 18 | 15 |
| | 13 | 0.1587 | 0.1112 | | 0.5865 | 20 | | 18 | 9 |
| | 14 | 0.06804 | 0.05125 | | 0.4045 | 20 | 10 | 18 | 10 |
| | 15 | 0.0759 | 0.03848 | | 0.2118 | 20 | | 18 | 15 |
| | 16 | 0.02716 | 0.05044 | | 0.241 | 20 | 10 | 19 | 9 |
| | 17 | 0.03499 | 0.04682 | | 0.2713 | 20 | | 19 | 10 |

The SVD molecules listed in Table 10.2 are made up of two pairs of polypeptide chains, wherein each member of the first pair comprises an amino acid sequence shown as Polypeptide #1 below and each member of the second pair comprising an amino acid sequence shown as Polypeptide #2, as shown in Table 10.3 below.

TABLE 10.3

| Molecule | Polypeptide #1 | Polypeptide #2 |
|---|---|---|
| 2 | SEQ ID NO: 153 | SEQ ID NO: 156 |
| 3 | SEQ ID NO: 153 | SEQ ID NO: 154 |
| 4 | SEQ ID NO: 153 | SEQ ID NO: 155 |
| 5 | SEQ ID NO: 152 | SEQ ID NO: 156 |
| 6 | SEQ ID NO: 152 | SEQ ID NO: 154 |
| 7 | SEQ ID NO: 152 | SEQ ID NO: 155 |
| 8 | SEQ ID NO: 152 | SEQ ID NO: 154 |
| 10 | SEQ ID NO: 153 | SEQ ID NO: 158 |
| 11 | SEQ ID NO: 153 | SEQ ID NO: 157 |
| 12 | SEQ ID NO: 153 | SEQ ID NO: 159 |
| 13 | SEQ ID NO: 152 | SEQ ID NO: 158 |
| 14 | SEQ ID NO: 152 | SEQ ID NO: 157 |
| 15 | SEQ ID NO: 152 | SEQ ID NO: 159 |
| 16 | SEQ ID NO: 152 | SEQ ID NO: 201 |
| 17 | SEQ ID NO: 152 | SEQ ID NO: 202 |
| 18 | SEQ ID NO: 160 | SEQ ID NO: HC1 |
| 19 | SEQ ID NO: 160 | SEQ ID NO: HC2 |
| 20 | SEQ ID NO: 160 | SEQ ID NO: HC3 |
| 21 | SEQ ID NO: 160 | SEQ ID NO: HC4 |

### Example 11

### SVD Surrobodies Inhibit VEGF Stimulated HUVEC-2 Proliferation

To determine the inhibitory capacity of the SVD bispecifics we tested their ability to inhibit VEGF stimulated proliferation of HUVEC cells. Briefly, HUVEC-2 cells (BD #354151) were grown in EGM-2MV Microvascular endothelial cell growth medium-2 with growth factors (Lonza #CC-3202), trypsinized, and washed 3× with medium-199 (Lonza #12-117F) with 10% FBS. Cells were plated in 0.1 ml/well in M-199+10% FBS at 2E+04 cells/mL onto 96-well TC white Greiner plates (E&K #EK-25083) pre-coated with 0.1 ml/well 1% gelatin (Stem Cell #07903) for 15 min at room temperature. Cells were starved overnight at 37 C, 5% CO2. Dilutions of test article in M-199+10% FBS+3 ng/mL final concentration rhVEGF165 (Peprotech #100-20) or rmVEGF165 (PeproTech #450-32). Plates were incubated for 72 hrs at 37 C, 5% CO2. 0.1 ml/well were removed from wells and plates were allowed to equilibrate to room temperature for 30 min. 0.1 ml/well Cell Titer glo reagent (Promega #G7570) was added, plates then shaken for 2 min on shaker platform and incubated for 10 min at room temperature in the dark to equilibrate. Luminescence 0.1 sec setting is read using the Victor plate reader. Data was captured and then graphed and in some instances analyzed using Prism (GraphPad) analysis to determine IC50 values. Results are shown in Table 10.2 above and Table 11.1 below.

TABLE 11.1

| | | Outer domain | | Inner domain | |
|---|---|---|---|---|---|
| SVDs | | Parental | Linker | Parental | Linker |
| VEGF/Ang-2 | (1) | VEGF (3) | 10-aa | Ang-2 (4) | 9-aa |
| VEGF/ErbB3 | (2) | | | ErbB3 (5) | 10-aa |

The VEGF/Ang-2 bispecific SVD ((1) of Table 11.1) is made up of a pair of polypeptide chains, wherein each member of the first pair comprises an amino acid sequence shown as SEQ ID NO:152 and each member of the second pair comprises an amino acid sequence shown as SEQ ID NO:158. The VEGF/ErbB3 bispecific SVD ((2) of Table 11.1) is made up of a pair of polypeptide chains, wherein each member of the first pair comprises an amino acid sequence shown as SEQ ID NO:157 and each member of the second pair comprises an amino acid sequence shown as SEQ ID NO:158.

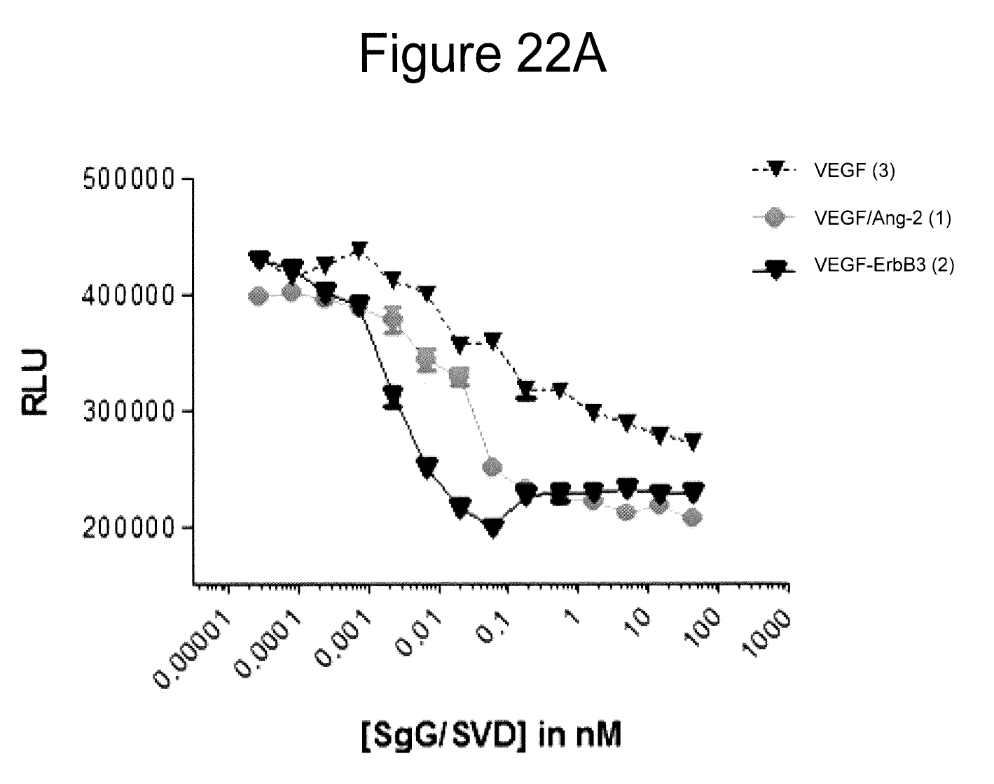

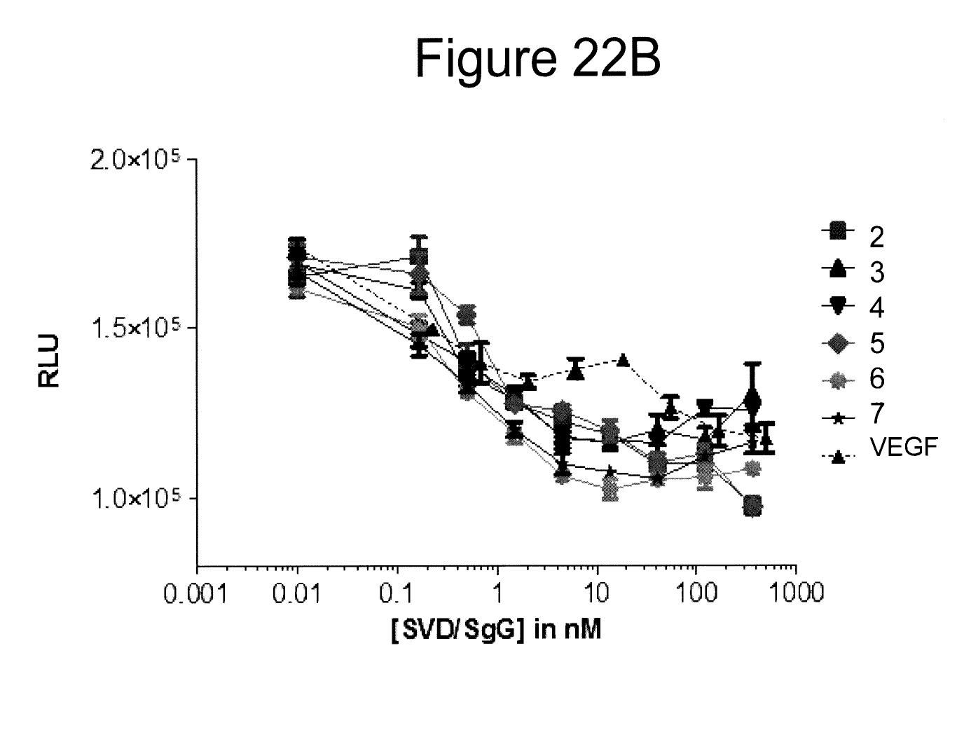

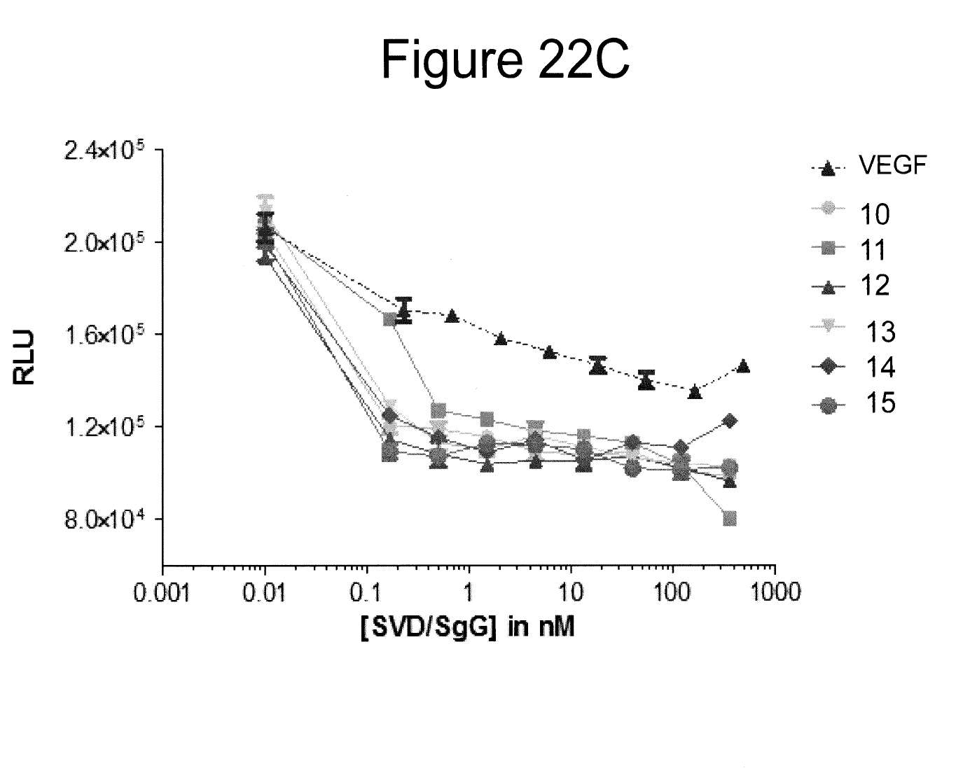

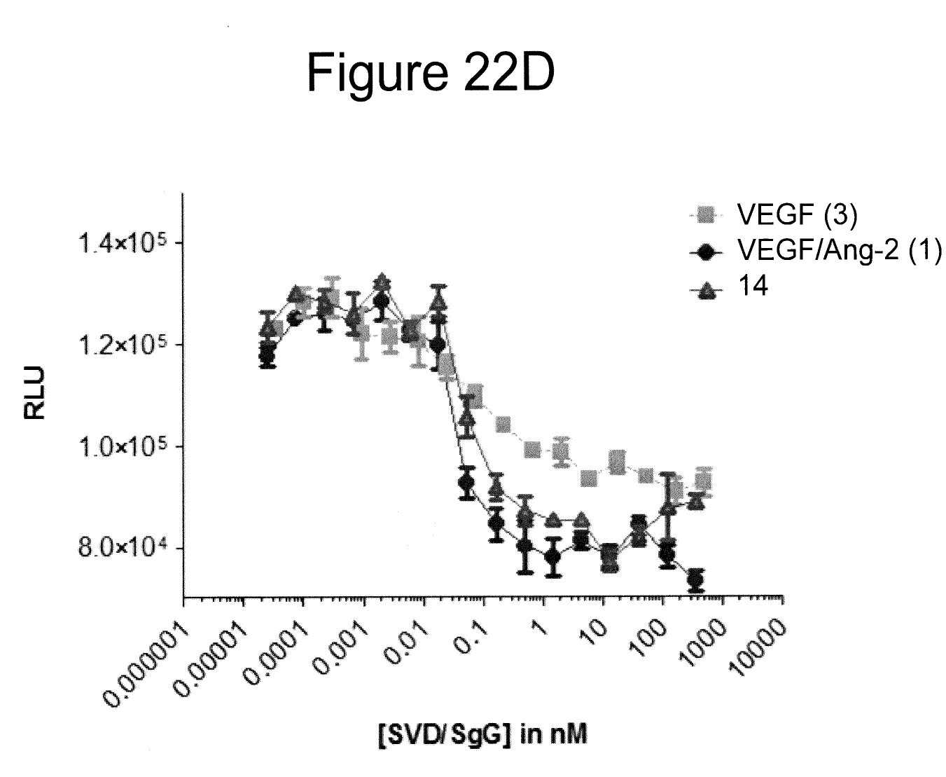

FIG. 22A-D demonstrate that SVD Surrobodies inhibit VEGF-stimulated HUVEC proliferation better than parental VEGF Surrobody. In FIG. 22A: VEGF (3) from Table 11.1 is the parental VEGF Surrobody; VEGF/Ang-2 (1) and VEGF-ErbB3 (2) are from Table 11.1. In FIG. 22B: VEGF is the parental VEGF Surrobody; 2 from Table 10.2; 3 from Table 10.2; 4 from Table 10.2; 5 from Table 10.2; 6 from Table 10.2; and 7 from Table 10.2. In FIG. 22C: VEGF is the parental VEGF Surrobody; 10 from Table 10.2; 11 from Table 10.2; 12 from Table 10.2; 13 from Table 10.2; 14 from Table 10.2; and 15 from Table 10.2. In FIG. 22D: VEGF (3) from Table 11.1 is the parental VEGF Surrobody; VEGF/Ang-2 (1) from Table 11.1; and 14 from Table 10.2.

### Example 12

### SVD Surrobodies Bind Both Angiopoietin-1 and Angiopoietin-2

To determine the binding affinities of the anti-Angiopoietin domains within the different SVD constructs we tested binding by ELISA-based assays. In brief Peprotech rhAng-1 (R&D #923-AN/CF) or rhAng-2 (R&D #623-AN/CF) were coated overnight at 4 C onto ELISA plates at 10 ng/well. Plates are washed 3× with PBS-T. Wells are blocked with 0.2 ml/well of 1% BSA+PBS-T for 1 hr at room temperature. Blocking solution was removed and dilutions of SgG/bispecifics in 1° ABSA-PPBS-T 0.1 ml/well were added to blocked wells and incubated for 1 hr at room temperature. Plates were washed 3× with PBS-T. Detection was accomplished by using Donkey anti-hu Fc, HRP conjugated (Jackson #709-035-098) diluted 1:5000 in 1% BSA+PBS-T for 1 hr at room temperature.

Plates were then washed 6× with PBS-T and developed with 0.1 ml/well TMB substrate (BioFx #TMB W-1000-01) for 2 min, reaction stopped with stop solution (BioFx #LSTP-1000-01) and read A450 nm. Data was captured and then graphed and in some instances analyzed using Prism (GraphPad) analysis to determine binding affinities. Results are shown in Table 10.1 and Table 10.2 above.

### Example 13

### SVD Surrobodies Inhibit Angiopoietin-2 Binding to Tie2

To determine the inhibitory capacity of the SVD bispecifics we tested their ability to inhibit Ang-2 binding to its cognate receptor Tie-2 in an ELISA-based binding assay. Briefly, recombinant Tie-2 protein (BD rhTie-2 (R&D #313-TI) was coated overnight at 4 C onto ELISA plates at 100 ng/well. Plates were washed 3× with PBS-T. Wells were blocked with 0.2 ml/well of 1% BSA+PBS-T for 1 hr at room temperature. Blocking solution was removed and dilutions of SgG/bispecifics in 1% BSA+PBS-T premixed for 30 min at RT with 125 ng/mL biotinylated rhAng-2 (R&D #BT623), 0.1 ml/well was added to blocked wells and incubated for 1 hr at room temperature. Plates were washed 3× with PBS-T. Detection was then accomplished by using streptavidin HRP diluted 1:5000 in 1% BSA+PBS-T for 1 hr at room temperature. Plates were then washed 6× with PBS-T and then developed with 0.1 ml/well TMB substrate (BioFx #TMB W-1000-01) for 2 min, reaction stopped with stop solution (BioFx #LSTP-1000-01) and read A450 nm. Data was captured and then graphed and in some instances analyzed using Prism (GraphPad) analysis to determine IC50 values. Results are shown in Table 10.1 and Table 10.2 above.

### Example 14

### Polypeptide Cross Complemented SVD Surroglobulins

A stacked variable domain (SVD) Surroglobulin is a heteromeric binding protein designed such that two domains from two different parental Surrobodies are covalently linked via a designed linker. Specifically the first binding component of the complex is the product of a heavy chain variable domain (VH) and a surrogate light chain domain from a second polypeptide. The second binding domain is the product of a heavy chain variable domain on the second polypeptide and a surrogate light chain domain from the first polypeptide.

FIG. 21 provides an example of a cross complemented SVD. In most cases one or both of the polypeptides will be conjugated to an immunoglobulin Fc protein, but won't necessarily require Fc fusion. Alternatively the crosscomplemented SVD could be used without an Fc, or it could be fused to another heterologous fusion partner such as Human Serum Albumin to impart better PK properties and avoid effector function.

### Example 15

### Cross Complemented VEGF x Angiopoietin SVD Surroglobulins

Cross complemented SVDs can be assembled from existing surrobody variable domains. In this instance we assemble molecules based upon combinations of anti-VEGF N-terminal variable domain conjugates to the Fc that are complemented with anti-angiopoietin N-terminal variable domain conjugates to the λ5 protein. Specifically, the VEGF variable heavy domain has an intervening portion of VpreB just upstream of the constant heavy domains, with differing linker lengths between the variable heavy domain and VpreB (FIG. 11A-C; SEQ ID NOS: 57-65). To produce a bispecific any of these previously described proteins need to be complemented with a complementary polypeptide construct. Such a complementary construct would be one that contains an anti-angiopoietin N-terminal variable domain, fused to the N-terminus of VpreB that is conjugated to λ5, such as SEQ ID NO: 152 or 153. Conversely, the opposing orientation could be produced by combining any of the Angiopoietin constructs (FIG. 11A-C; SEQ ID NOS: 57-65) (with the VEGF variable heavy domain fused to the amino terminus of VpreB, conjugated to λ5. Each of the resulting Bispecific SVDs can be tested for binding and biological activity as described previously. Previously described alternate formats, including linker variant constructs can also be readily assembled.

### Example 16

### SVD Surrobodies Inhibit Neuregulin Stimulated BxPC-3 Proliferation

To determine the inhibitory capacity of the VEGF x ErbB3 SVD bispecifics they were tested for their ability to inhibit Neuregulin (NRG) stimulated proliferation of BxPC-3 cells. Briefly, BxPC-3 cells were plated at a density of 10,000 cells/well in 96 well plates in serum-free medium. They were then treated with the indicated concentrations of SgGs for 30 minutes at 37° C. Next NRG1β was then added to a final concentration of 10 ng/ml and the cells were then allowed to grow for 96 hours. Following the growth period cell content was measured using Cell Titer-Glo® (Promega). Resulting data was captured and then graphed using Prism (GraphPad) analysis.

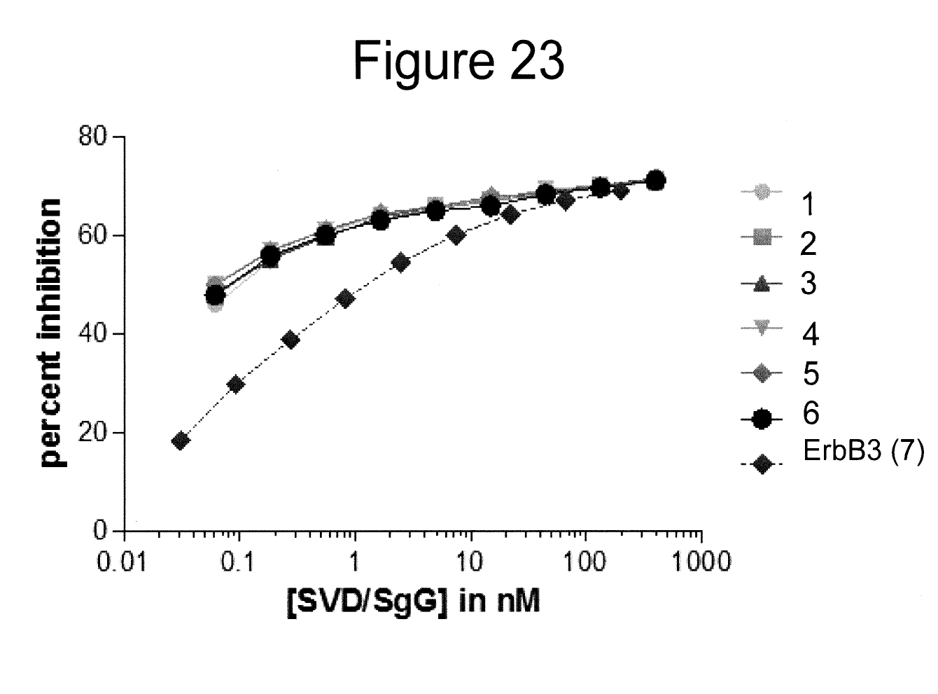

Table 15.1 below and FIG. 23 demonstrate SVD Surrobodies inhibit neuregulin-stimulated BxPC-3 proliferation better than parental ErbB3 Surrobody.

TABLE 15.1

| | | Outer domain | | Inner domain | |
|---|---|---|---|---|---|
| SVDs | | Parental | Linker | Parental | Linker |
| ErbB3/VEGF | 1 | ErbB3 (7) | 9-aa | VEGF (8) | 9-aa |
| | 2 | | 9-aa | | 10-aa |
| | 3 | | 9-aa | | 15-aa |
| | 4 | | 10-aa | | 9-aa |
| | 5 | | 10-aa | | 10-aa |
| | 6 | | 10-aa | | 15-aa |

In FIG. 23, ErbB3 (7) of Table 15.1 is the parental Surrobody; 1 from Table 15.1; 2 from Table 15.1; 3 from Table 15.1; 4 from Table 15.1; 5 from Table 15.1; and 6 from Table 15.1. The SVD molecules listed in Table 15.2 are made up of two pairs of polypeptide chains, wherein each member of the first pair comprises an amino acid sequence shown as Polypeptide #1 below and and each member of the second pair comprising an amino acid sequence shown as Polypeptide #2, as shown in Table 15.3 below.

TABLE 15.3

| Molecule | Polypeptide #1 | Polypeptide #2 |
|---|---|---|
| 1 | SEQ ID NO: 153 | SEQ ID NO: 156 |
| 2 | SEQ ID NO: 153 | SEQ ID NO: 154 |
| 3 | SEQ ID NO: 153 | SEQ ID NO: 155 |
| 4 | SEQ ID NO: 152 | SEQ ID NO: 156 |
| 5 | SEQ ID NO: 152 | SEQ ID NO: 154 |
| 6 | SEQ ID NO: 152 | SEQ ID NO: 155 |

1. A multi-specific Stacked Variable Domain (SVD) binding protein comprising a tandem product of a first heavy chain variable domain sequence conjugated to a second surrogate light chain sequence, associated with a first surrogate light chain sequence conjugated to a second heavy chain variable domain sequence, wherein the tandem product comprises a first binding domain and a second binding domain, wherein each of said first and second binding domains is formed by a surrogate light chain sequence and an antibody variable domain sequence, and wherein each of said first and second binding domains binds specifically to a different binding target.

2. The multi-specific SVD binding protein of claim 1, wherein said first and said second binding domain are present in a single polypeptide chain.

3. The multi-specific SVD binding protein of claim 1, wherein said first and said second binding domain are present on more than one polypeptide chain.

4. The multi-specific SVD binding protein of claim 1, wherein the C-terminus of said first heavy chain variable domain sequence is conjugated to the N-terminus of said second surrogate light chain sequence.

5. The multi-specific SVD binding protein of claim 1, wherein the C-terminus of said first surrogate light chain sequence is conjugated to the N-terminus of said second heavy chain variable domain sequence.

6. The multi-specific SVD binding protein of claim 1, wherein said first heavy chain variable domain sequence and said first surrogate light chain sequence together form a first binding domain specifically binding to a first target.

7. The multi-specific SVD binding protein of claim 1, wherein said second surrogate light chain sequence and said second heavy chain variable domain sequence together form a second binding domain specifically binding to a second target.

8. The multi-specific SVD binding protein of claim 1, wherein said first and said second surrogate light chain sequences are identical.

9. The multi-specific SVD binding protein of claim 1, wherein said first and said second surrogate light chain sequences are different.

10. The multi-specific SVD binding protein of claim 1 wherein said first and said second surrogate light chain sequences comprise a VpreB sequence.

11. The multi-specific SVD binding protein of claim 10 wherein said second surrogate light chain sequence further comprises a λ5 sequence.

12. The multi-specific SVD binding protein of claim 1, wherein said second heavy chain variable domain sequence further comprises a heavy chain constant domain sequence.

13. The multi-specific SVD binding protein of claim 12, wherein said second heavy chain variable domain sequence further comprises a CH1 sequence.

14. The multi-specific SVD binding protein of claim 12, wherein said second heavy chain variable domain sequence further comprises an Fc region.

15. (canceled)

16. The multi-specific SVD binding protein of claim 1, wherein the conjugation is by a linker sequence.

17. (canceled)

18. The multi-specific SVD binding protein of claim 1, wherein the conjugation is direct fusion.

19. The multi-specific SVD binding protein of claim 16, wherein the linker sequence comprises a sequence selected from the group consisting of: an antibody J region sequence, a λ5 sequence, a λ light chain constant region sequence, a κ light chain constant region sequence, synthetic sequence, and any combination thereof.

20. The multi-specific SVD binding protein of claim 19, wherein the synthetic sequence is (Gly-Gly-Gly-Ser)ₙ (SEQ ID NO: 109), (Gly-Gly-Gly-Gly-Ser)ₙ (SEQ ID NO: 110), or Gly-Ala, wherein n is at least 1.

21. The multi-specific SVD binding protein of claim 18, wherein the C-terminus of the first heavy chain variable domain sequence is fused to the N-terminus of the second surrogate light chain sequence forming a first polypeptide chain.

22. The multi-specific SVD binding protein of claim 18, wherein the C-terminus of the second surrogate light chain sequence is fused to the N-terminus of the second heavy chain variable domain sequence forming a second polypeptide chain.

23. The multi-specific SVD binding protein of claim 21 or 22, wherein a binding site to a target is formed between a surrogate light chain sequence and a heavy chain variable domain sequence on different polypeptide chains.

24. The multi-specific SVD binding protein of claim 21 or 22, wherein a binding site to a target is formed between a surrogate light chain sequence and a heavy chain variable domain sequence on the same polypeptide chains.

25. (canceled)

26. A first polypeptide chain comprising an antibody heavy chain variable region sequence, specific for a first target, C-terminally conjugated to a polypeptide sequence comprising a VpreB sequence.

27. The polypeptide chain of claim 26 associated with a second polypeptide chain comprising a VpreB sequence, conjugated to the N-terminus of an antibody heavy chain comprising a variable region sequence specific for a second target.

28. The polypeptide chain of claim 27, wherein the antibody heavy chain variable region sequence of the first polypeptide chain and the VpreB sequence of the second polypeptide chain form a binding site for said first target.

29. A heteromeric bispecific binding protein comprising the first polypeptide chain of claim 26, associated with the second polypeptide of claim 27.

30. The heteromeric bispecific binding protein of claim 29, wherein the heavy chain variable region of the second antibody heavy chain variable region sequence specific for said second target and the VpreB sequence of the first polypeptide chain form a binding site for a second target.

31. A heteromeric bispecific binding protein comprising one pair of the first polypeptide chain of claim 1 and one pair of the second polypeptide chain of claim 2.

32. The heteromeric bispecific binding protein of claim 31, wherein the heavy chain variable region of the second antibody heavy chain variable region sequence specific for said second target and the VpreB sequence of the first polypeptide chain form a binding site for a second target.

33-35. (canceled)

36. The polypeptide chain of claim 26 or 27 or the heteromeric bispecific binding protein of claim 4 or 5, wherein in the second polypeptide chain the conjugation is by a linker sequence.

37-38. (canceled)

39. The polypeptide chain of claim 36, wherein the linker sequence between the antibody heavy chain variable region sequence and the VpreB sequence of the first polypeptide chain comprises a sequence selected from the group consisting of: an antibody J region sequence, an antibody constant domain region sequence, a synthetic sequence, and any combination thereof.

40. The polypeptide chain of claim 36, wherein the linker sequence between the antibody heavy chain variable region sequence and the VpreB sequence of the first polypeptide chain comprises a sequence selected from the group consisting of:
(SEQ ID NO:)
Xaa_{g} Ala Ser Xaaₕ,
(SEQ ID NO:)
Xaa_{g} Ala Ser Thr Xaaₕ,
(SEQ ID NO:)
Xaa_{g} Ala Ser Thr Lys Xaaₕ,
(SEQ ID NO:)
Xaa_{g} Ala Ser Thr Lys Gly Xaaₕ,
(SEQ ID NO:)
Xaa_{g} Ala Ser Thr Lys Gly Pro Xaaₕ,
(SEQ ID NO:)
Xaa_{g} Ala Ser Thr Lys Gly Pro Ser Xaaₕ,
(SEQ ID NO:)
Xaa_{g} Ala Ser Thr Lys Gly Pro Ser Val Xaaₕ,
(SEQ ID NO:)
Xaa_{g} Ala Ser Thr Lys Gly Pro Ser Val Phe Xaaₕ,
and
(SEQ ID NO:)
Xaa_{g} Ala Ser Thr Lys Gly Pro Ser Val Phe Pro Xaaₕ,
wherein the Xaa is any amino acid, g is 0 to 10 amino acids, and h is 0 to 10 amino acids.

41. The polypeptide chain of claim 40, wherein Xaa_{g} comprises a sequence selected from the group consisting of
Ser,
Ser Ser,
Val Ser Ser,
(SEQ ID NO:)
Thr Val Ser Ser,
(SEQ ID NO:)
Val Thr Val Ser Ser,
(SEQ ID NO:)
Leu Val Thr Val Ser Ser,
(SEQ ID NO:)
Thr Leu Val Thr Val Ser Ser,
(SEQ ID NO:)
Gly Thr Leu Val Thr Val Ser Ser,
(SEQ ID NO:)
Gln Gly Thr Leu Val Thr Val Ser Ser,
and
(SEQ ID NO:)
Gly Gln Gly Thr Leu Val Thr Val Ser Ser.

42. The polypeptide chain of claim 40, wherein Xaaₕ comprises a sequence selected from the group consisting of
Gln,
Gln Pro,
Gln Pro Val,
Gln Pro Val Leu,
(SEQ ID NO)
Gln Pro Val Leu His,
(SEQ ID NO:)
Gln Pro Val Leu His Gln,
(SEQ ID NO:)
Gln Pro Val Leu His Gln Pro,
(SEQ ID NO:)
Gln Pro Val Leu His Gln Pro Pro,
(SEQ ID NO:)
Gln Pro Val Leu His Gln Pro Pro Ala,
and
(SEQ ID NO:)
Gln Pro Val Leu His Gln Pro Pro Ala Met.

43. The polypeptide chain of claim 36, wherein the linker sequence between the antibody heavy chain variable region sequence and the VpreB sequence of the second polypeptide chain comprises a sequence selected from the group consisting of: a λ5 sequence, an antibody J region sequence, a λ light chain constant region sequence, a κ light chain constant region sequence, a synthetic sequence, and any combination thereof.

44. The polypeptide chain of claim 36, wherein the linker sequence between the antibody heavy chain variable region sequence and the VpreB sequence of the second polypeptide chain comprises a sequence selected from the group consisting of:
(SEQ ID NO:)
Xaaⱼ Ser Gln Xaaₖ,
(SEQ ID NO:)
Xaaⱼ Ser Gln Pro Xaaₖ,
(SEQ ID NO:)
Xaaⱼ Ser Gln Pro Lys Xaaₖ,
(SEQ ID NO:)
Xaaⱼ Ser Gln Pro Lys Ala Xaaₖ,
(SEQ ID NO:)
Xaaⱼ Ser Gln Pro Lys Ala Thr Xaaₖ,
(SEQ ID NO:)
Xaaⱼ Ser Gln Pro Lys Ala Thr Pro Xaaₖ,
(SEQ ID NO:)
Xaaⱼ Ser Gln Pro Lys Ala Thr Pro Ser Xaaₖ,
(SEQ ID NO:)
Xaaⱼ Ser Gln Pro Lys Ala Thr Pro Ser Val Xaaₖ,
(SEQ ID NO:)
Xaaⱼ Ser Gln Pro Lys Ala Thr Pro Ser Val Thr Xaaₖ,
and
(SEQ ID NO:)
Xaaⱼ Ser Gln Pro Lys Ala Thr Pro Ser Val Thr Gly
Gly Gly Gly Ser Xaaₖ,
wherein Xaa is any amino acid, j is 0 to 10 amino acids, and k is 0 to 6 amino acids.

45. The polypeptide chain of claim 44, wherein Xaaⱼ comprises a sequence selected from the group consisting of

| | |
|---|---|
| Leu, | |
| Val Leu, | |
| Thr Val Leu, | |
| Leu Thr Val Leu, | (SEQ ID NO:) |
| Gln Leu Thr Val Leu, | (SEQ ID NO:) |
| Thr Gln Leu Thr Val Leu, | (SEQ ID NO:) |
| Gly Thr Gln Leu Thr Val Leu, | (SEQ ID NO:) |
| Ser Gly Thr Gln Leu Thr Val Leu, | (SEQ ID NO:) |
| and | |
| Gly Ser Gly Thr Gln Leu Thr Val Leu. | (SEQ ID NO:) |

46. The polypeptide chain of claim 44, wherein Xaaₖ comprises a sequence selected from the group consisting of

| | |
|---|---|
| Gln, | |
| Gln Val, | |
| Gln Val Gln, | |
| Gln Val Gln Leu, | (SEQ ID NO:) |
| Gln Val Gln Leu Val, | (SEQ ID NO:) |
| and | |
| Gln Val Gln Leu Val Gln. | (SEQ ID NO:) |

47. (canceled)

48. The polypeptide chain of claim 26 or 27, or the heteromeric bispecific binding protein of claim 4 or 5, wherein the VpreB sequence is fused, at its C-terminus, to a heterologous sequence.

49. The polypeptide chain or the heteromeric bispecific binding protein of claim 48, wherein the heterogenous sequence is selected from the group consisting of a λ5 sequence, an antibody J-region sequence, and a light chain constant domain region sequence.

50. A first polypeptide chain comprising an antibody heavy chain variable region sequence specific for a first target, C-terminally conjugated to a first polypeptide sequence comprising a first VpreB sequence, wherein the first polypeptide sequence comprising the VpreB sequence is C-terminally conjugated to a second polypeptide sequence comprising a second VpreB sequence, conjugated to a heterologous sequence.

51. The polypeptide chain of claim 50 associated with a second polypeptide chain comprising an antibody heavy chain comprising a variable region sequence specific for a second polypeptide target.

52. The polypeptide chain of claim 50, wherein the antibody heavy chain variable region sequence of the first polypeptide chain and the first VpreB sequence of the first polypeptide chain form a binding site for said first target.

53. A heteromeric bispecific binding protein comprising one pair of the first polypeptide chain of claim 50 and one pair of the second polypeptide chain of claim 51.

54. The heteromeric bispecific binding protein of claim 53, wherein the heavy chain variable region of the second antibody heavy chain variable region sequence specific for said second target and the second VpreB sequence of the first polypeptide chain form a binding site for a second target.

55. The polypeptide chain of claim 50 or 51 or the heteromeric bispecific binding protein of claim 52 or 53, wherein in the first polypeptide chain the conjugation is by a linker sequence.

56-57. (canceled)

58. The polypeptide chain of claim 55, wherein the linker sequence between the antibody heavy chain variable region sequence and the first polypeptide sequence comprising a first VpreB sequence comprises the amino acid sequence Gly-Gly-Gly-Gly-Ser (SEQ ID NO: 108).

59. The polypeptide chain of claim 55, wherein the linker sequence between the first polypeptide sequence comprising a first VpreB sequence and the second polypeptide sequence comprising a second VpreB sequence comprises the amino acid sequence Gly-Ala.

60-62. (canceled)

63. A polypeptide chain comprising an antibody heavy chain variable region sequence specific for a first target, C-terminally conjugated to a first polypeptide sequence comprising a first surrogate light chain (SLC) sequence, wherein the first SLC sequence is C-terminally conjugated to an antibody heavy chain variable region sequence specific for a second target.

64-75. (canceled)

76. Original) A first polypeptide chain comprising an antibody heavy chain variable region sequence specific for a first target conjugated to a first polypeptide sequence comprising a first VpreB sequence, wherein the first polypeptide sequence comprising the first VpreB sequence is C-terminally conjugated to a second polypeptide sequence comprising a dimerization domain.

77. The polypeptide chain of claim 76 associated with a second polypeptide chain comprising a first polypeptide sequence that comprises a second VpreB sequence, wherein the first polypeptide sequence comprising the second VpreB sequence is C-terminally conjugated to an antibody heavy chain variable region sequence specific for a second target.

78-81. (canceled)

82. A heteromeric bispecific binding protein comprising the first and second polypeptide chains of claim 77, associated with each other.

83. The heteromeric bispecific binding protein of claim 82, wherein the heavy chain variable region sequence specific for said second target of the second polypeptide and the first VpreB sequence of the first polypeptide chain form a binding site for a second target.

84. The polypeptide chain of claim 76 or heteromeric bispecific binding protein of claim 83, wherein the conjugation is by a linker sequence.

85-86. (canceled)

87. The polypeptide chain or heteromultimeric bispecific binding protein of claim 84, wherein the linker sequence comprises a sequence selected from the group consisting of: an antibody J region sequence, a λ5 sequence, a λ light chain constant region sequence, a κ light chain constant region sequence, synthetic sequence, and any combination thereof.

88. (canceled)

89. The polypeptide chain of claim 76 or 77, or the heteromeric bispecific binding protein of claim 82 or 83, wherein the VpreB sequence is fused, at its C-terminus, to a heterologous sequence.

90. The polypeptide chain or the heteromeric bispecific binding protein of claim 89, wherein the heterogenous sequence is selected from the group consisting of a λ5 sequence and a light chain constant domain region sequence.

91. The polypeptide chain of claim 76, wherein one or both of the dimerization domains comprise an engineered amino acid sequence that promotes interaction between the dimerization domains.

92. The polypeptide chain of claim 91, wherein the engineered amino acid sequence comprises a region selected from the group consisting of: a complementary hydrophobic region, a complementary hydrophilic region, and a compatible protein-protein interaction domain.

93. A first polypeptide chain comprising an antibody heavy chain variable region sequence specific for a first target C terminally conjugated to a first polypeptide sequence comprising a first VpreB sequence, wherein the N-terminus of the antibody heavy chain variable region sequence specific for a first target is conjugated to a dimerization domain.

94. The polypeptide chain of claim 93 associated with a second polypeptide chain comprising a first polypeptide sequence that comprises a second VpreB sequence, wherein the C-terminus of the first polypeptide sequence comprising the second VpreB sequence is conjugated to an antibody heavy chain variable region sequence specific for a second target and the N-terminus of the first polypeptide sequence comprising the second VpreB sequence is conjugated to a dimerization domain.

95-97. (canceled)

98. A heteromeric bispecific binding protein comprising the first and second polypeptide chains of claim 94, associated with each other.

99-108. (canceled)

109. A heteromeric trispecific binding protein comprising a first polypeptide chain comprising an antibody heavy chain variable region sequence specific for a first target, C-terminally conjugated to a polypeptide sequence comprising a first VpreB sequence, wherein the first polypeptide chain is associated with
a) a second polypeptide chain comprising a polypeptide sequence that comprises a second VpreB sequence conjugated to the N-terminus of an antibody heavy chain comprising a variable region sequence specific for a second target; and
b) a third polypeptide chain comprising a polypeptide sequence that comprises a third VpreB sequence conjugated to the N-terminus of an antibody heavy chain comprising a variable region sequence specific for a third target.

110-127. (canceled)


SEQUENCE LISTING (250 sequences)

SEQ ID NO: 1 - protein, 145 aa, Homo sapiens
MSWAPVLLMLFVYCTGCGPQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSEKEEREREWEEEMEPTAARTRVP

SEQ ID NO: 2 - protein, 142 aa, Mus sp.
MAWTSVLLMLLAHLTGCGPQPMVHQPPSASSSLGATIRLSCTLSNDHNIGIYSIYWYQQRPGHPPRFLLRYFSHSDKHQGPDIPPRFSGSKDTARNLGYLSISELQPEDEAVYYCAVGLRSHEKKRMEREWEGEKSYTDLGS

SEQ ID NO: 3 - protein, 171 aa, Mus sp.
MAWTSVLLMLLAHLTGKGTLGVQGFLAPPVALLCPSDGHASIFSGCGPQPMVHQPPSASSSLGATIRLSCTLSNDHNIGIYSIYWYQQRPGHPPRFLLRYFSHSDKHQGPDIPPRFSGSKDTARNLGYLSISELQPEDEAVYYCAVGLRSHEKKRMEREWEGEKSYTDLGS

SEQ ID NO: 4 - protein, 123 aa, Homo sapiens
MACRCLSFLLMGTFLSVSQTVLAQLDALLVFPGQVAQLSCTLSPQHVTIRDYGVSWYQQRAGSAPRYLLYYRSEEDHHRPADIPDRFSAAKDEAHNACVLTISPVQPEDDADYYCSVGYGFSP

SEQ ID NO: 5 - protein, 119 aa, Artificial Sequence
  source: /note=“Description of Artificial Sequence: Synthetic polypeptide”
MSWAPVLLMLFVYCTGCGPQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGA

SEQ ID NO: 6 - protein, 146 aa, Artificial Sequence
  source: /note=“Description of Artificial Sequence: Synthetic polypeptide”
METDTLLLWVLLLWVPGSTGQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSEKEEREREWEEEMEPTAARTRVP

SEQ ID NO: 7 - protein, 209 aa, Mus sp.
MKLRVGQTLGTIPRQCEVLLLLLLLGLVDGVHHILSPSSAERSRAVGPGASVGSNRPSLWALPGRLLFQIIPRGAGPRCSPHRLPSKPQFWYVFGGGTQLTILGQPKSDPLVTLFLPSLKNLQPTRPHVVCLVSEFYPGTLVVDWKVDGVPVTQGVETTQPSKQTNNKYMVSSYLTLISDQWMPHSRYSCRVTHEGNTVEKSVSPAECS

SEQ ID NO: 8 - protein, 213 aa, Homo sapiens
MRPGTGQGGLEAPGEPGPNLRQRWPLLLLGLAVVTHGLLRPTAASQSRALGPGAPGGSSRSSLRSRWGRFLLQRGSWTGPRCWPRGFQSKHNSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 9 - protein, 158 aa, Artificial Sequence
  source: /note=“Description of Artificial Sequence: Synthetic polypeptide”
MRPGTGQGGLEAPGEPGPNLRQRWPLLLLGLAVVTHGSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 10 - protein, 141 aa, Artificial Sequence
  source: /note=“Description of Artificial Sequence: Synthetic polypeptide”
METDTLLLWVLLLWVPGSTGSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 11 - DNA, 711 bp, Homo sapiens
  CDS (8)..(547)
cagcaagatggtgttgcagacccaggtcttcatttctctgttgctctggatctctggtgcctacggggacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctcccacagtgcttcagcctcgaacacaaacctcctccccatacgctgggccagtaggtctttgctgcagcagctgcttcctctgcacacagcccccaacatgcatgcttcctctgtgtgttggggaggtcactctcttgatttattcgttggagggtttgcagggcccaggattaaattaagagacttgacttttgctggatctctttttgtagaagattattaaagcaaaatgttgtaaagatcccttagagacattgtcaggagtttttgtgttacaggaacctgcatgtttcacatggacacatcacatgaccgagccaaatagatttatctttactct

SEQ ID NO: 12 - protein, 180 aa, Homo sapiens
MVLQTQVFISLLLWISGAYGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPPTVLQPRTQTSSPYAGPVGLCCSSCFLCTQPPTCMLPLCVGEVTLLIYSLEGLQGPGLN

SEQ ID NO: 13 - protein, 103 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFPPTVLPTRT

SEQ ID NO: 14 - protein, 103 aa, Homo sapiens
DIQMTQSPSSLSASVGDRVTITCRASQGISNYLAWFQQKPGKAPKSLIYAASSLQSGVPSRFSGSGSGTDFTLTISSLQPEDFATYYCQQYNSYPPTVLHTQT

SEQ ID NO: 15 - protein, 110 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPPTVLHTRTTPREADV

SEQ ID NO: 16 - protein, 98 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLPPTV

SEQ ID NO: 17 - protein, 101 aa, Homo sapiens
DIQMIQSPSFLSASVGDRVSIICWASEGISSNLAWYLQKPGKSPKLFLYDAKDLHPGVSSRFSGRGSGTDFTLTIISLKPEDFAAYYCKQDFSYPPTGLQA

SEQ ID NO: 18 - protein, 135 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPPTVVQPLTETSSWGCPVAHMCCLSGEQLSRVSESA

SEQ ID NO: 19 - protein, 106 aa, Homo sapiens
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPPTVVQP

SEQ ID NO: 20 - protein, 132 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTVIPHETKTPTRPSVFTRLLYQLLPLQTASGVATQC

SEQ ID NO: 21 - protein, 118 aa, Homo sapiens
EIVLTQSPATLSLSPGERATLSCRASQGVSSYLAWYQQKPGQAPRLLIYDASNRATGIPARFSGSGPGTDFTLTISSLEPEDFAVYYCQQRSNWHPTVIPHETKTPTRPSVFTRLLYQ

SEQ ID NO: 22 - protein, 115 aa, Homo sapiens
ETTLTQSPAFMSATPGDKVNISCKASQDIDDDMNWYQQKPGEAAIFIIQEATTLVPGIPPRFSGSGYGTDFTLTINNIESEDAAYYFCLQHDNFPLTVIHPVQKPPSSLSGIASA

SEQ ID NO: 23 - protein, 158 aa, Homo sapiens
DVVMTQSPAFLSVTPGEKVTITCQASEGIGNYLYWYQQKPDQAPKLLIKYASQSISGVPSRFSGSGSGTDFTFTISSLEAEDAATYYCQQGNKHPHTVLQPKTKISSAWRNRETEQYPVFMILAGAVGEIIYQIPSHMAHSAELTPKSQCLTLSSLPT

SEQ ID NO: 24 - protein, 103 aa, Homo sapiens
DIVLTQSPASLAVSPGQRATITCRASESVSFLGINLIHWYQQKPGQPPKLLIYQASNKDTGVPARFSGSGSGTDFTLTINPVEANDTANYYCLQSKNFPPTVL

SEQ ID NO: 25 - DNA, 408 bp, Homo sapiens
  CDS (1)..(405)
gtgagaagggtttttgttcagcaagacaatggagagctcacactgtggtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgttag

SEQ ID NO: 26 - protein, 135 aa, Homo sapiens
VRRVFVQQDNGELTLWWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 27 - protein, 119 aa, Artificial Sequence
  source: /note=“Description of Artificial Sequence: Synthetic polypeptide”
WTFGQGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 28 - protein, 119 aa, Artificial Sequence
  source: /note=“Description of Artificial Sequence: Synthetic polypeptide”
YTFGQGTKLEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 29 - protein, 119 aa, Artificial Sequence
  source: /note=“Description of Artificial Sequence: Synthetic polypeptide”
FTFGPGTKVDIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 30 - protein, 119 aa, Artificial Sequence
  source: /note=“Description of Artificial Sequence: Synthetic polypeptide”
LTFGGGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 31 - protein, 119 aa, Artificial Sequence
  source: /note=“Description of Artificial Sequence: Synthetic polypeptide”
ITFGQGTRLEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 32 - protein, 154 aa, Artificial Sequence [sequence omitted]

SEQ ID NO: 33 - protein, 138 aa, Artificial Sequence
  source: /note=“Description of Artificial Sequence: Synthetic polypeptide”
METDTLLLWVLLLWVPGSTGWTFGQGTKVEIKRTVAAPSVFIFPPSDEQLKSGTASVVCLLNNFYPREAKVQWKVDNALQGNSQESVTEQDSKDSTYSLSSTLTLSKADYEKHKLYACEVTHQGLSSPVTKSFNRGEC

SEQ ID NO: 34 - protein, 132 aa, Artificial Sequence
  source: /note=“Description of Artificial Sequence: Synthetic polypeptide”
METDTLLLWVLLLWVPGSTGTKVEIKRTVAAPSVFIFPPSDEQLKSGTASVVCLLNNFYPREAKVQWKVDNALQGNSQESVTEQDSKDSTYSLSSTLTLSKADYEKHKLYACEVTHQGLSSPVTKSFNRGEC

SEQ ID NO: 35 - protein, 243 aa, Artificial Sequence
  source: /note=“Description of Artificial Sequence: Synthetic polypeptide”
METDTLLLWVLLLWVPGSTGQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 36 - protein, 149 aa, Artificial Sequence
  source: /note=“Description of Artificial Sequence: Synthetic polypeptide”
METDTLLLWVLLLWVPGSTGQPVLHQPPAMSSALGTTIRLTCTLRNDHDIGVYSVYWYQQRPGHPPRFLLRYFSQSDKSQGPQVPPRFSGSKDVARNRGYLSISVTHVFGSGTQLTVLSQQVQLVQSVTHVFGSGTQLTVLSQQVQLVQ

SEQ ID NO: 37 - protein, 247 aa, Artificial Sequence
  source: /note=“Description of Artificial Sequence: Synthetic polypeptide”
GQGTLVTVSSASQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSRCWPRGFQSKHNSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 38 - protein, 248 aa, Artificial Sequence
  source: /note=“Description of Artificial Sequence: Synthetic polypeptide”
GQGTLVTVSSASTQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSRCWPRGFQSKHNSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 39 - protein, 249 aa, Artificial Sequence
  source: /note=“Description of Artificial Sequence: Synthetic polypeptide”
GQGTLVTVSSASTKQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSRCWPRGFQSKHNSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 40 - protein, 249 aa, Artificial Sequence; identical to SEQ ID NO: 39
  source: /note=“Description of Artificial Sequence: Synthetic polypeptide”

SEQ ID NO: 41 - protein, 250 aa, Artificial Sequence
  source: /note=“Description of Artificial Sequence: Synthetic polypeptide”
GQGTLVTVSSASTKGQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSRCWPRGFQSKHNSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 42 - protein, 251 aa, Artificial Sequence
  source: /note=“Description of Artificial Sequence: Synthetic polypeptide”
GQGTLVTVSSASTKGPQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSRCWPRGFQSKHNSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 43 - protein, 252 aa, Artificial Sequence
  source: /note=“Description of Artificial Sequence: Synthetic polypeptide”
GQGTLVTVSSASTKGPSQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSRCWPRGFQSKHNSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 44 - protein, 253 aa, Artificial Sequence
  source: /note=“Description of Artificial Sequence: Synthetic polypeptide”
GQGTLVTVSSASTKGPSVQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSRCWPRGFQSKHNSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 45 - protein, 254 aa, Artificial Sequence
  source: /note=“Description of Artificial Sequence: Synthetic polypeptide”
GQGTLVTVSSASTKGPSVFQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSRCWPRGFQSKHNSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 46 - protein, 255 aa, Artificial Sequence
  source: /note=“Description of Artificial Sequence: Synthetic polypeptide”
GQGTLVTVSSASTKGPSVFPQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSRCWPRGFQSKHNSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 47 - protein, 125 aa, Artificial Sequence
  source: /note=“Description of Artificial Sequence: Synthetic polypeptide”
MSWAPVLLMLFVYCTGCGPQPVLHQPPAMSSALGTTIRLTCTLRNDHDIGVYSVYWYQQRPGHPPRFLLRYFSQSDKSQGPQVPPRFSGSKDVARNRGYLSISVTHVFGSGTQLTVLSQQVQLVQ

SEQ ID NO: 48 - protein, 126 aa, Artificial Sequence
  source: /note=“Description of Artificial Sequence: Synthetic polypeptide”
MSWAPVLLMLFVYCTGCGPQPVLHQPPAMSSALGTTIRLTCTLRNDHDIGVYSVYWYQQRPGHPPRFLLRYFSQSDKSQGPQVPPRFSGSKDVARNRGYLSISVTHVFGSGTQLTVLSQPQVQLVQ

SEQ ID NO: 49 - protein, 127 aa, Artificial Sequence
  source: /note=“Description of Artificial Sequence: Synthetic polypeptide”
MSWAPVLLMLFVYCTGCGPQPVLHQPPAMSSALGTTIRLTCTLRNDHDIGVYSVYWYQQRPGHPPRFLLRYFSQSDKSQGPQVPPRFSGSKDVARNRGYLSISVTHVFGSGTQLTVLSQPKQVQLVQ

SEQ ID NO: 50 - protein, 128 aa, Artificial Sequence
  source: /note=“Description of Artificial Sequence: Synthetic polypeptide”
MSWAPVLLMLFVYCTGCGPQPVLHQPPAMSSALGTTIRLTCTLRNDHDIGVYSVYWYQQRPGHPPRFLLRYFSQSDKSQGPQVPPRFSGSKDVARNRGYLSISVTHVFGSGTQLTVLSQPKAQVQLVQ

SEQ ID NO: 51 - protein, 129 aa, Artificial Sequence
  source: /note=“Description of Artificial Sequence: Synthetic polypeptide”
MSWAPVLLMLFVYCTGCGPQPVLHQPPAMSSALGTTIRLTCTLRNDHDIGVYSVYWYQQRPGHPPRFLLRYFSQSDKSQGPQVPPRFSGSKDVARNRGYLSISVTHVFGSGTQLTVLSQPKATQVQLVQ

SEQ ID NO: 52 - protein, 130 aa, Artificial Sequence
  source: /note=“Description of Artificial Sequence: Synthetic polypeptide”
MSWAPVLLMLFVYCTGCGPQPVLHQPPAMSSALGTTIRLTCTLRNDHDIGVYSVYWYQQRPGHPPRFLLRYFSQSDKSQGPQVPPRFSGSKDVARNRGYLSISVTHVFGSGTQLTVLSQPKATPQVQLVQ

SEQ ID NO: 53 - protein, 132 aa, Artificial Sequence
  source: /note=“Description of Artificial Sequence: Synthetic polypeptide”
MSWAPVLLMLFVYCTGCGPQPVLHQPPAMSSALGTTIRLTCTLRNDHDIGVYSVYWYQQRPGHPPRFLLRYFSQSDKSQGPQVPPRFSGSKDVARNRGYLSISVTHVFGSGTQLTVLSQPKATPSVQVQLVQ

SEQ ID NO: 54 - protein, 133 aa, Artificial Sequence
  source: /note=“Description of Artificial Sequence: Synthetic polypeptide”
MSWAPVLLMLFVYCTGCGPQPVLHQPPAMSSALGTTIRLTCTLRNDHDIGVYSVYWYQQRPGHPPRFLLRYFSQSDKSQGPQVPPRFSGSKDVARNRGYLSISVTHVFGSGTQLTVLSQPKATPSVTQVQLVQ

SEQ ID NO: 55 - protein, 138 aa, Artificial Sequence
  source: /note=“Description of Artificial Sequence: Synthetic polypeptide”
MSWAPVLLMLFVYCTGCGPQPVLHQPPAMSSALGTTIRLTCTLRNDHDIGVYSVYWYQQRPGHPPRFLLRYFSQSDKSQGPQVPPRFSGSKDVARNRGYLSISVTHVFGSGTQLTVLSQPKATPSVTGGGGSQVQLVQ

SEQ ID NO: 56 - protein, 380 aa, Artificial Sequence
  source: /note=“Description of Artificial Sequence: Synthetic polypeptide”
GQGTLVTVSSGGGGSGGGGSGGGGSQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSGAQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSRCWPRGFQSKHNSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 57 - protein, 467 aa, Artificial Sequence
  source: /note=“Description of Artificial Sequence: Synthetic polypeptide”
GQGTLVTVSSASTKGPSVFPQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 466 aa, Artificial Sequence
  source: /note=“Description of Artificial Sequence: Synthetic polypeptide”
GQGTLVTVSSASTKGPSVFQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 465 aa, Artificial Sequence
  source: /note=“Description of Artificial Sequence: Synthetic polypeptide”
GQGTLVTVSSASTKGPSVQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 464 aa, Artificial Sequence
  source: /note=“Description of Artificial Sequence: Synthetic polypeptide”
GQGTLVTVSSASTKGPSQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 463 aa, Artificial Sequence
  source: /note=“Description of Artificial Sequence: Synthetic polypeptide”
GQGTLVTVSSASTKGPQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 462 aa, Artificial Sequence
  source: /note=“Description of Artificial Sequence: Synthetic polypeptide”
GQGTLVTVSSASTKGQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 461 aa, Artificial Sequence
  source: /note=“Description of Artificial Sequence: Synthetic polypeptide”
GQGTLVTVSSASTKQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 460 aa, Artificial Sequence
  source: /note=“Description of Artificial Sequence: Synthetic polypeptide”
GQGTLVTVSSASTQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 459 aa, Artificial Sequence
  source: /note=“Description of Artificial Sequence: Synthetic polypeptide”
GQGTLVTVSSASQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - skipped

SEQ ID NO: 67 - protein, 22 aa, Artificial Sequence
  source: /note=“Description of Artificial Sequence: Synthetic peptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (13)..(22): Any amino acid
  VARIANT (13)..(22): /replace=“ ”
  misc_feature (13)..(22): /note=“Residues given in the sequence have no preference with respect to those in the annotations for said positions”
  misc_feature (13)..(22): /note=“This region may encompass 0 to 10 residues”
  source: /note=“see specification as filed for detailed description of substitutions and preferred embodiments”
XXXXXXXXXXASXXXXXXXXXX

SEQ ID NO: 68 - protein, 23 aa, Artificial Sequence
  source: /note=“Description of Artificial Sequence: Synthetic peptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (14)..(23): Any amino acid
  VARIANT (14)..(23): /replace=“ ”
  misc_feature (14)..(23): /note=“Residues given in the sequence have no preference with respect to those in the annotations for said positions”
  misc_feature (14)..(23): /note=“This region may encompass 0 to 10 residues”
  source: /note=“see specification as filed for detailed description of substitutions and preferred embodiments”
XXXXXXXXXXASTXXXXXXXXXX

SEQ ID NO: 69 - protein, 24 aa, Artificial Sequence
  source: /note=“Description of Artificial Sequence: Synthetic peptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (15)..(24): Any amino acid
  VARIANT (15)..(24): /replace=“ ”
  misc_feature (15)..(24): /note=“Residues given in the sequence have no preference with respect to those in the annotations for said positions”
  misc_feature (15)..(24): /note=“This region may encompass 0 to 10 residues”
  source: /note=“see specification as filed for detailed description of substitutions and preferred embodiments”
XXXXXXXXXXASTKXXXXXXXXXX

SEQ ID NO: 70 - protein, 25 aa, Artificial Sequence
  source: /note=“Description of Artificial Sequence: Synthetic peptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (16)..(25): Any amino acid
  VARIANT (16)..(25): /replace=“ ”
  misc_feature (16)..(25): /note=“Residues given in the sequence have no preference with respect to those in the annotations for said positions”
  misc_feature (16)..(25): /note=“This region may encompass 0 to 10 residues”
  source: /note=“see specification as filed for detailed description of substitutions and preferred embodiments”
XXXXXXXXXXASTKGXXXXXXXXXX

SEQ ID NO: 71 - protein, 26 aa, Artificial Sequence
  source: /note=“Description of Artificial Sequence: Synthetic peptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (17)..(26): Any amino acid
  VARIANT (17)..(26): /replace=“ ”
  misc_feature (17)..(26): /note=“Residues given in the sequence have no preference with respect to those in the annotations for said positions”
  misc_feature (17)..(26): /note=“This region may encompass 0 to 10 residues”
  source: /note=“see specification as filed for detailed description of substitutions and preferred embodiments”
XXXXXXXXXXASTKGPXXXXXXXXXX

SEQ ID NO: 72 - protein, 27 aa, Artificial Sequence
  source: /note=“Description of Artificial Sequence: Synthetic peptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (18)..(27): Any amino acid
  VARIANT (18)..(27): /replace=“ ”
  misc_feature (18)..(27): /note=“Residues given in the sequence have no preference with respect to those in the annotations for said positions”
  misc_feature (18)..(27): /note=“This region may encompass 0 to 10 residues”
  source: /note=“see specification as filed for detailed description of substitutions and preferred embodiments”
XXXXXXXXXXASTKGPSXXXXXXXXXX

SEQ ID NO: 73 - protein, 28 aa, Artificial Sequence
  source: /note=“Description of Artificial Sequence: Synthetic peptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (19)..(28): Any amino acid
  VARIANT (19)..(28): /replace=“ ”
  misc_feature (19)..(28): /note=“Residues given in the sequence have no preference with respect to those in the annotations for said positions”
  misc_feature (19)..(28): /note=“This region may encompass 0 to 10 residues”
  source: /note=“see specification as filed for detailed description of substitutions and preferred embodiments”
XXXXXXXXXXASTKGPSVXXXXXXXXXX

SEQ ID NO: 74 - protein, 29 aa, Artificial Sequence
  source: /note=“Description of Artificial Sequence: Synthetic peptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (20)..(29): Any amino acid
  VARIANT (20)..(29): /replace=“ ”
  misc_feature (20)..(29): /note=“Residues given in the sequence have no preference with respect to those in the annotations for said positions”
  misc_feature (20)..(29): /note=“This region may encompass 0 to 10 residues”
  source: /note=“see specification as filed for detailed description of substitutions and preferred embodiments”
XXXXXXXXXXASTKGPSVFXXXXXXXXXX

SEQ ID NO: 75 - protein, 30 aa, Artificial Sequence
  source: /note=“Description of Artificial Sequence: Synthetic polypeptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (21)..(30): Any amino acid
  VARIANT (21)..(30): /replace=“ ”
  misc_feature (21)..(30): /note=“Residues given in the sequence have no preference with respect to those in the annotations for said positions”
  misc_feature (21)..(30): /note=“This region may encompass 0 to 10 residues”
  source: /note=“see specification as filed for detailed description of substitutions and preferred embodiments”
XXXXXXXXXXASTKGPSVFPXXXXXXXXXX

SEQ ID NO: 76 - protein, 4 aa, Artificial Sequence
  source: /note=“Description of Artificial Sequence: Synthetic peptide”
TVSS

SEQ ID NO: 77 - protein, 5 aa, Artificial Sequence
  source: /note=“Description of Artificial Sequence: Synthetic peptide”
VTVSS

SEQ ID NO: 78 - protein, 6 aa, Artificial Sequence
  source: /note=“Description of Artificial Sequence: Synthetic peptide”
LVTVSS

SEQ ID NO: 79 - protein, 7 aa, Artificial Sequence
  source: /note=“Description of Artificial Sequence: Synthetic peptide”
TLVTVSS

SEQ ID NO: 80 - protein, 8 aa, Artificial Sequence
  source: /note=“Description of Artificial Sequence: Synthetic peptide”
GTLVTVSS

SEQ ID NO: 81 - protein, 9 aa, Artificial Sequence
  source: /note=“Description of Artificial Sequence: Synthetic peptide”
QGTLVTVSS

SEQ ID NO: 82 - protein, 10 aa, Artificial Sequence
  source: /note=“Description of Artificial Sequence: Synthetic peptide”
GQGTLVTVSS

SEQ ID NO: 83 - protein, 5 aa, Artificial Sequence
  source: /note=“Description of Artificial Sequence: Synthetic peptide”
QPVLH

SEQ ID NO: 84 - protein, 6 aa, Artificial Sequence
  source: /note=“Description of Artificial Sequence: Synthetic peptide”
QPVLHQ

SEQ ID NO: 85 - protein, 7 aa, Artificial Sequence
  source: /note=“Description of Artificial Sequence: Synthetic peptide”
QPVLHQP

SEQ ID NO: 86 - protein, 8 aa, Artificial Sequence
  source: /note=“Description of Artificial Sequence: Synthetic peptide”
QPVLHQPP

SEQ ID NO: 87 - protein, 9 aa, Artificial Sequence
  source: /note=“Description of Artificial Sequence: Synthetic peptide”
QPVLHQPPA

SEQ ID NO: 88 - protein, 10 aa, Artificial Sequence
  source: /note=“Description of Artificial Sequence: Synthetic peptide”
QPVLHQPPAM

SEQ ID NO: 89 - protein, 18 aa, Artificial Sequence
  source: /note=“Description of Artificial Sequence: Synthetic peptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (13)..(18): Any amino acid
  VARIANT (13)..(18): /replace=“ ”
  misc_feature (13)..(18): /note=“Residues given in the sequence have no preference with respect to those in the annotations for said positions”
  misc_feature (13)..(18): /note=“This region may encompass 0 to 6 residues”
  source: /note=“see specification as filed for detailed description of substitutions and preferred embodiments”
XXXXXXXXXXSQXXXXXX

SEQ ID NO: 90 - protein, 19 aa, Artificial Sequence
  source: /note=“Description of Artificial Sequence: Synthetic peptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (14)..(19): Any amino acid
  VARIANT (14)..(19): /replace=“ ”
  misc_feature (14)..(19): /note=“Residues given in the sequence have no preference with respect to those in the annotations for said positions”
  misc_feature (14)..(19): /note=“This region may encompass 0 to 6 residues”
  source: /note=“see specification as filed for detailed description of substitutions and preferred embodiments”
XXXXXXXXXXSQPXXXXXX

SEQ ID NO: 91 - protein, 20 aa, Artificial Sequence
  source: /note=“Description of Artificial Sequence: Synthetic peptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (15)..(20): Any amino acid
  VARIANT (15)..(20): /replace=“ ”
  misc_feature (15)..(20): /note=“Residues given in the sequence have no preference with respect to those in the annotations for said positions”
  misc_feature (15)..(20): /note=“This region may encompass 0 to 6 residues”
  source: /note=“see specification as filed for detailed description of substitutions and preferred embodiments”
XXXXXXXXXXSQPKXXXXXX

SEQ ID NO: 92 - protein, 21 aa, Artificial Sequence
  source: /note=“Description of Artificial Sequence: Synthetic peptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (16)..(21): Any amino acid
  VARIANT (16)..(21): /replace=“ ”
  misc_feature (16)..(21): /note=“Residues given in the sequence have no preference with respect to those in the annotations for said positions”
  misc_feature (16)..(21): /note=“This region may encompass 0 to 6 residues”
  source: /note=“see specification as filed for detailed description of substitutions and preferred embodiments”
XXXXXXXXXXSQPKAXXXXXX

SEQ ID NO: 93 - protein, 22 aa, Artificial Sequence
  source: /note=“Description of Artificial Sequence: Synthetic peptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (17)..(22): Any amino acid
  VARIANT (17)..(22): /replace=“ ”
  misc_feature (17)..(22): /note=“Residues given in the sequence have no preference with respect to those in the annotations for said positions”
  misc_feature (17)..(22): /note=“This region may encompass 0 to 6 residues”
  source: /note=“see specification as filed for detailed description of substitutions and preferred embodiments”
XXXXXXXXXXSQPKATXXXXXX

SEQ ID NO: 94 - protein, 23 aa, Artificial Sequence
  source: /note=“Description of Artificial Sequence: Synthetic peptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (18)..(23): Any amino acid
  VARIANT (18)..(23): /replace=“ ”
  misc_feature (18)..(23): /note=“Residues given in the sequence have no preference with respect to those in the annotations for said positions”
  misc_feature (18)..(23): /note=“This region may encompass 0 to 6 residues”
  source: /note=“see specification as filed for detailed description of substitutions and preferred embodiments”
XXXXXXXXXXSQPKATPXXXXXX

SEQ ID NO: 95 - protein, 24 aa, Artificial Sequence
  source: /note=“Description of Artificial Sequence: Synthetic peptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (19)..(24): Any amino acid
  VARIANT (19)..(24): /replace=“ ”
  misc_feature (19)..(24): /note=“Residues given in the sequence have no preference with respect to those in the annotations for said positions”
  misc_feature (19)..(24): /note=“This region may encompass 0 to 6 residues”
  source: /note=“see specification as filed for detailed description of substitutions and preferred embodiments”
XXXXXXXXXXSQPKATPSXXXXXX

SEQ ID NO: 96 - protein, 25 aa, Artificial Sequence
  source: /note=“Description of Artificial Sequence: Synthetic peptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (20)..(25): Any amino acid
  VARIANT (20)..(25): /replace=“ ”
  misc_feature (20)..(25): /note=“Residues given in the sequence have no preference with respect to those in the annotations for said positions”
  misc_feature (20)..(25): /note=“This region may encompass 0 to 6 residues”
  source: /note=“see specification as filed for detailed description of substitutions and preferred embodiments”
XXXXXXXXXXSQPKATPSVXXXXXX

SEQ ID NO: 97 - protein, 26 aa, Artificial Sequence
  source: /note=“Description of Artificial Sequence: Synthetic peptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (21)..(26): Any amino acid
  VARIANT (21)..(26): /replace=“ ”
  misc_feature (21)..(26): /note=“Residues given in the sequence have no preference with respect to those in the annotations for said positions”
  misc_feature (21)..(26): /note=“This region may encompass 0 to 6 residues”
  source: /note=“see specification as filed for detailed description of substitutions and preferred embodiments”
XXXXXXXXXXSQPKATPSVTXXXXXX

SEQ ID NO: 98 - protein, 31 aa, Artificial Sequence
  source: /note=“Description of Artificial Sequence: Synthetic polypeptide”
  MOD_RES (1)..(10): Any amino acid
  VARIANT (1)..(10): /replace=“ ”
  misc_feature (1)..(10): /note=“Residues given in the sequence have no preference with respect to those in the annotations for said positions”
  misc_feature (1)..(10): /note=“This region may encompass 0 to 10 residues”
  MOD_RES (26)..(31): Any amino acid
  VARIANT (26)..(31): /replace=“ ”
  misc_feature (26)..(31): /note=“Residues given in the sequence have no preference with respect to those in the annotations for said positions”
  misc_feature (26)..(31): /note=“This region may encompass 0 to 6 residues”
  source: /note=“see specification as filed for detailed description of substitutions and preferred embodiments”
XXXXXXXXXXSQPKATPSVTGGGGSXXXXXX

SEQ ID NO: 99 - protein, 4 aa, Artificial Sequence
  source: /note=“Description of Artificial Sequence: Synthetic peptide”
LTVL

SEQ ID NO: 100 - protein, 5 aa, Artificial Sequence
  source: /note=“Description of Artificial Sequence: Synthetic peptide”
QLTVL

SEQ ID NO: 101 - protein, 6 aa, Artificial Sequence
  source: /note=“Description of Artificial Sequence: Synthetic peptide”
TQLTVL

SEQ ID NO: 102 - protein, 7 aa, Artificial Sequence
  source: /note=“Description of Artificial Sequence: Synthetic peptide”
GTQLTVL

SEQ ID NO: 103 - protein, 8 aa, Artificial Sequence
  source: /note=“Description of Artificial Sequence: Synthetic peptide”
SGTQLTVL

SEQ ID NO: 104 - protein, 9 aa, Artificial Sequence
  source: /note=“Description of Artificial Sequence: Synthetic peptide”
GSGTQLTVL

SEQ ID NO: 105 - protein, 4 aa, Artificial Sequence
  source: /note=“Description of Artificial Sequence: Synthetic peptide”
QVQL

SEQ ID NO: 106 - protein, 5 aa, Artificial Sequence
  source: /note=“Description of Artificial Sequence: Synthetic peptide”
QVQLV

SEQ ID NO: 107 - protein, 6 aa, Artificial Sequence
  source: /note=“Description of Artificial Sequence: Synthetic peptide”
QVQLVQ

SEQ ID NO: 108 - protein, 5 aa, Artificial Sequence
  source: /note=“Description of Artificial Sequence: Synthetic peptide”
GGGGS

SEQ ID NO: 109 - protein, 4 aa, Artificial Sequence
  source: /note=“Description of Artificial Sequence: Synthetic peptide”
  source: /note=“see specification as filed for detailed description of substitutions and preferred embodiments”
GGGS

SEQ ID NO: 110 - protein, 5 aa, Artificial Sequence
  source: /note=“Description of Artificial Sequence: Synthetic peptide”
  source: /note=“see specification as filed for detailed description of substitutions and preferred embodiments”
GGGGS

SEQ ID NO: 111 - protein, 8 aa, Artificial Sequence
  source: /note=“Description of Artificial Sequence: Synthetic peptide”
GGGSGGGS

SEQ ID NO: 112 - protein, 4 aa, Artificial Sequence
  source: /note=“Description of Artificial Sequence: Synthetic peptide”
ASTK

SEQ ID NO: 113 - protein, 5 aa, Artificial Sequence
  source: /note=“Description of Artificial Sequence: Synthetic peptide”
ASTKG

SEQ ID NO: 114 - protein, 6 aa, Artificial Sequence
  source: /note=“Description of Artificial Sequence: Synthetic peptide”
ASTKGP

SEQ ID NO: 115 - protein, 7 aa, Artificial Sequence
  source: /note=“Description of Artificial Sequence: Synthetic peptide”
ASTKGPS

SEQ ID NO: 116 - protein, 8 aa, Artificial Sequence
  source: /note=“Description of Artificial Sequence: Synthetic peptide”
ASTKGPSV

SEQ ID NO: 117 - protein, 9 aa, Artificial Sequence
  source: /note=“Description of Artificial Sequence: Synthetic peptide”
ASTKGPSVF

SEQ ID NO: 118 - protein, 10 aa, Artificial Sequence
  source: /note=“Description of Artificial Sequence: Synthetic peptide”
ASTKGPSVFP

SEQ ID NO: 119 - protein, 15 aa, Artificial Sequence
  source: /note=“Description of Artificial Sequence: Synthetic peptide”
GGGGSGGGGSGGGGS

SEQ ID NO: 120 - protein, 4 aa, Artificial Sequence
  source: /note=“Description of Artificial Sequence: Synthetic peptide”
SQPK

SEQ ID NO: 121 - protein, 5 aa, Artificial Sequence
  source: /note=“Description of Artificial Sequence: Synthetic peptide”
SQPKA

SEQ ID NO: 122 - protein, 6 aa, Artificial Sequence
  source: /note=“Description of Artificial Sequence: Synthetic peptide”
SQPKAT

SEQ ID NO: 123 - protein, 7 aa, Artificial Sequence
  source: /note=“Description of Artificial Sequence: Synthetic peptide”
SQPKATP

SEQ ID NO: 124 - protein, 8 aa, Artificial Sequence
  source: /note=“Description of Artificial Sequence: Synthetic peptide”
SQPKATPS

SEQ ID NO: 125 - protein, 9 aa, Artificial Sequence
  source: /note=“Description of Artificial Sequence: Synthetic peptide”
SQPKATPSV

SEQ ID NO: 126 - protein, 10 aa, Artificial Sequence
  source: /note=“Description of Artificial Sequence: Synthetic peptide”
SQPKATPSVT

SEQ ID NO: 127 - protein, 15 aa, Artificial Sequence
  source: /note=“Description of Artificial Sequence: Synthetic peptide”
SQPKATPSVTGGGGS

SEQ ID NO: 128 - protein, 4 aa, Artificial Sequence
  source: /note=“Description of Artificial Sequence: Synthetic peptide”
QPVL

SEQ ID NO: 129 - protein, 22 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (13)..(22): Any amino acid
  VARIANT (14)..(22): /replace=“ ”
  misc_feature (14)..(22): /note=“Residues given in the sequence have no preference with respect to those in the annotations for said positions”
  misc_feature (13)..(22): /note=“This region may encompass 1 to 10 residues”
  source: /note=“see specification as filed for detailed description of substitutions and preferred embodiments”
XXXXXXXXXXGAXXXXXXXXXX

SEQ ID NO: 130 - protein, 4 aa, Artificial Sequence
  source: /note=“Description of Artificial Sequence: Synthetic peptide”
TVLS

SEQ ID NO: 131 - protein, 5 aa, Artificial Sequence
  source: /note=“Description of Artificial Sequence: Synthetic peptide”
LTVLS

SEQ ID NO: 132 - protein, 6 aa, Artificial Sequence
  source: /note=“Description of Artificial Sequence: Synthetic peptide”
QLTVLS

SEQ ID NO: 133 - protein, 7 aa, Artificial Sequence
  source: /note=“Description of Artificial Sequence: Synthetic peptide”
TQLTVLS

SEQ ID NO: 134 - protein, 8 aa, Artificial Sequence
  source: /note=“Description of Artificial Sequence: Synthetic peptide”
GTQLTVLS

SEQ ID NO: 135 - protein, 9 aa, Artificial Sequence
  source: /note=“Description of Artificial Sequence: Synthetic peptide”
SGTQLTVLS

SEQ ID NO: 136 - protein, 10 aa, Artificial Sequence
  source: /note=“Description of Artificial Sequence: Synthetic peptide”
GSGTQLTVLS

SEQ ID NO: 137 - protein, 4 aa, Artificial Sequence
  source: /note=“Description of Artificial Sequence: Synthetic peptide”
GSGT

SEQ ID NO: 138 - protein, 5 aa, Artificial Sequence
  source: /note=“Description of Artificial Sequence: Synthetic peptide”
GSGTQ

SEQ ID NO: 139 - protein, 6 aa, Artificial Sequence
  source: /note=“Description of Artificial Sequence: Synthetic peptide”
GSGTQL

SEQ ID NO: 140 - protein, 7 aa, Artificial Sequence
  source: /note=“Description of Artificial Sequence: Synthetic peptide”
GSGTQLT

SEQ ID NO: 141 - protein, 8 aa, Artificial Sequence
  source: /note=“Description of Artificial Sequence: Synthetic peptide”
GSGTQLTV

SEQ ID NO: 142 - protein, 9 aa, Artificial Sequence
  source: /note=“Description of Artificial Sequence: Synthetic peptide”
GSGTQLTVL

SEQ ID NO: 143 - protein, 10 aa, Artificial Sequence
  source: /note=“Description of Artificial Sequence: Synthetic peptide”
GSGTQLTVLS

SEQ ID NO: 144 - protein, 21 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (12)..(21): Any amino acid
  VARIANT (13)..(21): /replace=“ ”
  misc_feature (13)..(21): /note=“Residues given in the sequence have no preference with respect to those in the annotations for said positions”
  misc_feature (12)..(21): /note=“This region may encompass 1 to 10 residues”
  source: /note=“see specification as filed for detailed description of substitutions and preferred embodiments”
XXXXXXXXXXSXXXXXXXXXX

SEQ ID NO: 145 - protein, 4 aa, Artificial Sequence
  source: /note=“Description of Artificial Sequence: Synthetic peptide”
ASTK

SEQ ID NO: 146 - protein, 5 aa, Artificial Sequence
  source: /note=“Description of Artificial Sequence: Synthetic peptide”
ASTKG

SEQ ID NO: 147 - protein, 6 aa, Artificial Sequence
  source: /note=“Description of Artificial Sequence: Synthetic peptide”
ASTKGP

SEQ ID NO: 148 - protein, 7 aa, Artificial Sequence
  source: /note=“Description of Artificial Sequence: Synthetic peptide”
ASTKGPS

SEQ ID NO: 149 - protein, 8 aa, Artificial Sequence
  source: /note=“Description of Artificial Sequence: Synthetic peptide”
ASTKGPSV

SEQ ID NO: 150 - protein, 9 aa, Artificial Sequence
  source: /note=“Description of Artificial Sequence: Synthetic peptide”
ASTKGPSVF

SEQ ID NO: 151 - protein, 10 aa, Artificial Sequence
  source: /note=“Description of Artificial Sequence: Synthetic peptide”
ASTKGPSVFP

SEQ ID NO: 152 - protein, 243 aa, Artificial Sequence
  source: /note=“Description of Artificial Sequence: Synthetic polypeptide”
GQGTLVTVSSASTKGPSVFPQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 153 - protein, 242 aa, Artificial Sequence
  source: /note=“Description of Artificial Sequence: Synthetic polypeptide”
GQGTLVTVSSASTKGPSVFQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 154 - protein, 133 aa, Artificial Sequence
  Description of Artificial Sequence: Synthetic polypeptide
QPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSQPKATPSVTQVQLVQ

SEQ ID NO: 155 - protein, 138 aa, Artificial Sequence
  source: /note=“Description of Artificial Sequence: Synthetic polypeptide”
QPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSQPKATPSVTGGGGSQVQLVQ

SEQ ID NO: 156 - protein, 132 aa, Artificial Sequence
  source: /note=“Description of Artificial Sequence: Synthetic polypeptide”
QPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSQPKATPSVQVQLVQ

SEQ ID NO: 157 - protein, 131 aa, Artificial Sequence
  source: /note=“Description of Artificial Sequence: Synthetic polypeptide”
QPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSQPKATPSVTEVQL

SEQ ID NO: 158 - protein, 130 aa, Artificial Sequence
  source: /note=“Description of Artificial Sequence: Synthetic polypeptide”
QPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSQPKATPSVEVQL

SEQ ID NO: 159 - protein, 136 aa, Artificial Sequence
  source: /note=“Description of Artificial Sequence: Synthetic polypeptide”
QPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSQPKATPSVTGGGGSEVQL

SEQ ID NO: 160 - protein, 127 aa, Artificial Sequence
  source: /note=“Description of Artificial Sequence: Synthetic polypeptide”
QPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSQPKATPSVT

SEQ ID NO: 161 - protein, 131 aa, Artificial Sequence; identical to SEQ ID NO: 157
  source: /note=“Description of Artificial Sequence: Synthetic polypeptide”

SEQ ID NO: 162 - protein, 4 aa, Artificial Sequence
  source: /note=“Description of Artificial Sequence: Synthetic peptide”
MYYC

SEQ ID NO: 163 - protein, 5 aa, Artificial Sequence
  source: /note=“Description of Artificial Sequence: Synthetic peptide”
MYYCA

SEQ ID NO: 164 - protein, 6 aa, Artificial Sequence
  source: /note=“Description of Artificial Sequence: Synthetic peptide”
MYYCAM

SEQ ID NO: 165 - protein, 7 aa, Artificial Sequence
  source: /note=“Description of Artificial Sequence: Synthetic peptide”
MYYCAMG

SEQ ID NO: 166 - protein, 8 aa, Artificial Sequence
  source: /note=“Description of Artificial Sequence: Synthetic peptide”
MYYCAMGA

SEQ ID NO: 167 - protein, 9 aa, Artificial Sequence
  source: /note=“Description of Artificial Sequence: Synthetic peptide”
MYYCAMGAR

SEQ ID NO: 168 - protein, 10 aa, Artificial Sequence
  source: /note=“Description of Artificial Sequence: Synthetic peptide”
MYYCAMGARS

SEQ ID NO: 169-199 - skipped (31 entries)

SEQ ID NO: 200 - protein, 131 aa, Artificial Sequence
  source: /note=“Description of Artificial Sequence: Synthetic polypeptide”
MSWAPVLLMLFVYCTGCGPQPVLHQPPAMSSALGTTIRLTCTLRNDHDIGVYSVYWYQQRPGHPPRFLLRYFSQSDKSQGPQVPPRFSGSKDVARNRGYLSISVTHVFGSGTQLTVLSQPKATPSQVQLVQ

SEQ ID NO: 201 - protein, 130 aa, Artificial Sequence; identical to SEQ ID NO: 158
  source: /note=“Description of Artificial Sequence: Synthetic polypeptide”

SEQ ID NO: 202 - protein, 131 aa, Artificial Sequence; identical to SEQ ID NO: 157
  source: /note=“Description of Artificial Sequence: Synthetic polypeptide”

SEQ ID NO: 203-204 - skipped (2 entries)

SEQ ID NO: 205 - protein, 121 aa, Homo sapiens
QVQLVQSGAEVKKPGSSVKVSCKASGGTFSTYGISWVRQAPGQGLEWMGWITPITGHTTYAQKFQGRVTITADKSTSTAYMELSSLRSEDTAVYYCARDGATIWNAGLDYWGQGTLVTVSS

SEQ ID NO: 206 - protein, 124 aa, Homo sapiens
QVQLVQSGAEVKKPGASVKVSCKASGYTFSNYGMHWVRQAPGQGLEWMGGINVNSGGPNYAQKFQGRVTMTRDTSISTAYMELSRLRSDDTAVYYCARVGWSLDSSRFSGMDYWGQGTLVTVSS

SEQ ID NO: 207 - protein, 22 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (13)..(22): Any amino acid
  VARIANT (14)..(22): /replace=“ ”
  misc_feature (14)..(22): /note=“Residues given in the sequence have no preference with respect to those in the annotations for said positions”
  misc_feature (13)..(22): /note=“This region may encompass 1 to 10 residues”
  source: /note=“see specification as filed for detailed description of substitutions and preferred embodiments”
XXXXXXXXXXASXXXXXXXXXX

SEQ ID NO: 208 - protein, 23 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (14)..(23): Any amino acid
  VARIANT (15)..(23): /replace=“ ”
  misc_feature (15)..(23): /note=“Residues given in the sequence have no preference with respect to those in the annotations for said positions”
  misc_feature (14)..(23): /note=“This region may encompass 1 to 10 residues”
  source: /note=“see specification as filed for detailed description of substitutions and preferred embodiments”
XXXXXXXXXXASTXXXXXXXXXX

SEQ ID NO: 209 - protein, 24 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (15)..(24): Any amino acid
  VARIANT (16)..(24): /replace=“ ”
  misc_feature (16)..(24): /note=“Residues given in the sequence have no preference with respect to those in the annotations for said positions”
  misc_feature (15)..(24): /note=“This region may encompass 1 to 10 residues”
  source: /note=“see specification as filed for detailed description of substitutions and preferred embodiments”
XXXXXXXXXXASTKXXXXXXXXXX

SEQ ID NO: 210 - protein, 25 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (16)..(25): Any amino acid
  VARIANT (17)..(25): /replace=“ ”
  misc_feature (17)..(25): /note=“Residues given in the sequence have no preference with respect to those in the annotations for said positions”
  misc_feature (16)..(25): /note=“This region may encompass 1 to 10 residues”
  source: /note=“see specification as filed for detailed description of substitutions and preferred embodiments”
XXXXXXXXXXASTKGXXXXXXXXXX

SEQ ID NO: 211 - protein, 26 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (17)..(26): Any amino acid
  VARIANT (18)..(26): /replace=“ ”
  misc_feature (18)..(26): /note=“Residues given in the sequence have no preference with respect to those in the annotations for said positions”
  misc_feature (17)..(26): /note=“This region may encompass 1 to 10 residues”
  source: /note=“see specification as filed for detailed description of substitutions and preferred embodiments”
XXXXXXXXXXASTKGPXXXXXXXXXX

SEQ ID NO: 212 - protein, 27 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (18)..(27): Any amino acid
  VARIANT (19)..(27): /replace=“ ”
  misc_feature (19)..(27): /note=“Residues given in the sequence have no preference with respect to those in the annotations for said positions”
  misc_feature (18)..(27): /note=“This region may encompass 1 to 10 residues”
  source: /note=“see specification as filed for detailed description of substitutions and preferred embodiments”
XXXXXXXXXXASTKGPSXXXXXXXXXX

SEQ ID NO: 213 - protein, 28 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (19)..(28): Any amino acid
  VARIANT (20)..(28): /replace=“ ”
  misc_feature (20)..(28): /note=“Residues given in the sequence have no preference with respect to those in the annotations for said positions”
  misc_feature (19)..(28): /note=“This region may encompass 1 to 10 residues”
  source: /note=“see specification as filed for detailed description of substitutions and preferred embodiments”
XXXXXXXXXXASTKGPSVXXXXXXXXXX

SEQ ID NO: 214 - protein, 29 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (20)..(29): Any amino acid
  VARIANT (21)..(29): /replace=“ ”
  misc_feature (21)..(29): /note=“Residues given in the sequence have no preference with respect to those in the annotations for said positions”
  misc_feature (20)..(29): /note=“This region may encompass 1 to 10 residues”
  source: /note=“see specification as filed for detailed description of substitutions and preferred embodiments”
XXXXXXXXXXASTKGPSVFXXXXXXXXXX

SEQ ID NO: 215 - protein, 30 aa, Artificial Sequence; identical to SEQ ID NO: 75
  source: /note=“Description of Artificial Sequence: Synthetic polypeptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (21)..(30): Any amino acid
  VARIANT (22)..(30): /replace=“ ”
  misc_feature (22)..(30): /note=“Residues given in the sequence have no preference with respect to those in the annotations for said positions”
  misc_feature (21)..(30): /note=“This region may encompass 1 to 10 residues”
  source: /note=“see specification as filed for detailed description of substitutions and preferred embodiments”

SEQ ID NO: 216 - protein, 18 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (13)..(18): Any amino acid
  VARIANT (14)..(18): /replace=“ ”
  misc_feature (14)..(18): /note=“Residues given in the sequence have no preference with respect to those in the annotations for said positions”
  misc_feature (13)..(18): /note=“This region may encompass 1 to 6 residues”
  source: /note=“see specification as filed for detailed description of substitutions and preferred embodiments”
XXXXXXXXXXSQXXXXXX

SEQ ID NO: 217 - protein, 19 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (14)..(19): Any amino acid
  VARIANT (15)..(19): /replace=“ ”
  misc_feature (15)..(19): /note=“Residues given in the sequence have no preference with respect to those in the annotations for said positions”
  misc_feature (14)..(19): /note=“This region may encompass 1 to 6 residues”
  source: /note=“see specification as filed for detailed description of substitutions and preferred embodiments”
XXXXXXXXXXSQPXXXXXX

SEQ ID NO: 218 - protein, 20 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (15)..(20): Any amino acid
  VARIANT (16)..(20): /replace=“ ”
  misc_feature (16)..(20): /note=“Residues given in the sequence have no preference with respect to those in the annotations for said positions”
  misc_feature (15)..(20): /note=“This region may encompass 1 to 6 residues”
  source: /note=“see specification as filed for detailed description of substitutions and preferred embodiments”
XXXXXXXXXXSQPKXXXXXX

SEQ ID NO: 219 - protein, 21 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (16)..(21): Any amino acid
  VARIANT (17)..(21): /replace=“ ”
  misc_feature (17)..(21): /note=“Residues given in the sequence have no preference with respect to those in the annotations for said positions”
  misc_feature (16)..(21): /note=“This region may encompass 1 to 6 residues”
  source: /note=“see specification as filed for detailed description of substitutions and preferred embodiments”
XXXXXXXXXXSQPKAXXXXXX

SEQ ID NO: 220 - protein, 22 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (17)..(22): Any amino acid
  VARIANT (18)..(22): /replace=“ ”
  misc_feature (18)..(22): /note=“Residues given in the sequence have no preference with respect to those in the annotations for said positions”
  misc_feature (17)..(22): /note=“This region may encompass 1 to 6 residues”
  source: /note=“see specification as filed for detailed description of substitutions and preferred embodiments”
XXXXXXXXXXSQPKATXXXXXX

SEQ ID NO: 221 - protein, 23 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (18)..(23): Any amino acid
  VARIANT (19)..(23): /replace=“ ”
  misc_feature (19)..(23): /note=“Residues given in the sequence have no preference with respect to those in the annotations for said positions”
  misc_feature (18)..(23): /note=“This region may encompass 1 to 6 residues”
  source: /note=“see specification as filed for detailed description of substitutions and preferred embodiments”
XXXXXXXXXXSQPKATPXXXXXX

SEQ ID NO: 222 - protein, 24 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (19)..(24): Any amino acid
  VARIANT (20)..(24): /replace=“ ”
  misc_feature (20)..(24): /note=“Residues given in the sequence have no preference with respect to those in the annotations for said positions”
  misc_feature (19)..(24): /note=“This region may encompass 1 to 6 residues”
  source: /note=“see specification as filed for detailed description of substitutions and preferred embodiments”
XXXXXXXXXXSQPKATPSXXXXXX

SEQ ID NO: 223 - protein, 25 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (20)..(25): Any amino acid
  VARIANT (21)..(25): /replace=“ ”
  misc_feature (21)..(25): /note=“Residues given in the sequence have no preference with respect to those in the annotations for said positions”
  misc_feature (20)..(25): /note=“This region may encompass 1 to 6 residues”
  source: /note=“see specification as filed for detailed description of substitutions and preferred embodiments”
XXXXXXXXXXSQPKATPSVXXXXXX

SEQ ID NO: 224 - protein, 26 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (21)..(26): Any amino acid
  VARIANT (22)..(26): /replace=“ ”
  misc_feature (22)..(26): /note=“Residues given in the sequence have no preference with respect to those in the annotations for said positions”
  misc_feature (21)..(26): /note=“This region may encompass 1 to 6 residues”
  source: /note=“see specification as filed for detailed description of substitutions and preferred embodiments”
XXXXXXXXXXSQPKATPSVTXXXXXX

SEQ ID NO: 225 - protein, 31 aa, Artificial Sequence; identical to SEQ ID NO: 98
  source: /note=“Description of Artificial Sequence: Synthetic polypeptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (26)..(31): Any amino acid
  VARIANT (27)..(31): /replace=“ ”
  misc_feature (27)..(31): /note=“Residues given in the sequence have no preference with respect to those in the annotations for said positions”
  misc_feature (26)..(31): /note=“This region may encompass 1 to 6 residues”
  source: /note=“see specification as filed for detailed description of substitutions and preferred embodiments”

SEQ ID NO: 226 - protein, 4 aa, Artificial Sequence
  source: /note=“Description of Artificial Sequence: Synthetic peptide”
EVQL

SEQ ID NO: 227 - protein, 35 aa, Artificial Sequence
  source: /note=“Description of Artificial Sequence: Synthetic polypeptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (26)..(35): Any amino acid
  VARIANT (27)..(35): /replace=“ ”
  misc_feature (27)..(35): /note=“Residues given in the sequence have no preference with respect to those in the annotations for said positions”
  misc_feature (26)..(35): /note=“This region may encompass 1 to 10 residues”
  source: /note=“see specification as filed for detailed description of substitutions and preferred embodiments”
XXXXXXXXXXGGGGSGGGGSGGGGSXXXXXXXXXX

SEQ ID NO: 228 - protein, 35 aa, Artificial Sequence; identical to SEQ ID NO: 227
  source: /note=“Description of Artificial Sequence: Synthetic polypeptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (26)..(35): Any amino acid
  VARIANT (27)..(35): /replace=“ ”
  misc_feature (27)..(35): /note=“Residues given in the sequence have no preference with respect to those in the annotations for said positions”
  misc_feature (26)..(35): /note=“This region may encompass 1 to 10 residues”
  source: /note=“see specification as filed for detailed description of substitutions and preferred embodiments”

SEQ ID NO: 229 - protein, 22 aa, Artificial Sequence
  source: /note=“Description of Artificial Sequence: Synthetic peptide”
  MOD_RES (1)..(10): Any amino acid
  VARIANT (2)..(10): /replace=“ ”
  misc_feature (2)..(10): /note=“Residues given in the sequence have no preference with respect to those in the annotations for said positions”
  misc_feature (1)..(10): /note=“This region may encompass 1 to 10 residues”
  MOD_RES (13)..(22): Any amino acid
  VARIANT (14)..(22): /replace=“ ”
  misc_feature (14)..(22): /note=“Residues given in the sequence have no preference with respect to those in the annotations for said positions”
  misc_feature (13)..(22): /note=“This region may encompass 1 to 10 residues”
  source: /note=“see specification as filed for detailed description of substitutions and preferred embodiments”
XXXXXXXXXXGAXXXXXXXXXX

SEQ ID NO: 230 - protein, 20 aa, Mus sp.
METDTLLLWVLLLWVPGSTG

SEQ ID NO: 231 - protein, 6 aa, Artificial Sequence
  source: /note=“Description of Artificial Sequence: Synthetic 6xHis tag”
HHHHHH

SEQ ID NO: 232 - protein, 12 aa, Artificial Sequence
  source: /note=“Description of Artificial Sequence: Synthetic peptide”
GQGTLVTVSSAS

SEQ ID NO: 233 - protein, 13 aa, Artificial Sequence
  source: /note=“Description of Artificial Sequence: Synthetic peptide”
GQGTLVTVSSAST

SEQ ID NO: 234 - protein, 14 aa, Artificial Sequence
  source: /note=“Description of Artificial Sequence: Synthetic peptide”
GQGTLVTVSSASTK

SEQ ID NO: 235 - protein, 15 aa, Artificial Sequence
  source: /note=“Description of Artificial Sequence: Synthetic peptide”
GQGTLVTVSSASTKG

SEQ ID NO: 236 - protein, 16 aa, Artificial Sequence
  source: /note=“Description of Artificial Sequence: Synthetic peptide”
GQGTLVTVSSASTKGP

SEQ ID NO: 237 - protein, 17 aa, Artificial Sequence
  source: /note=“Description of Artificial Sequence: Synthetic peptide”
GQGTLVTVSSASTKGPS

SEQ ID NO: 238 - protein, 18 aa, Artificial Sequence
  source: /note=“Description of Artificial Sequence: Synthetic peptide”
GQGTLVTVSSASTKGPSV

SEQ ID NO: 239 - protein, 19 aa, Artificial Sequence
  source: /note=“Description of Artificial Sequence: Synthetic peptide”
GQGTLVTVSSASTKGPSVF

SEQ ID NO: 240 - protein, 20 aa, Artificial Sequence
  source: /note=“Description of Artificial Sequence: Synthetic peptide”
GQGTLVTVSSASTKGPSVFP

SEQ ID NO: 241 - protein, 11 aa, Artificial Sequence
  source: /note=“Description of Artificial Sequence: Synthetic peptide”
GSGTQLTVLSQ

SEQ ID NO: 242 - protein, 12 aa, Artificial Sequence
  source: /note=“Description of Artificial Sequence: Synthetic peptide”
GSGTQLTVLSQP

SEQ ID NO: 243 - protein, 13 aa, Artificial Sequence
  source: /note=“Description of Artificial Sequence: Synthetic peptide”
GSGTQLTVLSQPK

SEQ ID NO: 244 - protein, 14 aa, Artificial Sequence
  source: /note=“Description of Artificial Sequence: Synthetic peptide”
GSGTQLTVLSQPKA

SEQ ID NO: 245 - protein, 15 aa, Artificial Sequence
  source: /note=“Description of Artificial Sequence: Synthetic peptide”
GSGTQLTVLSQPKAT

SEQ ID NO: 246 - protein, 16 aa, Artificial Sequence
  source: /note=“Description of Artificial Sequence: Synthetic peptide”
GSGTQLTVLSQPKATP

SEQ ID NO: 247 - protein, 17 aa, Artificial Sequence
  source: /note=“Description of Artificial Sequence: Synthetic peptide”
GSGTQLTVLSQPKATPS

SEQ ID NO: 248 - protein, 18 aa, Artificial Sequence
  source: /note=“Description of Artificial Sequence: Synthetic peptide”
GSGTQLTVLSQPKATPSV

SEQ ID NO: 249 - protein, 19 aa, Artificial Sequence
  source: /note=“Description of Artificial Sequence: Synthetic peptide”
GSGTQLTVLSQPKATPSVT

SEQ ID NO: 250 - protein, 24 aa, Artificial Sequence
  source: /note=“Description of Artificial Sequence: Synthetic peptide”
GSGTQLTVLSQPKATPSVTGGGGS